(19) United States
(12) Patent Application Publication
(10) Pub. No.: US 2017/0119643 A1
(43) Pub. Date: May 4, 2017

(54) DETOXIFICATION OF MICROBIAL VIRULENCE FACTORS IN ORAL CAVITY

(71) Applicant: The Procter & Gamble Company, Cincinnati, OH (US)

(72) Inventors: John Christian HAUGHT, West Chester, OH (US); Sancai XIE, Liberty Township, OH (US); Cheryl Sue TANSKY, Forest Park, OH (US); Yogita Vinod MANTRI, Cincinnati, OH (US); Thomas Glenn HUGGINS, Mason, OH (US); Donald James WHITE, Fairfield Township, OH (US)

(21) Appl. No.: 15/149,044

(22) Filed: May 6, 2016

Related U.S. Application Data
(60) Provisional application No. 62/157,671, filed on May 6, 2015.
(60) Provisional application No. 62/157,659, filed on May 6, 2015.
(60) Provisional application No. 62/309,110, filed on Mar. 16, 2016.

(51) Int. Cl. A61K 8/49; A61K 8/36; A61K 8/02; A61K 8/42; A61K 8/21; A61Q 11/00; G01N 33/50; A61K 8/39

(52) U.S. Cl. CPC A61K 8/4926; G01N 33/5041; A61K 8/361; A61K 8/39; A61K 8/42; A61K 8/21; A61Q 11/00; A61K 8/0204; G01N 2333/70596; G01N 2333/195; A61K 2800/74

(57) ABSTRACT

A method for identifying constituents for detoxifying oral virulence factors and a method for determining the effectiveness of detoxifying agents.

### FIELD OF THE INVENTION

The invention relates to a method for identifying agents that detoxify oral virulence factors and a method for determining the effectiveness of detoxifying agents.

### BACKGROUND OF THE INVENTION

The toxins of bacterial cell walls (Henkel et al, EXS. (2010) 100: 1-29) have been associated with health related issues, such as septic shock, fever and malaise (V. Liebers et al., Am J Ind Med. (2006) 49(6): 474-91). Examples of Gram-negative cell wall toxins associated with health concerns are endotoxins, such as lipopolysaccharide (LPS), peptidogylcans, and fimbriae; and Gram-positive cell wall toxins associated with health concerns are lipoteichoic acid (LTA) and peptidoglycans. There are many other bacterial toxins, such as enterotoxins and exotoxins, as reported in Henkel et al, EXS. 2010; 100: 1-29. For the oral environment, the LPS and LTA seem to be the dominant drivers of a bacterial induced immune response, or at least the best characterized. The immune response mounted by the body in response to these toxins depends on the origin of the toxin and the exposure history of the individual to said toxin. The LPS is a component of Gram-negative bacteria that is different from strain to strain, as has been illustrated with the differences in virulence of E. coli (Raetz and Whitfield Annu. Rev. Biochem (2002) 71:635-700). LPS is composed of a lipid A fraction, core region, and may have an O-antigen. The Lipid A fraction's fatty acid composition has been shown to determine its virulence in response to its interaction with the Toll-like 4 (TLR4) receptor. The LTA has been linked to various inflammatory responses (Y. Yokoyama, et al., Acta Otolaryngol Suppl. (1996) 523:108-111) and associated with Toll-like receptor 2 (TLR2) activation. It is widely believed that only lysed bacteria liberate LPS that can initiate an inflammatory response (CA2323630). However, Zhang et al. showed that growing bacteria secrete LPS at a level in proportion to their growth phase (H. Zhang et al. (1998) Infection & Immunity, 66(11), 5196-5201). Therefore, even a small fraction of the plaque left on the teeth after brushing could seed the inflammatory cascade due to the release of LPS from the gram negative bacteria present in the plaque.

Methods of detecting specific microbial species have been demonstrated in the art. In US Pub. No. 2012/019735A1, methods were proposed to distinguish disease-causing bacteria via spectrophotometric methods. Though they were able to show the presence of specific microbes, their invention would not allow the user to determine the virulence level of a specific site. Further, their method requires the microbes to be cultured in the lab to obtain a sufficient quantity of LTA or LPS for detection. Thus, their invention lacks the ability to detect the non-culturable species present, and would not allow for measuring the toxicity of biological samples.

In U.S. Pat. No. 5,175,089, the use of the Limulus amebocyte lysate (LAL) endotoxin (LPS) assay was applied to the determination of the amount of endotoxin in the periodontal pocket. Though they were able to show overall amounts of endotoxin present, they lacked the ability to differentiate diseased versus healthy endotoxin and they were unable to quantify the level of virulence of the endotoxin. Further, their invention limited them to the Gram-negative endotoxin, as the LTA is not detectable via the LAL kit.

TLR signal transduction involves association of the chaperonin 10 with a Toll-like receptor in an activation cluster. In US Pub. No. 2009/0047240, the chaperonin 10 (Cpn10) antibodies were used to modulate the clustering of Cpn10 in a cell line, murine RAW264, thus changing the signal transduction of TLR. Though they showed TLR-4, 7, and 9 reporter genes in an HEK cell line, their system would not allow for a more sensitive or low level detection needed for microbial populations with weaker activating LPS, since those genes were under the control of the NFkB binding sites only (a minimal promoter). Their system lacks the sensitivity needed to differentiate biological systems with multiple microbial species and no dominant organism present. Further, their system needs strong NFkB activators to overcome the weak promoter used in their system, thus unable to pick up weaker TLR LPS agonists, such as LPS from Porphyromonas gingivalis. Additionally, their system lacked the ability to detect TLR3 agonists, which would be deleterious to the characterization of an inflammatory disease, such as gingivitis.

US Pub. No. 2007/0160544 describes a method for determining orally deleterious bacteria. Their method calls for contacting a gingival cell with bacteria or a bacterial component and measuring an inflammatory marker. According to US Pub. No. 2007/0160544, the presence of a marker indicates inflammation and the labeling of a bacterium as deleterious. Conversely, they say that the absence of a marker indicates the bacterium is not a problem. Though they cited Toll-like receptors, which were known in the art as part of the pathway to generate cytokines, their method would have only allowed for determining the presence of a cytokine. The methods outlined in '544 were predicated on the workings of Dongari-Bagtzoglou and Ebersole in J. Perio. Res. 31: 90-8 1996, where Dongari showed that human gingival fibroblast cells could be used to screen bacteria for an inflammatory response by measuring the production of either IL-1 beta, IL-6, or IL-8 cytokines. The '544 application was analogous to the work of Madiano et al., in J. Clinical Perio. 32: 57-71 2005, where Madiano reviewed the literature around bacterial virulence factors and disclosed the concept of gingival cells as methods to determine the anti-inflammatory properties of multiple bacterial components from several species of bacteria. Their system proposes the use of indicator materials (cytokines) generated from the host immune response by the activation of any Toll-like receptor. Though their proposed system may give rise to a cytokine, as was demonstrated in the literature (Shapira et al., J. Dent. Res. 76: 1381-6 1997), their proposed invention would not allow for quantification of the specified cytokine, nor would it allow for determination of the virulence potential of the bacterial components. Thus, what is needed is an invention that can quantify virulence and determine level of potency.

Since oral cells contain one or more of the receptors to which a bacterial virulence factor would activate, screening on the individual receptors requires the use of engineered cells, such as reporter cells containing the receptor gene of interest. What further complicates the use of native oral cells, such as gingival cells, is that the expression and activation of a receptor, such as a Toll-like receptor, is specific to the function of the cell. Gingival cells are less likely to respond to bacterial virulence factors, due to their constant contact with microbes in the dental plaque. Thus the need exists to have engineered cells where a direct response can be measured via a reporter system.

In addition to quantifying the virulence of microbial components and byproducts, there also exists a need for an in vitro screen of the inflammatory potential of organic and inorganic molecules, which would allow for pharmokinetic parameters to be determined.

### SUMMARY OF THE INVENTION

A method for identifying agents for detoxifying oral virulence factors is provided that comprises providing a reporter cell that expresses at least one of TLR2 or TLR4; providing an oral virulence factor; providing a potential detoxifying agent; combining oral virulent factor and potential detoxifying agent with reporter cell; determining EC50 of virulence factor.

A method to identify detoxifying agents that bind to at least one of lipopolysaccharide or lipoteichoic acid is provided that comprises providing a reporter cell that expresses at least one of TLR2 or TLR4; providing at least one of lipopolysaccharide or lipoteichoic acid; combining at least one of lipopolysaccharide or lipoteichoic acid and potential detoxifying agent with reporter cell; measuring binding of potential detoxifying agents using at least one of BODIPY method or LAL method of low EC50 lipopolysaccharide or lipoteichoic acid; and determining EC50 of at least one of lipopolysaccharide or lipoteichoic acid.

An oral care composition is provided that comprises a detoxifying agent that is at least one of myristyl-gamma-picolinium chloride; potassium linoleate; Sodium hexadecyl sulfate; fenugreek; steapyrium chloride; alpha.-glyceryl monooloeate; stearyldiethanolamine; Dimethylaminopropyllaurylamide; Stearylamidopropyldimethylamine; linoleamide; N-Lauryldiethanolamine; Steartrimonium chloride; hexaethylene glycol, monodecyl ether; Laureth-6; Laureth-9; Quaternium-17; Bromoenol lactone; Oleyl amine; cetrimonium chloride; or palmitamide monoethanolamine.

### BRIEF DESCRIPTION OF THE DRAWINGS

FIG. 1A has a graph showing secretion of LPS during bacterial growth in culture.

FIG. 1B has a graph showing the time course of bacterial growth in culture medium.

FIG. 1C has a graph showing secretion of LPS during bacterial growth in culture.

FIG. 1D has a graph showing secretion of LPS during bacterial growth in culture at 24 hours.

FIG. 1E has a graph showing the amount of LPS per bacterium during bacterial growth in culture at 24 hours.

FIG. 2A has a graph showing cell culture and virulence determination of lipopolysaccharide from different bacteria.

FIG. 2B has a graph showing bacteria activation of the TLR4 reporter gene in culture.

FIG. 2C has a graph showing the supernatant of bacterial growth activates TLR4 reporter gene in culture.

FIG. 3 has a graph showing stannous fluoride inhibited TLR4 reporter gene expression stimulated by E. coli LPS.

FIG. 4 has a graph showing stannous fluoride prevented P. gingivalis LPS from activating TLR2 reporter gene expression.

FIG. 5 has a graph showing percentage reduction of P. gingivalis LPS on activation of TLR-4 by stannous fluoride.

FIG. 6 has graphs showing EC50 determination of subgingival plaques that activate TLR4-SEAP signal transduction.

FIG. 7 has a graph showing TLR4 Evaluation of Human Subgingival Plaque (Healthy vs. Gingivitis).

FIG. 8A has a graph showing cell culture and virulence determination of LTA from different bacteria.

FIG. 8B has a graph showing cell culture and virulence determination of LPS from different bacteria.

FIG. 8C has a graph showing cell culture and virulence determination of LPS from different bacteria.

FIG. 8D has a graph showing cell culture and virulence determination of LPS from different bacteria.

FIG. 9 has graphs showing EC50 determination of subgingival plaques that activate TLR2-SEAP signal transduction.

FIG. 10 has a graph showing TLR2 Evaluation of Human Subgingival Plaque (Healthy vs. Gingivitis).

FIG. 11 has graphs showing cell culture and virulence determination of flagellins from different bacteria.

FIG. 12 has graphs showing LPS Stimulated TLR Activity in Human Monocytes THP-1. THP-1 cells produce several functional TLR receptors.

FIG. 13 has a graph showing detection of LTA of different bacteria in THP1 dual reporter cells.

FIG. 14 has graphs showing clinical measurements.

FIG. 15 has a graph showing changes in the abundance of the total amount of DNA in the marginal gingival plaques.

FIG. 16A has graphs showing decreases in bacterial abundance in the supragingival plaques during the six week of regimen treatment.

FIG. 16B has graphs showing decreases in relative abundance of bacteria in the supragingival plaques during the six week of regimen treatment.

FIG. 17 has a graph showing citrulline concentrations decreased in Buccal-brush samples over the period of six-week regimen treatment.

FIG. 18 has a graph showing protein bound ornithine in Buccal brush samples decreased during treatment.

FIG. 19 has a diagram showing enzymes in the ornithine, citrulline and arginine cycle.

FIG. 20 has graphs showing expression of ornithine, citrulline and arginine genes was changed during a 6-week treatment.

FIG. 21 has a graph showing citrulline concentrations increased in Buccal-brush samples in experimental gingivitis.

FIG. 22 has graphs showing protein bound citrulline in Buccal brush samples decreased in experimentally induced gingivitis.

FIG. 23 has graphs showing concentrations of protein bound ornithine increased in Buccal-brush samples in experimentally induced gingivitis.

FIG. 24 has graphs showing concentrations of total ornithine increased in Buccal-brush samples in experimentally induced gingivitis.

FIG. 25 has graphs showing concentrations of protein bound arginine decreased in Buccal-brush samples in experimentally induced gingivitis.

FIG. 26 has graphs showing concentrations of total arginine decreased in Buccal-brush samples in experimentally induced gingivitis.

FIG. 27 has graphs showing citrulline inhibited cytokine production stimulated by P. gingivalis LPS 1690 in THP-1 cells.

FIG. 28 has a graph showing bacterial LTA reduces fluorescent intensity of BC fluorescent dye in a dose dependent manner.

FIG. 29 has a graph showing LPS reduces the fluorescent intensity of BC fluorescent dye in a dose-dependent manner.

FIG. 30 has a graph showing the supernatant of bacterial culture reduces the fluorescent intensity of BC fluorescent dye in a dose-dependent manner.

FIG. 31 has a graph showing bacteria reduce the fluorescent intensity of BC fluorescent dye in a dose-dependent manner.

FIG. 32 has a graph showing stannous fluoride binds to E. coli LPS. Binding of stannous fluoride to LPS was examined using MALDI-TOF mass spectrometry.

FIG. 33 has graphs showing stannous fluoride prevents LPS from activation of TLR-reporter gene in THP-1 cells.

FIG. 34 has graphs showing stannous fluoride inhibits LPS from stimulating production of cytokines in human primary monocytes.

FIG. 35 has a graph showing stannous fluoride removes suppression of LPS on production of cytokines in human primary monocytes.

FIG. 36 has pictures showing E. coli LPS binding to TLR4 receptors.

FIG. 37A has graphs showing bacteria stimulated production of proinflammatory cytokines in human primary blood mononuclear cells.

FIG. 37B has graphs showing bacteria stimulated production of proinflammatory cytokines in human primary blood mononuclear cells.

FIG. 37C has graphs showing bacteria stimulated production of proinflammatory cytokines in human primary blood mononuclear cells.

FIG. 37D has graphs showing bacteria stimulated production of proinflammatory cytokines in human primary blood mononuclear cells.

FIG. 37E has graphs showing bacteria stimulated production of proinflammatory cytokines in human primary blood mononuclear cells.

FIG. 38A has graphs showing LPS stimulated production of proinflammatory cytokines in human primary blood mononuclear cells.

FIG. 38B has graphs showing LPS stimulated production of proinflammatory cytokines in human primary blood mononuclear cells.

FIG. 38C has graphs showing LPS stimulated production of proinflammatory cytokines in human primary blood mononuclear cells.

FIG. 38D has graphs showing LPS stimulated production of proinflammatory cytokines in human primary blood mononuclear cells.

FIG. 38E has graphs showing LPS stimulated production of proinflammatory cytokines in human primary blood mononuclear cells.

### DETAILED DESCRIPTION OF THE INVENTION

As disclosed herein, it was surprisingly discovered that the invention could be used to detect and quantify the virulence in subgingival and supragingival plaques, thus distinguishing healthy gingivae from an inflamed site suffering from the symptoms of gingivitis. The present invention can use non-gingival engineered cells to determine the oral cavity state of health by detecting the level of bacterial toxins, such as endotoxins. Additionally, the way a host responds to the virulence factors can be determined by utilizing the host's metabolic pathways, such as quantifying products of the urea cycle.

Virulence factors are molecules produced by a microbe, such as a bacteria, fungi, or protozoa, or is a microbial agent, such as a virus, both of which contribute to the pathogenicity of the microbe or microbial agent with regard to a host inflammatory response. Non-limiting examples of virulence factors include one or more of the following: lipopolysaccharide from gram negative bacteria; lipoteichoic acid from gram positive bacteria; nucleic acids from bacteria, such as DNA and/or RNA; proteins and/or enzymes from microbes, such as a protease, lipase, or coagulase; guanosine triphosphate hydrolase enzyme hijacking agents, such as Salmonella protein SopE, which may accelerate host cell colonization; fimbriae; flagellum; adhesions; exopolysaccharide; exotoxins, such as tetanus toxin; siderophores; cytotoxin; superantigens; and pili.

Detoxification, detoxify, and/or detox as used herein refer to the inactivation of virulence factors. Non-limiting examples of virulence factors are lipopolysaccharide or endotoxin from gram negative bacteria; or lipoteichoic acid from gram positive bacteria. The inactivation of a virulence factor is defined as the virulence factor losing its ability to stimulate a host immune response. A non-limiting example of the stimulation of the host immune response would be the activation of one or more of the Toll-like Receptors, such as TLR-4, where lipopolysaccharide may have a level of activation on this receptor. A reduction of the activation of this receptor by 30% or more would fall under the definition of detoxification. Further, detoxification may be the removal of the virulence factor from the system.

Toll-like receptors (TLR) are a class of proteins in eukaryotic cells that play a role in the immune response. The class currently has 13 proteins identified and may grow as research expands in this area. They are typically abbreviated TLR-#, where the # is 1 to 13. Some of these are membrane-spanning proteins that reside on the surface of the eukaryotic cell and allow a response to the environment around the cell. Specific responses include the detection of pathogenic bacterial components, such as lipopolysaccharide from gram negative bacteria. Additionally, some of the TLR proteins have been referred to as pattern recognition receptors or pathogen-associated molecular patterns (PAMPs).

Gingivitis is defined per the FDA monograph (12 CFR Part 356, Vol. 68, No. 103 (2003)) as “An inflammatory lesion of the gingiva that is most frequently caused by dental plaque. Gingivitis is characterized by tissue swelling and redness, loss of stippling (a normal state in which the surface of healthy gingiva is comprised of small lobes), glossy surface, and increased tissue temperature. The gingiva also may bleed upon gentle provocation, such as tooth brushing or may bleed spontaneously. Gingivitis is usually not painful.” Within the monograph, plaque is defined as being composed of multiple bacterial species. Those species exert a constant inflammatory pressure on the host tissues.

When the inflammation progresses to the state of gingivitis, there exists a need to quantify how severe it is and how effective treatments from oral hygiene products are in reducing the inflammatory response. The reduction in inflammatory response due to activation of membrane bound receptors across the gingival cells is termed detoxification and measuring the level of detoxification is needed to educate consumers on the efficacy of their oral hygiene, which is lacking in the art.

Pathogenesis of gingivitis involves both bacteria and host responses. The present invention discloses methodologies measuring the virulence factors in the dental plaques in vitro, and also measuring effects of virulence factors on gingival tissues in vivo. The methodologies allow understanding of what virulence factor types are present in the dental plaques, and how the host responds. Importantly, those measurements provide a detailed assessment on the severity of gingivitis in terms of virulence factors of the microbes in dental plaques and healthy status in the host. In addition, these methods help evaluate the effectiveness of a technology in preventing and treating gingivitis.

The methodology of the present invention includes: (1) using an endotoxin detection assay kit or BODIPY-TR cadaverine method to quantify total LPS and LTA present in a biological sample and to detect technologies that inhibit the endotoxin detection assay; (2) using Toll like receptor assays to determine potency of purified virulence and oral dental plaques, and to measure the efficacy of technologies that neutralize toxicity of virulence factors; (3) using meta-sequencing to identify and quantitate bacteria in supragingival plaques, (4) measuring ornithine and citrulline in gingival swab samples to determine the healthy status of gingival tissue, (5) determining citrulline activities in inhibiting LPS-induced production of proinflammatory cytokines, (6) measuring binding of SnF₂ to LPS, and (7) determining the effective dose 50 of bacteria and LPS on stimulating cytokine production in human peripheral blood mononuclear cells.

The above-mentioned methods and chemistry may be applied in a strip form to the outer surface of the tooth and gumline. The strip may contain color or fluorescence reagents to interact with the virulence factors present and thus allow for semi-quantitative determination of the virulence present. This would allow for rapid assessment of the level of severity of gingivitis and/or periodontal disease or to ascertain the effectiveness of oral products. This type of execution of the described methods would allow for consumers to ascertain the state of their dental health at home or allow professionals, such as dentists, to rapidly measure the state of a patient's oral health.

In practicing the present invention, a strip of material may be applied to the desired oral surface by the wearer. The side of the material facing the oral surface is at least the side wherein the composition herein is applied. This oral care composition provides a vehicle for the active as well as tackiness between the oral surfaces and the strip of material, holding the strip of material in place for extended periods of time. The period over which the strip of material is used may be, for example, from about one to about thirty minutes.

A strip could comprise one or more layers where the first layer may contain an antibacterial agent and biofilm/plaque dissolving agent, such as an essential oil. The first layer would dissolve leaving a second layer containing the detoxifying agent. The third layer would comprise a backing agent. Alternatively, the first two layers may be entirely dissolving without the need for a backing agent. In addition in certain embodiments, the strip may be a single dissolvable layer containing a detoxifying agent.

The strip of material readily conforms to the oral care surface by lightly pressing it there against. The strip of material is easily removed by the wearer by peeling it off using a finger or fingernail. In certain embodiments each successive treatment uses a fresh strip of material.

In the situation were the oral care surface is the surface of teeth, it is not necessary to prepare the teeth surface before applying the delivery system of the present invention. For example, the wearer may or may not choose to brush his teeth or rinse his mouth before applying the delivery system. The surfaces of the teeth are not required to be dried or to be excessively wet with saliva or water before the strip of material is applied.

The delivery system herein may optionally be applied with two fingers to the oral surface in need of treatment and allowed to remain in place for at least five minutes. The delivery system may be applied to cover at least a portion of a tooth and at least a portion of the adjoining soft tissue (gum) area.

The methods described above can be used to determine the cellular impact of organic and inorganic molecules, for example if there is an interaction between the molecule and the targeted receptor. Further, the methods of the present invention could be used to ascertain if a molecule would cause an irritation or inflammatory response, if the receptor for that response is known. The receptor in question could be used in a reporter system as described herein and the molecular impact of the molecule in question determined. The EC50 value of the molecule in question could be determined on the receptor associated with the biological response, thus reducing or eliminating the need to do animal testing.

Metabolic changes occur in gingival tissue during gingivitis, including the degradation and synthesis of carbohydrates, lipids, proteins and nucleic acids and their metabolic products. Those changes will alter the levels of RNA, nucleic acids, carbohydrates, lipids, fatty acids, proteins, peptides, amino acids and their metabolic products in the gingival tissues, saliva, gingival crevicular fluid and dental plaques. Such biological active molecules are very potent on stimulating inflammatory responses or promoting tissue repairs. Consequently, metabolic changes in gingivitis either facilitate resolution of gingivitis or exacerbate inflammatory or injurious damages to the periodontal tissue.

Citrulline and ornithine are two amino acids in the urea cycle. Citrulline was first isolated from watermelon in 1914 by Koga and Odake. It is a key intermediate in the urea cycle pathway. Citrulline is also produced as a byproduct of an enzymatic reaction catalyzed by nitric oxide synthases. The latter oxidize arginine into nitric oxide and citrulline. Nitric oxide is an important vasodilator and also contributes to host defence and seems to have cytostatic and cytotoxic effects against certain pathogens. In addition, some Gram-positive bacteria, such as Staphylococcus aureus, can also convert arginine to nitric oxide and citrulline. Omithine is one of the products of arginases, which hydrolyze arginine into ornithine and urea.

Both citrulline and ornithine are not coded for by any gene codons, that is, not in any newly synthesized proteins. However, both host and bacterial enzymes can modify arginine residues in the proteins into either citrulline or ornithine residues. As a result, the modified proteins either lose their original functions or gain new activities. Importantly, bacteria produce virulence factors, which possess enzymes to convert arginine in proteins to ornithine or citrulline, thus, modifying the function of the proteins to cause pathogenic conditions or help tissue heal. Metabolic changes also alter the production and expression of cytokines, chemokines, extra cellular matrix proteins and other cellular proteins. Similarly, metabolic changes shift different population of immune cells. All those changes, including small molecules related to carbohydrates, lipids, proteins and nucleic acids in the gingival tissue, will reflect the healthy status of gingival tissues, and can function as biomarkers individually. As a whole, those changes will give a detailed assessment of the potency and levels of virulence factors in the oral cavity.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. (i.e. room temperature) unless otherwise specified

As used herein, the word “include,” and its variants, are intended to be non-limiting, such that recitation of items in a list is not to the exclusion of other like items that may also be useful in the materials, compositions, devices, and methods of this invention.

As used herein, the word “or” when used as a connector of two or more elements is meant to include the elements individually and in combination; for example X or Y, means X or Y or both.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include hair coloring compositions, oral care compositions, after shave gels and creams, pre-shave preparations, shaving gels, creams, or foams, moisturizers and lotions, cough and cold compositions, leave-on skin lotions and creams, shampoos, conditioners, shower gels, bar soaps, toilet bars, antiperspirants, deodorants, depilatories, lipsticks, foundations, mascara, sunless tanners and sunscreen lotions.

By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice”, as used herein, includes tooth or subgingival-paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “teeth”, as used herein, refers to natural teeth as well as artificial teeth or dental prosthesis.

The present invention may include obtaining a sample of oral matter. The oral matter can include gum-line plaque, subgingival plaque, supragingival plaque, interstitial plaque, gingival crevicular fluid (GCF), gingival biopsy, saliva, or tongue swab. The oral matter may be obtained by any method known in the art, for example, subgingival plaque sample may be collected physically by scraping or by using paper points. The plaque may be collected off of the tooth beneath gums from the sulcus, the developed periodontal pocket, or at the gumline. For example, each paper point can be placed in the pocket between the tooth and the gingiva for 10 seconds. After 10 seconds, a paper point can be removed and placed into a pre-labeled 1.5 ml tube with 700 μl phosphate-buffered saline. The sampling procedure can be repeated with three more paper points. After all four paper points are collected, the 1.5 ml tube will be closed, vortexed for 30 seconds and placed on dry ice until the samples are stored in a −80° C. freezer. Other methods of collection could include a mechanical device to help release the plaque from the tooth surface, such as a sonic descaler. The oral matter may be obtained both before and after treatment of an oral site from which the oral matter is obtained. An oral site from which the oral matter is obtained includes host tissues and bacterial matters. Further treatment of an oral site may be more than once and may include multiple different treatments, for example a regimen, such as brushing teeth followed by mouthrinse. In addition to obtaining oral matter before and after completed treatments, oral matter may be obtained between separate treatments, for example between the brushing of teeth followed by the use of mouth rinse.

The sample of oral matter is combined with a Toll-like receptor in a reporter cell. Examples of Toll-like Receptors that can be used in the present invention include TLR 2, TLR 4, TLR 5, and TLR 9. Human TLR cDNA (TLR 1, 2, 3, 4, and 5) was first cloned in 1998 and their sequences were published (Rock F L, Hardiman G, Timans J C, Kastelein R A, Bazan J F. A family of human receptors structurally related to Drosophila Toll. Proc Natl Acad Sci USA. 1998 Jan. 20; 95:588-93). Rock et al. discovered that cytoplasmic domains of Drosophila Toll and human interleukin 1 receptors shared high sequence homology, and hypothesized that both molecules trigger related signaling pathways tied to the nuclear translocation of Rel-type transcription factors. They cloned a class of putative human receptors with a protein architecture that is similar to Drosophila Toll in both intra- and extracellular segments. They hypothesized that the five human Toll-like receptors—named TLRs 1-5—are probably the direct homologs of the fly molecule and, as such, could constitute an important and unrecognized component of innate immunity in humans. Soon after the DNA sequences for TLR1 to 5 were published, other TLR DNA sequences were also revealed. For example, TLR6 cDNA sequence was reported in 1999 (Takeuchi O1, Kawai T, Sanjo H, Copeland N G, Gilbert D J, Jenkins N A, Takeda K, Akira S. A novel member of an expanding toll-like receptor family. Gene. 1999 Apr. 29; 231(1-2):59-65). cDNA sequences of human TLR7, TLR8 and TLR9 were reported in 2000 (Chuang T H1, Ulevitch R J. Cloning and characterization of a sub-family of human toll-like receptors: hTLR7, hTLR8 and hTLR9. Eur Cytokine Netw. 2000 September; 11(3):372-8).

| SEQ ID NO | Sequence |
|---|---|
| 1 | Human Toll-Like Receptor 1 Amino Acid sequence |
| 2 | Human Toll-Like Receptor 2 Amino Acid sequence |
| 3 | Human Toll-Like Receptor 3 Amino Acid sequence |
| 4 | Human Toll-Like Receptor 4 Amino Acid sequence |
| 5 | Human Toll-Like Receptor 5 Amino Acid sequence |
| 6 | Human Toll-Like Receptor 6 Amino Acid sequence |
| 7 | Human Toll-Like Receptor 7 Amino Acid sequence |
| 8 | Human Toll-Like Receptor 8 Amino Acid sequence |
| 9 | Human Toll-Like Receptor 9 Amino Acid Sequence |

A sequence listing that sets forth the amino acid sequences for SEQ ID NO: 1 to 9 herein is being filed concurrently with the present application as an ASCII text file titled “13838M_AA_Sequence_Listing_ST25.” The ASCII text file was created on 6 Apr. 2016 and is 68 Kbytes in size. In accordance with MPEP §605.08 and 37 CFR §1.52(e), the subject matter in the ASCII text file is incorporated herein by reference.

TLR proteins can form heterodimers or homodimers. There are 10 TLR genes identified in humans. Their gene products form homodimers or heterodimers in cell membranes. For example, TLR1 can form heterodimers with TLR. Similarly, TLR6 also can assemble a heterodimer with TLR2. On other hand, TLR4 forms a homodimer. Toll-like receptors (TLRs) are a class of proteins that play a key role in the innate immune system as well as the digestive system. They are membrane-spanning, non-catalytic receptors usually expressed in cells such as macrophages, dendrite cells, and gingival epithelial cells that recognize structurally conserved molecules derived from microbes. Once these microbes have breached physical barriers such as the skin or intestinal tract mucosa or oral epithelial cells they are recognized by TLRs, which activate immune cell responses. Toll-like receptors were targeted because they are the primary sensors of virulence factors produced by the microbes in the dental plaques. TLR 1, 2, 4, 5 and 6 are expressed in the cell plasma membranes, providing easy access for the virulence factors to be sensed by the host receptors. TLR 3, 7, 8, and 9 are located on the membranes of endosomes; and as virulence factors are phagocytosed into the cells, they also gain access to TLR 3, 7, 8 and 9

A reporter cell refers to a eukaryotic cell, such as, but not limited to, HEK 293T, human monocyte (THP1), Chinese hamster ovary (CHO) cell, murine cells, or monkey kidney epithelial (Vero) cells engineered to express a predetermined number of TLR receptors, for example a single TLR receptor; which is in contrast to gingival cells that express multiple functional TLR receptors. Thus, one type of engineered reporter cells respond to only one type of virulence factors in the dental plaques. In contrast, gingival cells express several types of functional TLRs, and can't be used to identify a single type of virulence factor in dental plaques. The output from gingival cells is the sum of various virulence factors in the dental plaques. HEK 293T cells can be used as reporter cells, as they are easy to maintain and have similar gene expression profiles to oral epithelial cells, making them a closer match to the gene expression of gingival cells, such that the results will mirror in vivo results. In contrast to naturally occurring gingival cells the reporter cells of the present invention are easy to maintain in the lab, and stable in phenotypes. Further, the reporter cells make detection of virulence factors simpler, are more reproducible, increase accuracy, provide higher throughput are more specific and more quantifiable.

The receptor genes are attached to the regulatory sequence of another gene (reporter gene), such as a fluorescent or luminescent gene, which may encode green fluorescent protein, luciferase, alkaline phosphatase, and/or red fluorescent protein. An example of a reporter gene is the SEAP reporter gene. The SEAP reporter gene encodes a secreted enzyme, called embryonic alkaline phosphatase or SEAP. For instance, in certain embodiments, the SEAP reporter gene is placed under the control of an interferon-β minimal promoter fused to five NFkB and AP-1-binding sites. A recombinant Toll-like receptor binds its ligand, and initiates a chain of responses, leading to recruitment of NFkB and API transcription factors to the reporter gene promoter, which induce expression of SEAP. As compared to measuring an immune response the reporter genes of the present invention allow rapid, specific and reproducible measurements of virulence factors. Other receptors can be used in combination with Toll-like receptors, for example, nucleotide-binding oligomerization domain (NOD)1 and NOD2 recognize the peptidoglycan derivatives, meso-diaminopimelic acid (meso-DAP) and muramyl dipeptide (MDP), and trigger host innate immune responses.

The level of receptor activation can be determined by any method known in the art for the type of reporter gene used. For example if an NFkB-SEAP reporter gene is used, one could measure the production of SEAP in the culture medium. The reporter cells can be treated with virulence factors, or dental plaque matters collected before or after treatments. Expression of the reporter gene will be stimulated and SEAP secreted into the medium when stimulated by virulence factors. The level of reporter gene product SEAP can be readily measured with commercial kits, and will be proportional to the amount of particular types of virulence factors. Similarly, if an NFkB-luciferase, NFkB-beta-lactamase, or other reporter genes are used, available kits can be used to measure the reporter gene products.

The potency can then be determined based on parameters such as, EC50 and fold of stimulation. EC50 provides a measurement on the amount of virulence factors needed to mount an inflammatory response, and the fold of stimulation is indicative of the severity of inflammatory responses the virulence factors cause. The EC50 is used to determine potency; wherein “potency” as defined by the Merck Manual, refers to the concentration (EC50) of a chemistry required to produce 50% of the chemistry's maximal effect as depicted by a graded dose-response curve. EC50 equals Kd (Dissociation constant, which is a measure of 50% of the substance in question bound to the receptor) when there is a linear relationship between occupancy and response. Often, signal amplification occurs between receptor occupancy and response, which results in the EC50 for response being much less (ie, positioned to the left on the abscissa of the log dose-response curve) than Kd for receptor occupancy. Potency depends on both the affinity of a compound for its receptor, and the efficiency with which a compound-receptor interaction is coupled to response. The dose of a compound required to produce an effect is inversely related to potency. In general, low potency is important only if it results in a need to administer a compound in large doses that are impractical. Quantal dose-response curves provide information on the potency of a compound that is different from the information derived from graded dose-response curves. In a quantal dose-response relationship, the EC50 is the dose at which 50% of individuals exhibit the specified quantal effect.

In the present invention, the activities of endotoxins or lipopolysaccharides can be measured using an endotoxin detection assay, or LAL assay. The Limulus Amebocyte Lysate (LAL) test has been used to detect LPS. LAL is derived from the blood cells, or amebocytes, of the horseshoe crab, Limulus polyphemus. At present, some main endotoxin detection agents are derived from recombinant proteins. Thereinafter, endotoxin detection assay and LAL assay are used interchangeably.

In certain embodiments a determination of virulence is assigned based on the activation of one or more of the Toll-like receptors. Virulence factors include, but are not limited to the following: gram positive and gram negative cell wall components, such as lipopolysaccharide and lipoteichoic acids; bacterial DNA; flagellin; peptidoglycan; bacterial metabolites; fimbriae; outer membrane vesicles; and bacterial proteins and/or enzymes. A reduction in virulence or “detoxification”, based on reduced activation of Toll-like receptors, can be used to measure the effectiveness of various treatments. The term “detoxification” or “detoxify” or “detox” refers to the neutralization, reduction, and/or removal of microbial virulence factors as measured by a reduction in the activation of a receptor known to be responsive with a virulence factor from an engineered cell, such as a reporter cell line.

The oral care compositions of the present invention may include one or more of the following components, which can include metal salts, sweeteners, carrier materials, antimicrobial agents, bad breath reduction agents, bleaching agents (hydrogen peroxide), surfactants, flavors (menthol), anti-tartar agents, colorants, sensates, abrasive polishing materials, thickening materials, humectants, and other additives.
A. A method for identifying agents for detoxifying oral virulence factors comprising: providing a reporter cell that expresses at least one of TLR2 or TLR4; providing an oral virulence factor; providing a potential detoxifying agent; combining oral virulent factor and potential detoxifying agent with reporter cell; determining EC50 of virulence factor.
B. The method of paragraph A, wherein the virulence factor is lipopolysaccharide, preferably wherein the lipopolysaccharide is from a Gram negative bacterium.
C. The method of paragraph A, wherein the virulence factor is lipoteichoic acid, preferably wherein the lipoteichoic acid is from a Gram positive bacterium.
D. The method of according to any of paragraphs A to C, wherein the potential detoxifying agent is at least one of myristyl-gamma-picolinium chloride; potassium linoleate; Sodium hexadecyl sulfate; fenugreek; steapyrium chloride; alpha.-glyceryl monooloeate; stearyldiethanolamine; Dimethylaminopropyllaurylamide; Stearylamidopropyldimethylamine; linoleamide; N-Lauryldiethanolamine; Steartrimonium chloride; hexaethylene glycol, monodecyl ether; Laureth-6; Laureth-9; Quaternium-17; Bromoenol lactone; Oleyl amine; cetrimonium chloride; or palmitamide monoethanolamine.
E. A method to identify detoxifying agents that bind to at least one of lipopolysaccharide or lipoteichoic acid comprising:
   a) providing a reporter cell that expresses at least one of TLR2 or TLR4;
   b) providing at least one of lipopolysaccharide or lipoteichoic acid;
   c) combining at least one of lipopolysaccharide or lipoteichoic acid and potential detoxifying agent with reporter cell;
   d) measuring binding of potential detoxifying agents using at least one of BODIPY method or LAL method of low EC50 lipopolysaccharide or lipoteichoic acid; and
   e) determining EC50 of at least one of lipopolysaccharide or lipoteichoic acid.
F. An oral care composition comprising:
   a detoxifying agent that is at least one of myristyl-gamma-picolinium chloride; potassium linoleate; Sodium hexadecyl sulfate; fenugreek; steapyrium chloride; alpha.-glyceryl monooloeate; stearyldiethanolamine; Dimethylaminopropyllaurylamide; Stearylamidopropyldimethylamine; linoleamide; N-Lauryldiethanolamine; Steartrimonium chloride; hexaethylene glycol, monodecyl ether; Laureth-6; Laureth-9; Quaternium-17; Bromoenol lactone; Oleyl amine; cetrimonium chloride; or palmitamide monoethanolamine.
G. The oral care composition of paragraph F, wherein the detoxifying agent is present in an amount of from about 0.001% to about 2.0%, by weight of the oral care composition.
H. The oral care composition paragraph F or G, wherein the oral care composition is a strip.

### EXAMPLES

### Example 1—Determination of Secreted Lipopolysaccharide by Bacteria

Growth of bacteria: A 1 ml aliquot of a 24 hour culture of E. coli ATCC 8739 was used to inoculate 100 ml of Luria-Bertani (LB) broth in a 250 ml baffled flask. This culture was then incubated at 37° C. with agitation (220 rpm) and sampled at 30 minute intervals. Samples were assessed for turbidity (OD₆₀₀) in a SpectraMax platereader M3 (Molecular Devices, Sunnydale, Calif.), which is one method of monitoring the growth and physiological state of microorganisms. The sample turbidity was then recorded and the samples were centrifuged at 5000 RPM for 10 min at room temperature. The supernatant, thereinafter referred to as “supernatant of bacterial culture”, was subsequently analyzed for LPS content using the procedure as described below.

Twenty ml aliquots of MTGE broth (Anaerobe Systems, Morgan Hill, Calif.) were inoculated with P. gingivalis ATCC 33277, P. pallens ATCC 700821, or P. nigrescens ATCC 25261. These cultures were incubated overnight in a Whitely A45 Anaerobic Workstation (Don Whitley Scientific, Frederick, Md.) at 37° C. with an 85:10:5 N₂:CO₂:H₂ gas ratio. One ml aliquots of these starter cultures were then used to inoculate 99 ml of membrane-Tryptone Glucose Extract (m-TGE) broth in a 250 ml baffled flask. These cultures were then incubated under agitation (200 rpm) as previously described and sampled at regular intervals. Samples were assessed for turbidity (OD₆₀₀) in a Tecan Infinite m200 Pro (Tecan Trading AG, Switzerland) and then centrifuged at 16,100×g for 10 min at room temperature. Supernatants were decanted and passed through a 0.22 μM filter prior to analysis for LPS content.

In the experiment, only OD600 was measured. For the sake of consistency in following experiments, we converted OD600 readings into bacterial numbers, even though the relationship between OD600 readings and bacterial numbers is varied for each bacterium. The number of bacteria was estimated based on spectrophotometer readings at OD₆₀₀ (OD₆₀₀ of 1.0=8×10 cells/ml).

The Limulus Amebocyte Lysate Assay (LAL) is an assay to determine the total amount of lipopolysaccharide (LPS) in the sample tested (Pierce LAL Chromogenic Endotoxin Quantitation Kit, ThermoFischer Scientific, Waltham, Mass.). The assay was performed following manufacturer's instruction. Ninety-six-well microplates were first equilibrated in a heating block for 10 min at 37° C. Fifty μl each of standard or sample was dispensed into the microplate wells and incubated with the plate covered for 5 min at 37° C. Then 50 μl LAL was added to each well.

Plates were shaken gently and incubated for 10 min at 37° C. 100 μl of chromogenic substrate was added and incubated for 6 min at 37° C. Finally, 50 μl Stop Reagent was added and the absorbance was measured at 405-410 nm on Spectramax M3 platereader (Molecular Device, Sunnyvale, Calif.).

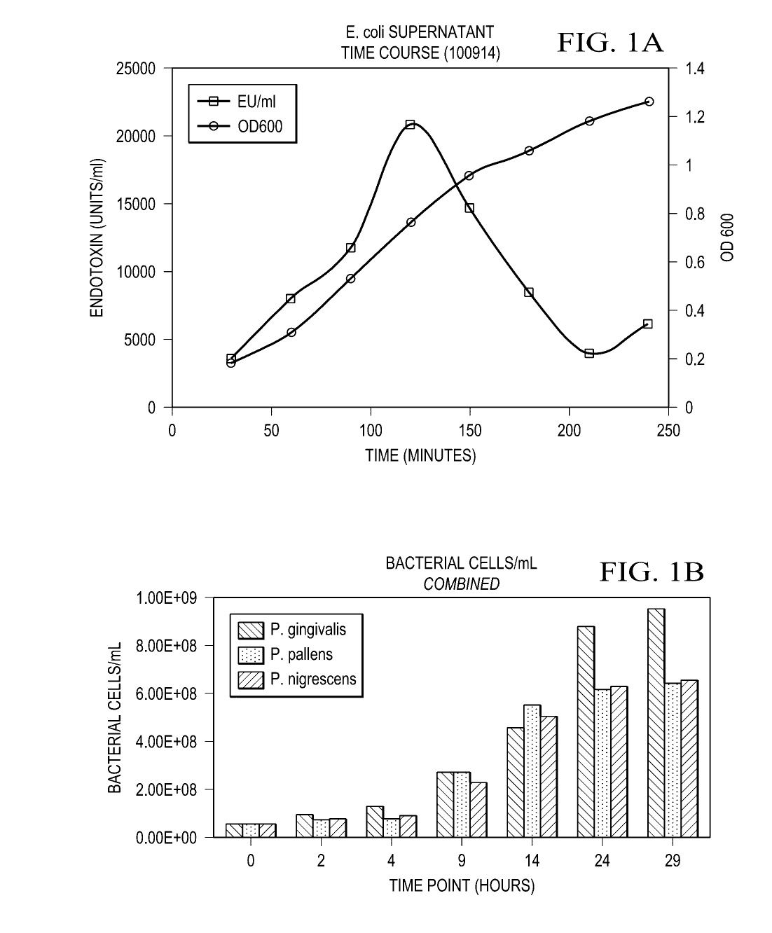

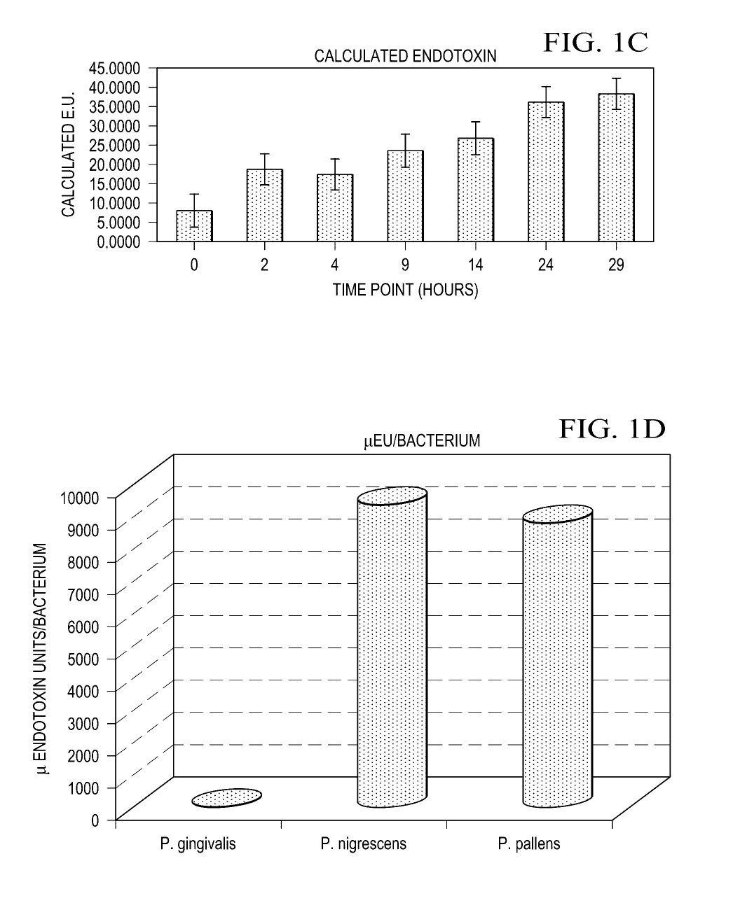

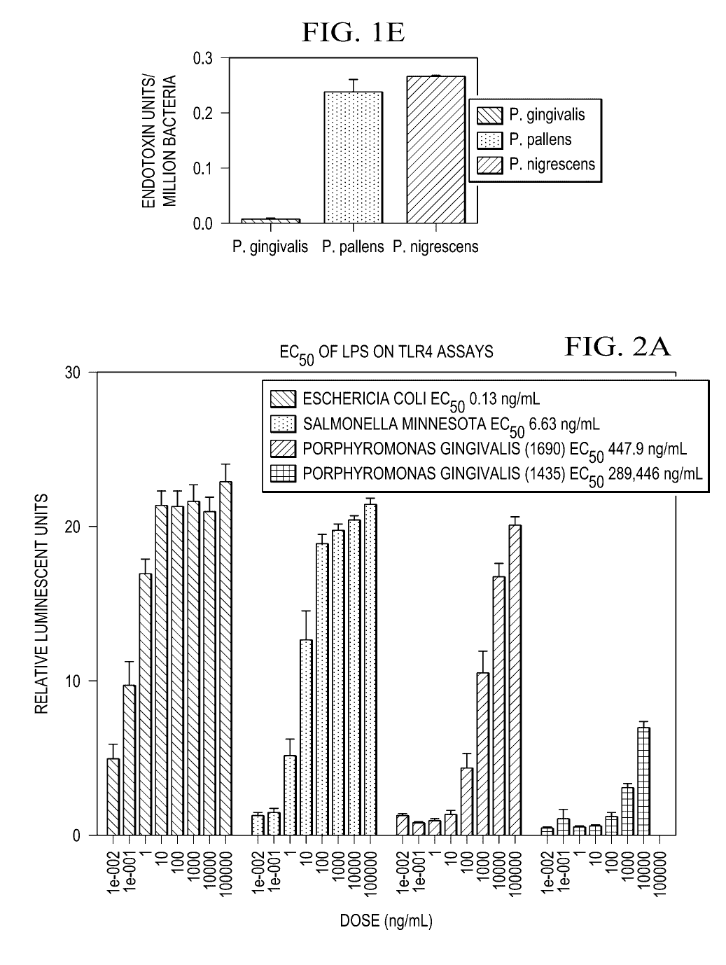

FIGS. 1A, 1C, and 1D show the ability of microbes to shed LPS as part of their normal growth cycle. This data shows the need to deliver chemistry to the subgingival plaque to effectively mitigate the LPS, since tooth brushing generally does not remove the subgingival plaque.

The LPS, as measured by the LAL kit reported in endotoxin unit per ml (EU/ml), was shed by the bacteria (E. coli K12) as depicted in FIG. 1A. The growth media began to be depleted of complex sugars around 120 minutes, as reflected in the bacterial growth curve in FIG. 1B, where the LPS shedding started to decline. This data gave a reason to believe that a mature biofilm/plaque could supply a constant level of LPS to the host cells, if food sources were present. The LPS would then have the ability to induce an inflammatory response from the host cells.

Importantly, LPS is secreted into the supernatant of bacterial culture (FIG. 1D). LPS also exists in bacterial walls (FIG. 1E). Again, this data further enforce the need to deliver chemistry to the subgingival plaque to effectively mitigate the LPS, since tooth brushing generally does not remove the subgingival plaque.

### Example 2—LAL Assay to Quantify Virulence Factors in Dental Plaques

Seven panelists, with at least three bleeding sites, took part in the testing. A licensed dental hygienist collected subgingival plaque samples. Samples were taken at the tooth/gum interface (buccal surfaces only) using care to avoid contact with the oral soft tissues. Six subgingival plaque sites were sampled from each panelist (3 healthy and 3 unhealthy sites). Unhealthy teeth had bleeding sites with pockets greater than 3 mm and healthy sites had no bleeding with pocket depth less than 2 mm. Prior to sampling, panelists were instructed to abstain for 12 hours from oral hygiene and refrain from eating, chewing gum, drinking (except small sips of water). Next, panelists had their marginal plaque collected with a curette at the sampling sites. Then, from the same site, subgingival plaque samples were collected with 3 consecutive paper points. The sampling sites were isolated with cotton rolls and gently air-dried. Paper points (PROFLOW incorporated, Amityville, N.Y.) were gently placed for 10 seconds into the pocket until a minimum of resistance was felt. After 10 seconds, paper points were removed and placed into pre-labeled 1.5 ml tubes. The same sampling procedure was repeated with 2 more paper points (paper points go into separate tubes). The first, second and third sample paper points from a healthy site of all panelists were pooled separately into three tubes, labeled as paper point 1, 2 and 3, respectively. Similarly the unhealthy site samples were also pooled.

TABLE 1 showed that unhealthy dental plaques contained more endotoxins than the healthy dental plaques. One ml PBS was added to each pooled sample in the 1.5 ml tube. Bacteria were lysed in a MolBio Fast Prep bead beater (MP Biomedicals, Santa Ana, Calif.). Samples were centrifuged for 10 min at 10,000 RPM at 4° C., supernatants were collected and analyzed with LAL assay kits following manufacturer's instruction as described in EXAMPLE 1.

TABLE 1
Protein concentrations and endotoxin levels
in the pooled dental plaque samples.

| | Endotoxin |
|---|---|
| Dental plaque | (endotoxin unit) |
| Healthy paperpoint 1 sub plaque | 1284 |
| Healthy paperpoint 2 sub plaque | 476 |
| Healthy paperpoint 3 sub plaque | 361 |
| Healthy Marginal Plaque | 23180 |
| Unhealthy paperpoint 1 sub plaque | 3371 |
| Unhealthy paperpoint 2 sub plaque | 1732 |
| Unhealthy paperpoint 3 sub plaque | 1644 |
| Unhealthy Marginal Plaque | 80277 |

It was expected that the marginal plaques in unhealthy sites had more endotoxins than those in the healthy sites (TABLE) within the same subjects. Three samples were taken from subgingival pockets with three paper points sequentially, named paper point 1, 2 and 3. Again, the subgingival plaques taken by the paper point 1 had more endotoxins in the unhealthy sites than in the healthy sites (TABLE 1). The same is true for the samples taken by paper point 2 and 3. Importantly, dental plaques in the unhealthy subgingival pockets possessed more endotoxins than plaques from healthy pockets. This may explain why unhealthy gingiva are prone to bleeding upon probing.

### Example 3—Technology Development Using the LAL Assay Kit

The LAL assay, as described in EXAMPLE 1, was modified for development of technology which inhibits LPS from activating a proenzyme in the LAL assay. The Thermo Scientific Pierce LAL Chromogenic Endotoxin Quantitation Kit is a quantitative endpoint assay for the detection of LPS, which catalyzes the activation of a proenzyme in the modified Limulus Amebocyte Lysate (LAL). The activated proenzyme then splits p-Nitroaniline (pNA) from the colorless substrate, Ac-Ile-Glu-Ala-Arg-pNA. The product pNA is photometrically measured at 405-410 nm. If SnF₂ binds to LPS, the latter can't react with the proenzyme in the LAL kit. Consequently, the proenzyme is not activated, and the colorless substrate Ac-Ile-Glu-Ala-Arg-pNA will not split and no color product is produced. P. gingivalis LPS 1690 (1 ng/ml), or E. coli LPS (1 ng/ml), and stannous fluoride and other materials (50 and 500 μM), as listed in TABLE 2, were dissolved in endotoxin-free water. Then 50 μl LAL was added to each well. Plates were shaken gently and incubated for 10 min at 37° C. 100 μl of chromogenic substrate was added and incubated for 6 min at 37° C. Finally, 50 μl Stop Reagent was added and the absorbance was measured at 405-410 nm on Spectramax M3 plate reader (Molecular Device, Sunnyvale, Calif.).

As shown in TABLE 2, SnF₂ and some other compounds inhibited LPS activities in LAL assays

TABLE 2
Inhibition of LPS activities on LAL Assays

| | Inhibition of LAL activity % | | | |
|---|---|---|---|---|
| | P. gingivalis LPS | | | |
| | 1690 1 ng/ml | | E. coli LPS 1 ng/ml | |
| Samples | 500 uM | 50 uM | 500 uM | 50 uM |
| Tin (II) fluoride | 60 | 49 | 92 | 87 |
| stannous chloride | 48 | 21 | 89 | 65 |
| Cetylpyridinium chloride | 103 | 77 | 103 | 46 |
| monohydrate | | | | |
| Chlorhexidine | 102 | 38 | 97 | 57 |
| zinc citrate, dihydrate | 104 | 57 | 104 | 82 |
| zinc lactate | 58 | 0 | 66 | 0 |
| potassium oxalate | 8 | 0 | 1 | 6 |
| Triclosan (irgasan) | 0 | 0 | 10 | 0 |
| 1-Hydroxypyridine-2-thone | 0 | 0 | 2 | 6 |
| zinc salt | | | | |
| sodium fluoride | 0 | 0 | 4 | 5 |
| Carboxymethyl cellulose | 0 | 0 | 2 | 0 |
| sodium | | | | |

### Example 4—Binding of BODIPY-TR-Cadaverine to LPS and Displacement of LPS Binding by Stannous Fluoride

In addition to LAL quantification of LPS, the BODIPY method can be utilized to assess the level of LPS. Detoxifying technologies are able to target and neutralize bacterial virulence factors, such as LPS and LTA. To develop such LPS and LTA sequestration technologies, a high throughput screening was employed to identify molecules that disrupt the activation of a Toll-like receptor by LPS and LTA, and other virulence factors. The high throughput screen utilizes the fluorescent dye BODIPY-TR-cadaverine 5-(((4-(4,4-difluoro-5-(2-thienyl)-4-bora-3a,4a-diaza-s-indacene-3-yl)phenoxy)acetyl)amino)pentylamine hydrochloride (BC fluorescent dye), purchased from Life Technologies (Carlsbad, Calif.), as described previously by Wood, Miller and David (2004) (Comb Chem High Throughput Screen 2004 May; 7(3):239-49. Anti-endotoxin agents. 1. Development of a fluorescent probe displacement method optimized for the rapid identification of lipopolysaccharide-binding agents.). The experiment was carried out at 22° C. 5 E. coli LPS (15 μg/ml) was mixed in a well of a 96-well solid black flat bottom plate (Corning Incorporated, Corning, N.Y.) with SNF₂ and cetylpyridinium chloride in 30 μl of 50 mM Tris buffer at pH 7.4 for 10 min, and then 20 μl of 60 μM BC fluorescent dye was added right before fluorescence measurement at 22° C., or room temperature. Fluorescence measurements were performed in a SpectraMax M3 automated 96 well plate reader (Molecular Device, Sunnyvale, Calif.). The excitation wavelength was 580 nM and the fluorescence emission was measured at 620 nM.

As shown in TABLE 3, leading anti-gingivitis technologies, such as stannous fluoride, displaced LPS from BODIPY TR cadaverine.

TABLE 3
Displacement of LPS from binding to BODIPY TR cadaverine

| | Displacement % | |
|---|---|---|
| Concentrations mM | Stannous fluoride | Cetylpyridinium chloride |
| 0.16 | 2.45 | 9.98 |
| 0.31 | 2.79 | 31.11 |
| 0.63 | 12.69 | 68.05 |
| 1.25 | 49.44 | 91.12 |

### Example 5—Cell Culture and Virulence Determination of Lipopolysaccharide from Different Bacteria

Reporter gene cell lines, human HEK 293T cells, were purchased from Invivogen of San Diego, Calif. The HEK 293T cells were stably transfected with at least two exogenous genes, a TLR4 structural gene, and a SEAP reporter gene, which is under the control of NFkB transcriptional factors. The cell line is named here as TLR4-SEAP. The reporter gene encodes a secreted enzyme, called embryonic alkaline phosphatase or SEAP. The SEAP reporter is placed under the control of an IL-12 p40 minimal promoter fused to five NF-κB and AP-1-binding sites. Furthermore, the TLR4-SEAP cell line also contains a CD14 co-receptor gene, which is needed to transfer LPS to TLR4 receptors. The recombinant TLR binds its ligand, or distinct pathogen-associated molecule, initiates a chain of responses, leading to recruitment of NFkB and API transcription factors to the reporter gene promoter, which induce expression of SEAP.

Cell culture and treatment: 500,000 gene reporter cells were grown and maintained in 15 ml growth medium, comprised of DMEM medium supplemented with 10% fetal calf serum in T75 flasks for three days at 37° C., 5% CO₂, and 95% humidity. For treatment, wells of a 96-well plate were seeded with 10,000 cells/well in 100 μL of growth medium. The cells were incubated for 72 hours at 37° C., 5% CO₂, and 95% humidity until day 4. On day 4, medium was changed to assay medium (90 μl), which is the DMEM medium without fetal calf serum. LPS, bacteria and the culture medium of bacterial growth, as described in EXAMPLE 1, were first resolved or mixed with the assay medium. 10 μl of the bacteria, LPS and culture medium of bacterial growth were added to the TLR4-SEAP cells. Samples were taken 24 hours later, following addition of LPS, bacteria, and culture medium. Expression of the reporter gene (SEAP) was quantified with a commercially available kit (SEAP Reporter Gene Assay of Cayman Chemical Co., Ann Arbor, Mich.).

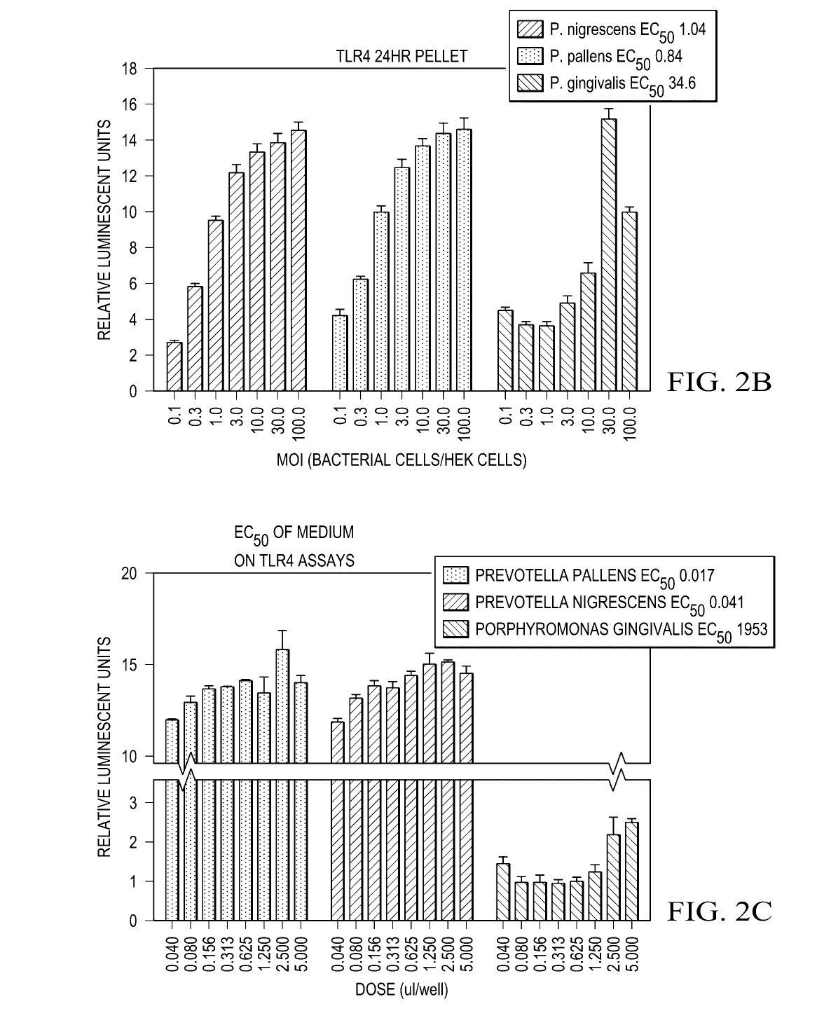

EC50 was calculated using GraphPad Prism software (GraphPad Software, La Jolla, Calif.). Samples with lower EC50 are more potent in activating the TLR4 reporter gene than those with higher EC50. As shown in FIG. 2A, LPS from E. coli has lower EC50 than P. gingivalis, thus, was far more potent than P. gingivalis (Pg). Salmonella Minnesota LPS is not as potent as that of E. coli, but is far more potent than those of P. gingivalis LPS 1690 and 1435. Each species of bacteria produces multiple forms of LPS. Each form of LPS from the same species of bacteria has different potency in stimulating TLR4-downstream signaling pathways. For example, Pg 1690 LPS is more potent than Pg1435/50. LPS is a component in bacterial cell walls. Likely, E. coli cell wall is more virulent in inducing production of proinflammatory cytokines in host cells than P. gingivalis when they are in direct contact with host blood cells. P. gingivalis had far higher EC50 than P. pallens and P. nigrescens as shown in FIG. 2B in stimulating TLR4 reporter gene expression, suggesting that P. pallens and P. nigrescens are more likely to promote production of proinflammatory cytokines in host cells than P. gingivalis.

Bacteria release LPS into the supernatant of bacterial culture. As shown in FIG. 2C, the supernatant of P. pallens has an EC50 that is similar to that of P. nigrescens, but far lower than that of P. gingivalis, in stimulating expression of TLR4 reporter gene. Again, those results imply that the products of P. pallens and P. nigrescens are more likely to promote production of proinflammatory cytokines in host cells than those of P. gingivalis.

### Example 6—Development of Technology for Inhibition of LPS Effects on Stimulating Expression of NFkB-Regulated Reporter Genes

Stannous fluoride is a leading anti-gingivitis technology in P&G toothpaste products. Tests were conducted to understand whether stannous fluoride could reduce LPS's ability to trigger proinflammatory responses in host cells. TLR4-SEAP reporter cells were prepared using the same conditions as described in EXAMPLE 5 in the presence or absence of LPS. Production of SEAP was quantified also as described in EXAMPLE 5.

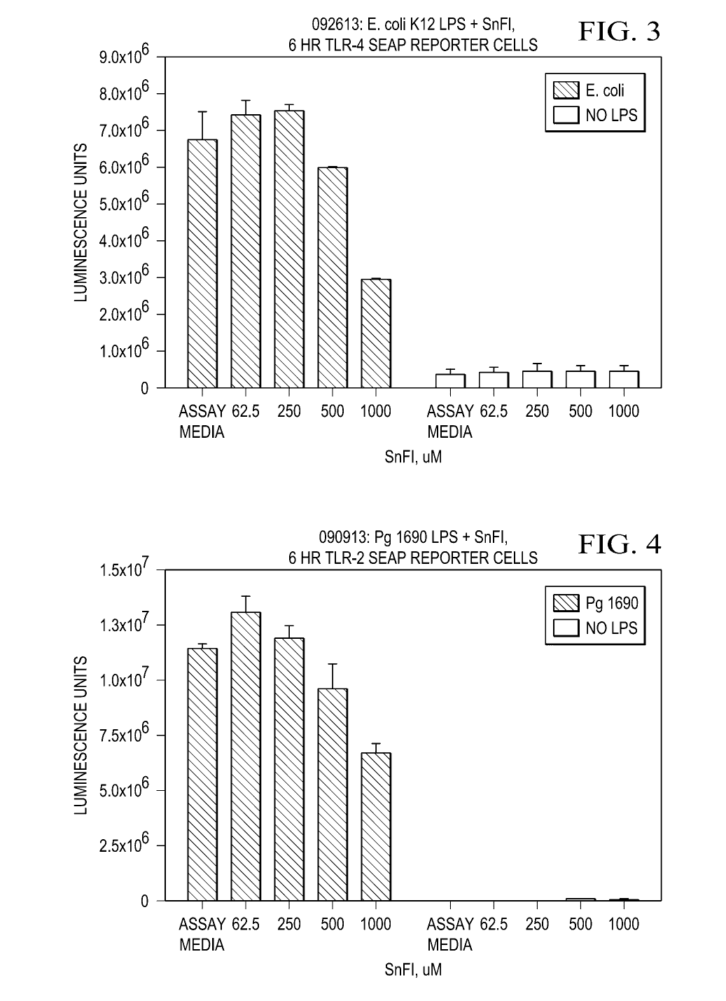

FIG. 3 shows the effect of stannous at various concentrations from 62.5 uM to 1,000 uM on 100 ng/ml E. coli LPS, as reported by activation of TLR-4. At stannous concentrations of 500 uM or higher, the level of E. coli induction of TLR-4 was decreased.

FIG. 4 shows the effects of stannous at various concentrations from 62.5 uM to 1,000 uM on P. gingivalis LPS, as reported by activation of TLR-2. At stannous concentrations of 1000 uM, the level of P. gingivalis induction of TLR-2 was decreased.

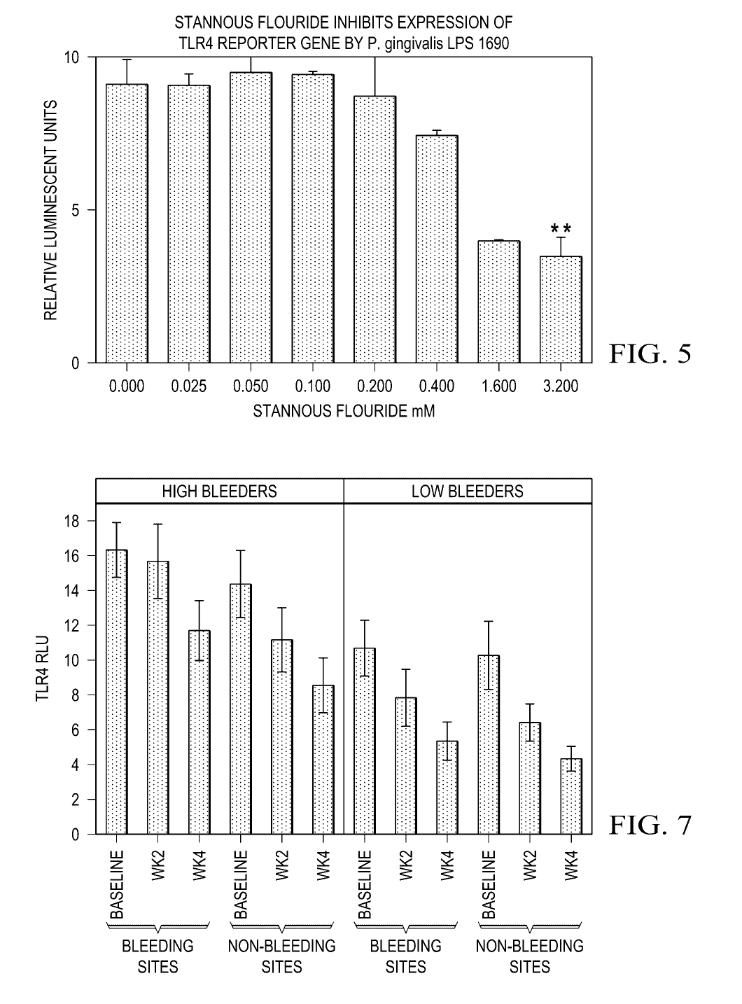

The data in FIG. 5 shows reduction of LPS activity by the stannous ion, from a stannous fluoride salt. The data showed that stannous fluoride, at 1.6 mM and 3.2 mM, reduce about 50% of P. gingivalis LPS (500 ng/ml) activation on the TLR4 reporter system (One asterisk means P<0.05, two asterisks mean P<0.01).

### Example 7—Cell Culture and Virulence Determination of EC50 in Activation of TLR4-SEAP Signal Transduction by Clinical Samples

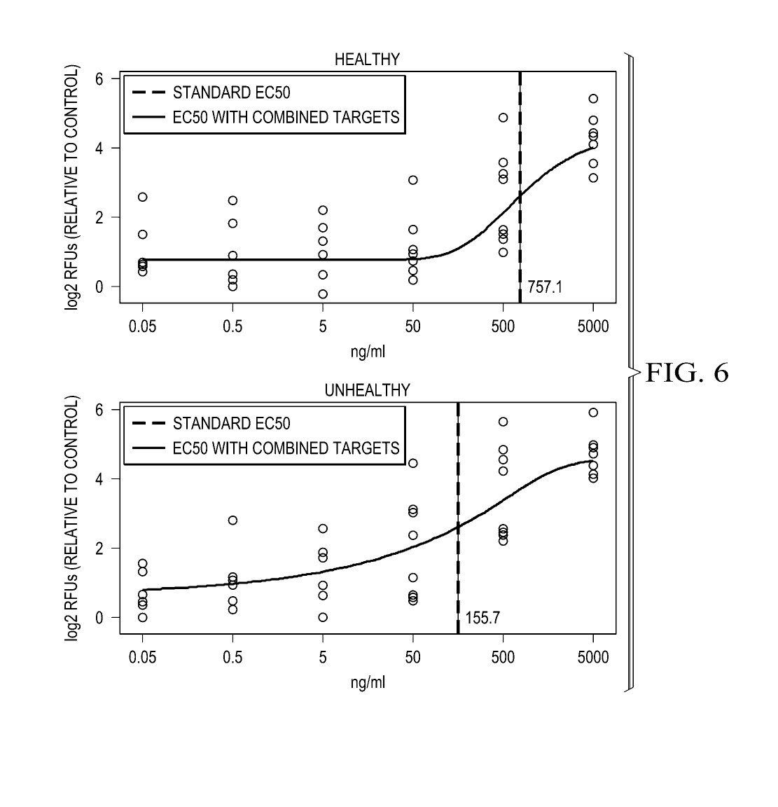

The method described in EXAMPLE 5 is effective at determining the potency of LPS from different bacteria. The same method was used to determine the EC50 of clinical samples, as described in EXAMPLE 2. As shown in FIG. 6, dental plaques from unhealthy sites had a smaller EC50 than those from healthy sites, suggesting the dental plaques from unhealthy sites contain more virulence factors.

The same method described in EXAMPLE 5 was used to examine the clinical samples in another study. A clinical study was conducted to evaluate sample collection methods and measurement procedures. It was a controlled, examiner-blind study. Forty panelists met the inclusion criteria, wherein in order to be included in the study, each panelist must:
Provide written informed consent to participate in the study;
Be 18 years of age or older;
Agree not to participate in any other oral/dental product studies during the course of this study;
Agree to delay any elective dentistry (including dental prophylaxis) until the study has been completed;
Agree to refrain from any form of non-specified oral hygiene during the treatment periods, including but not limited to the use of products such as floss or whitening products;
Agree to return for all scheduled visits and follow study procedures;
Must have at least 16 natural teeth;
Be in good general health, as determined by the Investigator/designee based on a review of the health history/update for participation in the study.

For Unhealthy Group (high bleeder group):
Have at least 20 bleeding sites (sites with a score of 1 or 2 on the GBI index); Have minimum 3 sampling sites with bleeding and pocket depth >3 mm but not deeper than 4 mm;
Have minimum 3 sampling sites without bleeding and with pocket depth <2 mm For Healthy Group (low bleeder group):
Have maximum 3 bleeding sites (sites with a score of 1 or 2 on the GBI index);
No pockets deeper than 2 mm. Twenty (20) panelists were qualified as healthy—with up to 3 bleeding sites and with all pockets less than or equal to 2 mm deep and twenty (20) panelists were qualified as unhealthy—with greater than 20 bleeding sites with at least 3 pockets greater than or equal to 3 mm but not deeper than 4 mm with bleeding, and at least 3 pockets less than or equal to 2 mm deep with no bleeding for sampling. All panelists had up to 6 sites identified as “sampling sites.” The “sampling sites” had supragingival and subgingival plaque collected at Baseline, Week 2 and Week 4. Subgingival plaque samples were taken from a gingival sulcus from the pre-identified sites. Prior to sample collection, the site had supragingival plaque removed with a curette. The site was dried and subgingival plaque samples were collected with another dental curette (e.g., Gracey 13/14, 15/16, 11/12, ⅞, ½.) Each Gracey curette is designed to adapt to a specific area or tooth surface. For example, Gracey 13/14 is designed to adapt to the distal surfaces of posterior teeth. Samples from each site were placed in a pre-labeled 2.0 ml sterile tube containing 300 μl of DPBS buffer with about 50 of sterile 1 mm glass beads. Samples were stored at 4° C. The subgingival samples were stored at −80° C. until analyzed. The samples were thawed at room temperature and dispersed in a TissueLyser II (Qiagen, Valencia, Calif., USA) at 30 shakes per second for 3 min. Protein concentrations of the dispersed subgingival samples were measured using a Pierce microBCA Protein kit (ThermoFisher Scientific, Grand Island, N.Y., USA) following the manufacturer's instruction.

Oral lavage samples were collected at wake up (one per panelist) by rinsing with 4 ml of water for 30 seconds and then expectorating the contents of the mouth into a centrifuge tube. These samples were frozen at home until they were brought into the site in a cold pack. Each panelist collected up to 15 samples throughout the study. Saliva samples were frozen at −70° C. from submission.

All panelists were given investigational products: Crest® Pro-Health Clinical Gum Protection Toothpaste (0.454% stannous fluoride) and Oral-B® Indicator Soft Manual Toothbrush. Panelists continued their regular oral hygiene routine, and did not use any new products starting from the baseline to the end of four week treatment study. During the four week treatment period, panelists brushed their teeth twice daily, morning and evening, in their customary manner using the assigned dentifrice and soft manual toothbrush.

The subgingival plaques from the above clinical study were applied to the TLR4 reporter cells in a procedure as described in EXAMPLE 5. FIG. 7 shows the results of a four-week study of 40 panelists going from baseline out over four weeks of treatment with Crest ProHealth Clinical toothpaste. The subgingival plaque samples in bleeding sites on the high bleeders group stimulated high expression of TLR4 reporter gene. More virulence in a sample elicits higher RLU (relative luminescent units) readings in the TLR4 reporter gene assay. As shown in FIG. 7, the baseline samples of the high bleeders group had higher RLU than those of the low bleeders on both the bleeding and non-bleeding sites. After treatment with Crest® Pro-Health Clinical Gum Protection Toothpaste (0.454% stannous fluoride) and Oral-B® Indicator Soft Manual Toothbrush for four weeks, the virulence was reduced at week 4 in both high and lower bleeders groups at both bleeding and non-bleeding sites.

### Example 8—Cell Culture and Virulence Determination of Lipoteichoic Acids from Different Bacteria

The reporter gene cell lines, human HEK 293T cells, were purchased from Invivogen of San Diego, Calif. The HEK 293T cells were stably transfected with at least two exogenous genes, a TLR2 structural gene, and SEAP reporter gene which is under the control of NFkB transcriptional factors. The cell line is named here as TLR2-SEAP. The reporter gene encodes a secreted enzyme, called embryonic alkaline phosphatase or SEAP. The SEAP reporter is placed under the control of the interferon-β minimal promoter fused to five NFkB and AP-1-binding sites. Furthermore, a CD14 co-receptor gene was transfected into the reporter gene cells expressing TLR2, as CD14 has been identified as a co-receptor for TLR2 ligands to enhance the TLR response. The CD14 co-receptor is needed to transfer LTA to TLR2 receptors. The recombinant TLR2 binds its ligand, or distinct pathogen-associated molecule, initiates a chain of responses, leading to recruitment of NFkB and API transcription factors to the reporter gene promoter, which induce expression of SEAP.

Cell culture and treatment: 500,000 gene reporter cells were grown and maintained in 15 ml growth medium, comprising DMEM medium supplemented with 10% fetal calf serum in T75 flasks for three days at 37° C., 5% CO₂, and 95% humidity. For treatment with LTA, wells of a 96-well plate were seeded with 10,000 cells/well in 100 μL of growth medium. The cells were incubated for 72 hours at 37° C., 5% CO₂, and 95% humidity until day 4. On day 4, medium (100 μL) was changed to DMEM medium without fetal calf serum. LTA, LPS and bacterial cells, as described in EXAMPLE 7, were added. Samples were taken 24 hours later, following addition of samples. Expression of the reporter gene (SEAP) was quantified with a commercially available kit (SEAP Reporter Gene Assay of Cayman Chemical Co., Ann Arbor, Mich.).

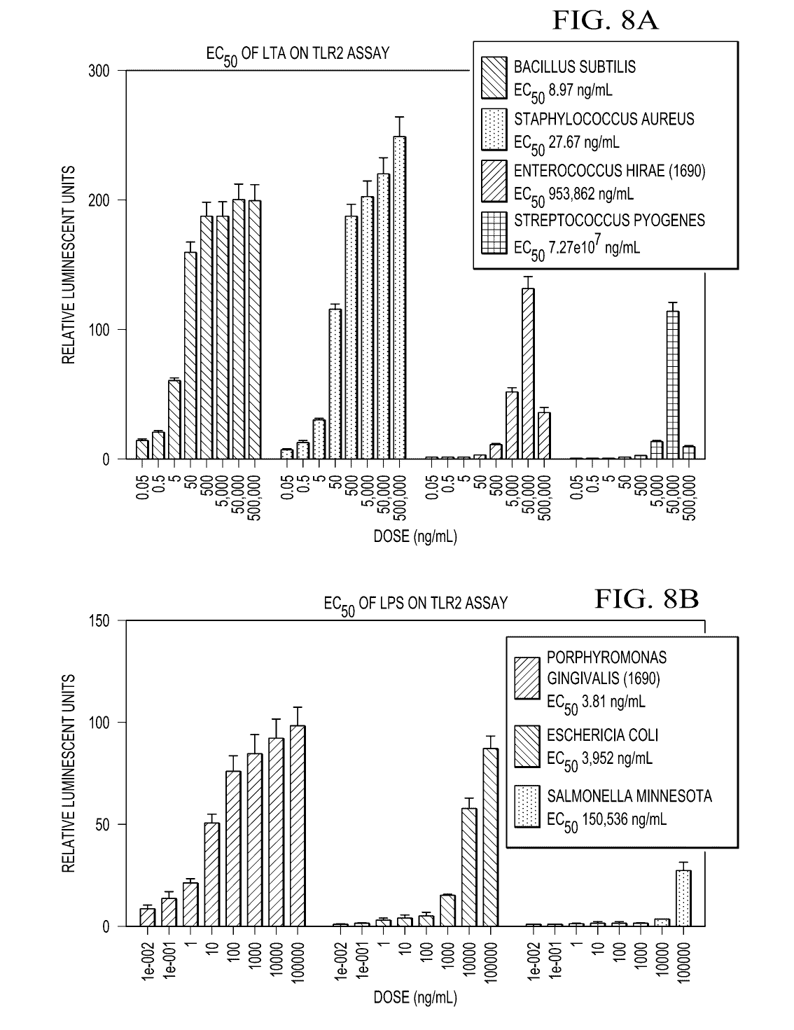

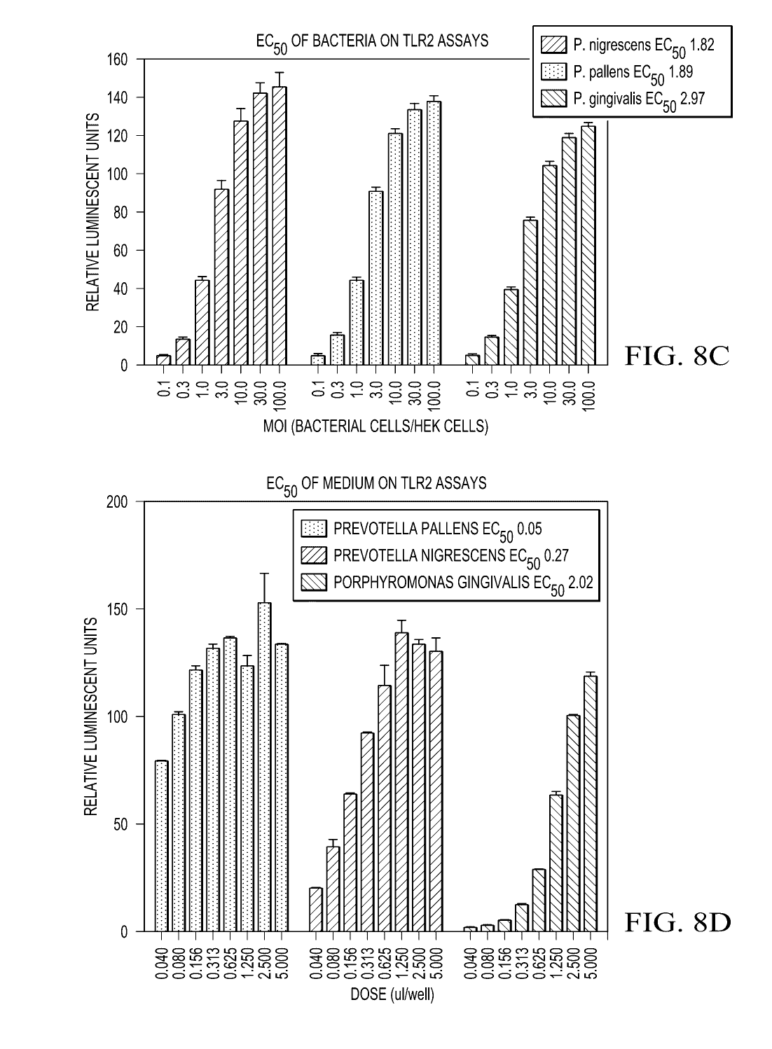

As shown in FIGS. 8A, 8B, 8C and 8D, LTA, LPS, bacteria and the supernatant of bacterial culture could bind to TLR2 and activate TLR2 downstream signaling pathways in a dose-dependent manner. As shown in FIG. 8A, B. subtilis (BS) LTA is more potent than that of Enterococcus hirae. As shown in FIG. 8B, P. gingivalis LPS also activated expression of the TLR2 reporter gene. For example, Pg1690, as shown in FIG. 8B, activated TLR2-SEAP signal transduction, and stimulated SEAP production. But as shown in FIG. 8B, E. coli LPS did not activate the TLR2-SEAP reporter cells. It should also be noted that P. pallens, P. nigrescens and P. gingivalis have similar EC50 in stimulating expression of TLR2 reporter gene (FIG. 8C). However, the released TLR2 ligands from the three different bacteria have very different EC50 on activation of TLR2 reporter gene (FIG. 8D).

### Example 9—Cell Culture and Virulence Determination of EC50 in Activation of TLR2-SEAP Signal Transduction by Clinical Samples

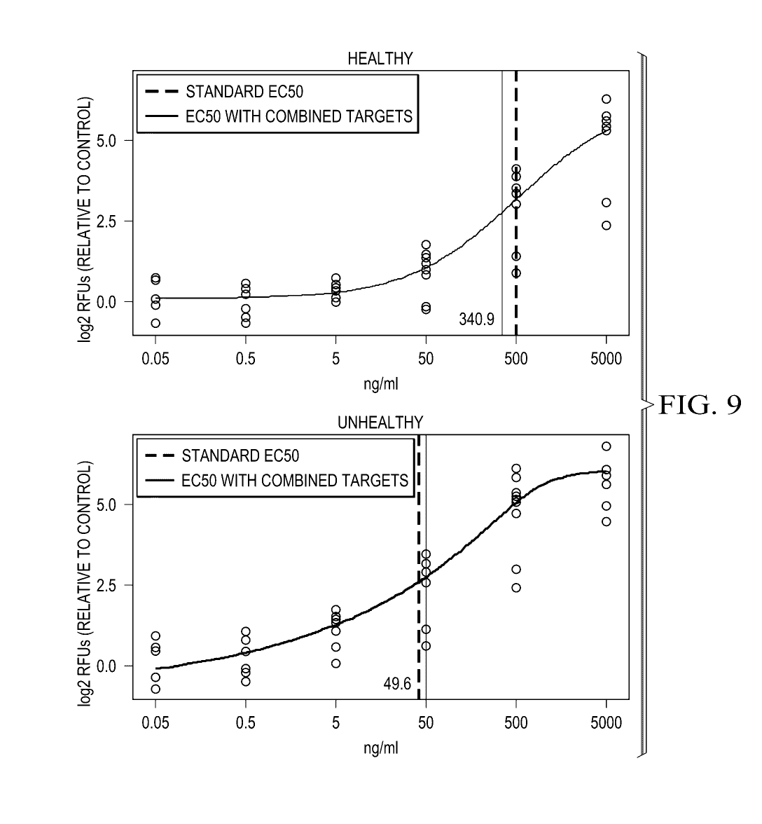

The method described in EXAMPLE 8 is effective in determining the EC50 of LTA and other TLR2 ligands from different bacteria. The same method was used to determine the EC50 of clinical samples, as described in EXAMPLE 2. As shown in FIG. 9, dental plaques from unhealthy (bleeding) sites had smaller EC50 than those from healthy (non-bleeding) sites, suggesting the dental plaques from unhealthy sites contain more virulence factors.

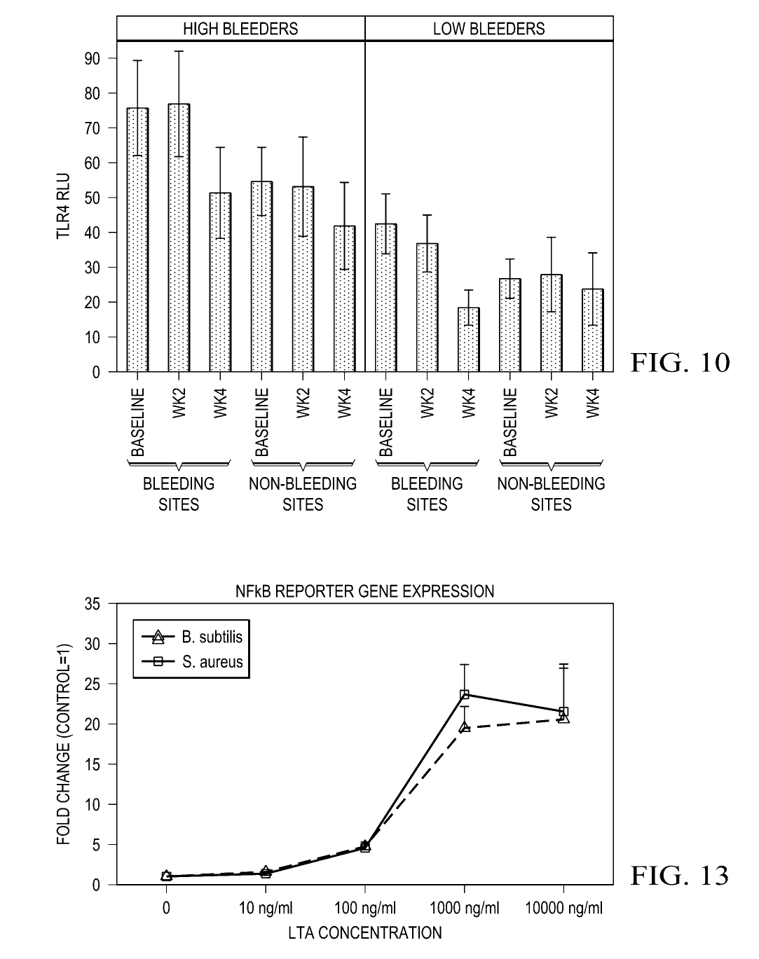

Clinical samples as described for FIG. 7 of EXAMPLE 7 were examined using the TLR2-SEAP reporter gene assay. The results are shown in FIG. 10. The subgingival samples in unhealthy (bleeding) sites from the unhealthy group (high bleeders) had more virulence factors than other sites. The baseline samples of the high bleeders group had higher RLU than those of the low bleeders on both the bleeding and non-bleeding sites. After treatment with Crest® Pro-Health Clinical Gum Protection Toothpaste (0.454% stannous fluoride) and Oral-B® Indicator Soft Manual Toothbrush for four weeks, the virulence was reduced at week 4 in both high and low bleeders groups at both bleeding sites.

### Example 10—Cell Culture and Virulence Determination of Flagellins from Different Bacteria

The reporter gene cell lines, human HEK 293T cells, were purchased from Invivogen of San Diego, Calif. The HEK 293T cells were stably transfected with two exogenous genes, a TLR5 structural gene, and SEAP reporter gene which is under the control of NFkB transcriptional factors. The cell line was named as TLR5-NFkB-SEAP. The reporter gene encodes a secreted enzyme, called embryonic alkaline phosphatase or SEAP. The SEAP reporter is placed under the control of the interferon-β minimal promoter fused to five NFkB and AP-1-binding sites. The recombinant TLR5 binds to its ligand, or distinct pathogen-associated molecule, and initiates a chain of responses leading to recruitment of NFkB and API transcription factors to the reporter gene promoter, which induce expression of SEAP.

Cell culture and treatment: 500,000 gene reporter cells were grown and maintained in DMEM medium supplemented with 10% fetal calf serum in T75 flasks for three days at 37° C., 5% CO₂, and 95% humidity. For treatment with flagellin, wells of a 96-well plate were seeded with 10,000 cells/well in 100 μL of growth medium. The cells were incubated for 72 hours at 37° C., 5% CO₂, 95% humidity until day 4 after cells were seeded onto wells of a 96-well plate. On day 4, medium (100 μL) was changed to DMEM medium without fetal calf serum. S. subtilis and S. aureus flagellin were added at a range of concentration from 0.97 ng to 1 μg/ml. Samples were taken at 6 and 24 hours later after adding flagellin. Expression of reporter gene (SEAP) was quantified with a commercially available kit (SEAP Reporter Gene Assay of Cayman Chemical Co., Ann Arbor, Mich.).

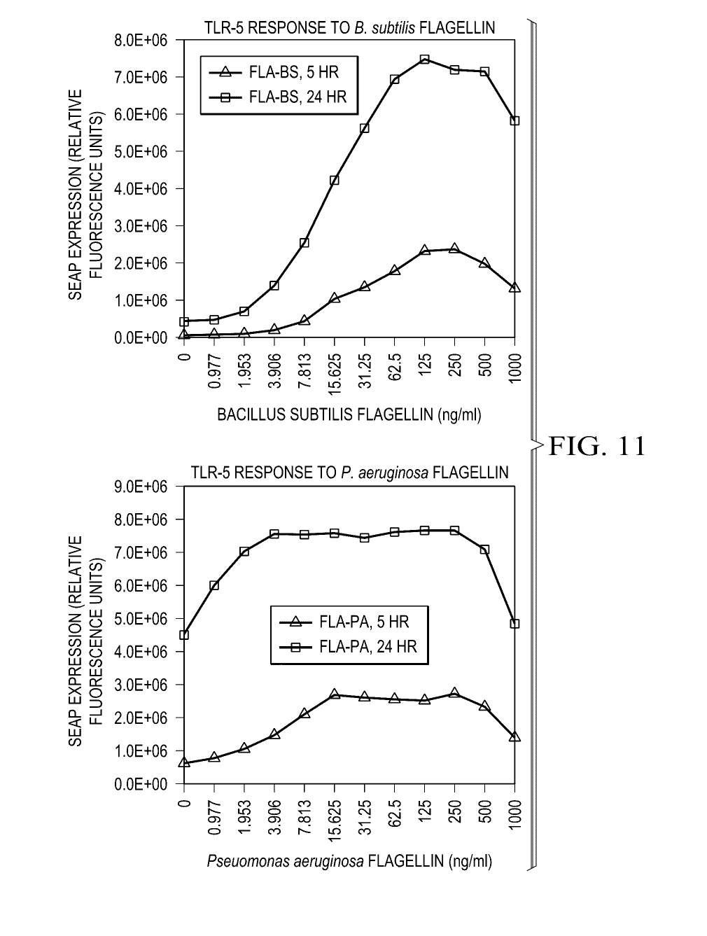

FIG. 11 shows both flagellins (B. subtilis and P. aeruginosa) could activate TLR5 downstream signaling pathways. Flagellin from B. subtilis stimulated TLR5-SEAP signaling transduction in a dose-dependent manner. The EC50 of flagellin on TLR5 was 0.02 ng/ml at 5 hours and 0.014 ng/ml at 23 hours. The EC50 of flagellin from Pseudomonas aeruginosa on TLR5 was 0.0049 at 5 hours and 0.0019 at 23 hours. Flagellin from P. aeruginosa was more potent on stimulating TLR5 signaling pathways.

### Example 11—Cell Culture and Virulence Determination of LPS from Different Bacteria in THP1 Dual Reporter Cells

Bacterial cell wall and membrane components are recognized by TLR2. TLR2 recognizes the microbial motifs PGN (peptidoglycan)/lipoproteins/dectin and LPS. TLR1 and TLR6 form heterodimers with TLR2 and bind to triacylated lipoproteins and diacylated lipoproteins, respectively. THP1 NFkB-SEAP and IRF-Lucia™ Reporter Monocytes were purchased from Invivogen, San Diego, Calif. THP1-Dual cells were derived from the human THP-1 monocyte cell line by stable integration of two inducible reporter constructs. THP1-Dual cells feature the Lucia gene under the control of an ISG54 (interferon-stimulated gene) minimal promoter in conjunction with five interferon-stimulated response elements. THP1-Dual cells also express a SEAP reporter gene driven by an IFN-b minimal promoter fused to five copies of the NF-kB consensus transcriptional response element and three copies of the c-Rel binding site. As a result, THP1-Dual cells allow the simultaneous study of the NFkB pathway, by monitoring the activity of SEAP, and the interferon regulatory factor (IRF) pathway, by assessing the activity of Lucia (IRF-Luc). Both reporter proteins are readily measurable in the cell culture supernatant. This THP-1 cell line possesses functional TLR1, TLR2, TLR4, TLR5, TLR6 and TLR8, purchased from Invivogen. TLR4 senses LPS from Gram-negative bacteria while TLR5 recognizes bacterial flagellin from both Gram-positive and Gram-negative bacteria, TLR8 detects long single-stranded RNA.

Culture and treatment: The THP1-dual cells were cultured in 15 ml growth medium (RPMI 1640 with 10% heat-inactivated fetal bovine serum) in a T75 flask at 37° C. and 5% CO₂. Cells were passed every 3 to 4 days by inoculating 300,000-500,000 cells/ml into a fresh T75 flask with 15 ml of fresh growth medium. To determine the effect of bacterial components on reporter gene expression, wells in 96-well plates were seeded at 100,000 cells in 90 μl of growth medium. 10 μl of bacterial wall and membrane components, or heat-killed whole bacteria, were added to each well. After incubation for 18 hours at 37° C. and 5% CO₂, secreted luciferase and SEAP were quantified with commercially available assay kits (QUANTI-Luc of Invivogen, San Diego, Calif. for luciferase; SEAP Reporter Gene Assay of Cayman Chemical Co., Ann Arbor, Mich. for SEAP).

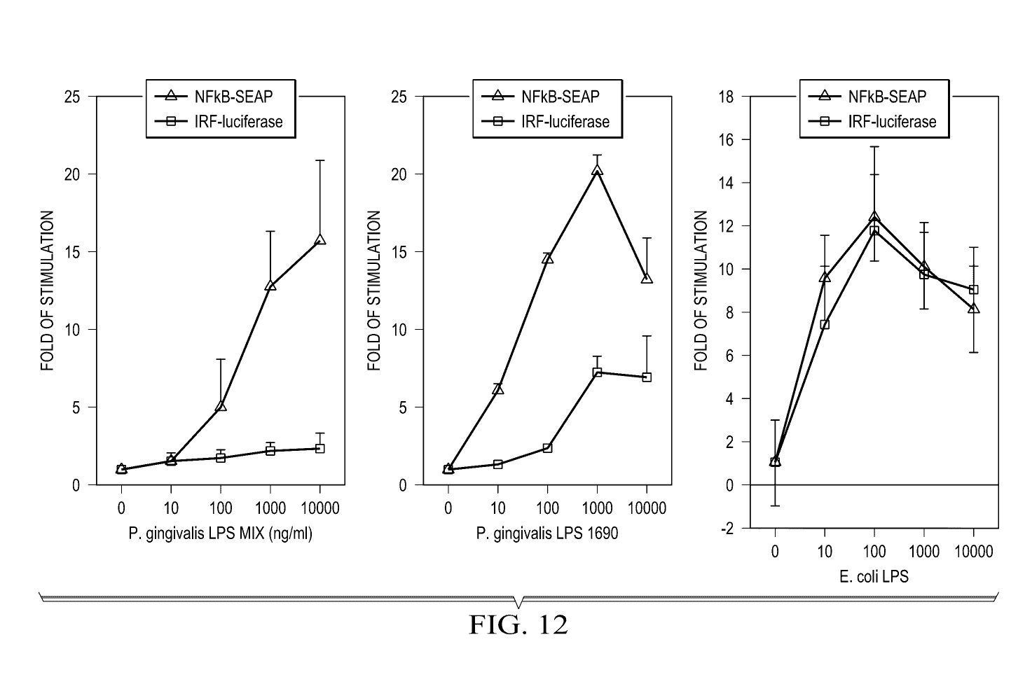

DHP1-dual reporter cells were treated with three different preparations of LPS as shown in FIG. 12. All three LPS (ng/ml) activated production of NFkB-SEAP reporter genes in a dose-dependent manner. In addition, Pg 1690 LPS and E. coli LPS also stimulated expression of the IRF-luciferase reporter gene. TLR4 ligands, upon binding to TLR4 receptors, activate at least two signaling pathways. One is a common pathway NFkB-SEAP, which can be activated by all TLR ligands upon binding to their specific receptors. For example, TLR2 ligand-LTA, can bind to TLR2 receptors and activate the NFkB-SEAP signaling pathway. Similarly, TLR4 ligand, LPS, upon binding to TLR4 receptors, also is able to activate the NFkB-SEAP signaling transduction. As shown in FIG. 12, E. coli LPS is a more potent ligand than P. gingivalis 1690 LPS on activation of both NFkB-SEAP and IRF-luciferase signaling transduction. THP-1 cells produce several functional TLR receptors. And all TLR receptors can activate the NFkB pathway, thus promoting expression of the NFkB-SEAP reporter gene. The reading of NFkB-SEAP is the collective actions of all TLR receptors, such as TLR2, TLR1, TLR6 and TLR4. All LPS from different bacteria stimulated NFkB-SEAP reporter gene. IRF-luciferase reporter gene, on the other hand, is driven by a limited number of TLR receptors, primarily TLR3, TLR4, TLR7, TLR8 and TLR9. Both P. gingivalis LPS 1690 and E. coli LPS stimulated expression of IRF-luciferase in a dose-dependent fashion.

### Example 12—Cell Culture and Virulence Determination of LTA from Different Bacteria and Biofilms in THP1 Dual Reporter Cells

THP1 dual reporter cells also express TLR2, TLR1 and TLR6 receptors. Bacterial cell wall and some membrane components are recognized by TLR2, TLR1 and TLR6. TLR2 recognizes the microbial motifs PGN (peptidoglycan)/lipoproteins/dectin and LPS. To determine whether LTA from different bacteria have different effects on stimulating NFkB-SEAP reporter gene expression in the THP1 dual reporter cells, the cells were prepared and treated in the same procedures as described in EXAMPLE 11. As shown in FIG. 13, LTA from both B. subtilis and S. aureus had similar potency in promoting reporter gene expression in the THP1 dual reporter cells.

### Example 13—Distinguishing Bacterial Components from Different Bacteria in Human Primary Gingival Epithelial Cells

Primary human gingival epithelial cells were purchased from Zen-bio (Research Triangle Park, N.C.), and maintained in 15 ml of growth medium (CellnTec medium supplemented with CellnTec Growth Supplement, purchased from CellnTec Advanced Cell Systems AG, Bern, Switzerland) in T75 flasks at 37° C. under a 5% CO₂ atmosphere. As the experiment was done at 24 h, 48 h and 72 h time point and with assay media, CellnTec medium alone or with supplements, six 96 well plates were seeded with 7,500 cells/well in 100 μl of CELLnTEC growth medium at 37° C. under a 5% CO₂ atmosphere. The growth medium was changed to assay medium right before adding LPS or bacterial DNA. For example, if the assay medium was CellnTec medium without supplements, 100 μl of CellnTec medium was added without supplements in each well. If the assay medium was the growth medium, 100 μl of growth medium was added to each well. The P. gingivalis LPS and bacterial DNA were added to the cells. At 24 h, 48 h and 72 h, medium was collected for analysis. Cytokines were measured using Elisa kits from Meso Scale Discovery (Rockville, Md.), as per the manufacturer's instructions.

As shown in TABLE 4, human primary gingival epithelial cells were treated with P. gingivalis DNA at 0, 0.3, 1 and 2 μg/ml. The cultures were harvested at 24 and 48 hours after treatment and six proinflammatory cytokines (interferon-γ, IL-1β, IL-2, IL-10, IL-12p70 and TNF-α) were analyzed using ELISA kits from Meso Scale Discovery. As shown in TABLE 4, expression of interferon-γ, IL-1β, IL-2, IL-10, IL-12p70 and TNF-α was low, or almost undetectable (each value is the mean of three replicate in one experiment). They were not viable biomarkers in distinguishing bacterial DNA. The results in TABLE 4 illustrate the lack of inflammatory response from human gingival epithelial cells, thus demonstrating the need to use an engineered cell.

TABLE 4
Screening of Human Gingival Epithelial Cells for Inflammatory
Markers treated with P. gingivalis DNA
Production of cytokines (pg/ml) in primary human gingival
epithelial cells treated with P. gingivalis genomic DNA

| | P. gingivalis | | | | P. gingivalis | | |
|---|---|---|---|---|---|---|---|
| | genomic | | | | genomic | | |
| | DNA | 24 hours | 48 hours | | DNA | 24 hours | 48 hours |
| Interferon | 0 ug | 0 | 0.25 | IL-1 | 0 ug | 0 | 0.5 |
| gamma | 0.3 ug | 0 | 0.33 | beta | 0.3 ug | 0 | 0.33 |
| | 1 ug | 0 | 0 | | 1 ug | 0 | 0 |
| | 2 ug | 0.33 | 0.67 | | 2 ug | 0 | 0 |
| IL-10 | PG DNA | 0.5 | 1 | IL- | PG DNA | 0 | 0.25 |
| | 0 ug | | | 12p70 | 0 ug | | |
| | PG DNA | 0.67 | 0.33 | | PG DNA | 0.67 | 0 |
| | 0.3 ug | | | | 0.3 ug | | |
| | PG DNA | 0 | 0 | | PG DNA | 0 | 0.67 |
| | 1 ug | | | | 1 ug | | |
| | PG DNA | 0.33 | 0.33 | | PG DNA | 0 | 0.33 |
| | 2 ug | | | | 2 ug | | |
| IL-2 | PG DNA | 0.5 | 1 | TNF- | PG DNA | 1 | 1.5 |
| | 0 ug | | | alpha | 0 ug | | |
| | PG DNA | 0 | 0.67 | | PG DNA | 1.67 | 1.33 |
| | 0.3 ug | | | | 0.3 ug | | |
| | PG DNA | 0.33 | 1 | | PG DNA | 0.67 | 1 |
| | 1 ug | | | | 1 ug | | |
| | PG DNA | 0.33 | 0.67 | | PG DNA | 1.33 | 1.33 |
| | 2 ug | | | | 2 ug | | |

Human primary gingival epithelial cells were treated with P. gingivalis LPS at 0, 0.3, 1 and 2 μg/ml in the procedures described above. As shown in TABLE 5, again, expression of interferon-γ, IL-1β, IL-2, IL-10, IL-12p70 and TNF-α was low, or almost undetectable. They were not viable biomarkers in distinguishing bacterial LPS in primary human gingival epithelial cells. The results in TABLE 5 further illustrate how human gingival cells were not sufficiently sensitive to bacterial virulence factors for an assay, and thus the need to utilize a cell line capable of demonstrating a dose dependent response to virulence factors.

TABLE 5
Screening of Human Gingival Epithelial Cells for Inflammatory
Markers treated with P. gingivalis LPS
Production of cytokines (pg/ml) in primary human gingival
epithelial cells treated with P. gingivalis LPS

| | | 24 h | 48 h | | | 24 h | 48 h |
|---|---|---|---|---|---|---|---|
| Interferon | PG lPS 0 ug | 0.00 | 0.25 | IL-1 | PG lPS 0 ug | 0.00 | 0.50 |
| gamma | PG LPS 0.3 ug | 0.00 | 0.00 | beta | PG LPS 0.3 ug | 0.00 | 0.67 |
| | PG LPS 1 ug | 0.33 | 0.33 | | PG LPS 1 ug | 0.00 | 0.00 |
| | PG LPS 2 ug | 0.00 | 0.00 | | PG LPS 2 ug | 0.00 | 1.00 |
| IL-10 | PG lPS 0 ug | 0.50 | 1.00 | IL- | PG lPS 0 ug | 0.00 | 0.25 |
| | PG LPS 0.3 ug | 0.67 | 0.67 | 12p70 | PG LPS 0.3 ug | 0.00 | 0.00 |
| | PG LPS 1 ug | 0.00 | 0.67 | | PG LPS 1 ug | 0.00 | 0.33 |
| | PG LPS 2 ug | 0.33 | 0.67 | | PG LPS 2 ug | 0.67 | 0.00 |
| IL-2 | PG lPS 0 ug | 0.50 | 1.00 | TNF- | PG lPS 0 ug | 1.00 | 1.50 |
| | PG LPS 0.3 ug | 0.33 | 0.67 | alpha | PG LPS 0.3 ug | 1.33 | 0.67 |
| | PG LPS 1 ug | 0.33 | 1.00 | | PG LPS 1 ug | 2.00 | 1.33 |
| | PG LPS 2 ug | 1.33 | 1.00 | | PG LPS 2 ug | 0.67 | 1.33 |

### Example 14—Gingivitis-Enriched Bacteria Reduced in Abundance in Six Weeks of Gingivitis Treatment

A randomized, two-group clinical study was conducted with 69 panelists (35 in the negative control group and 34 in the test regimen group). Panelists were 39 years old on average, ranging from 20 to 69, and 46% of the panelists were female. Treatment groups were well balanced, since there were no statistically significant (p≧0.395) differences for demographic characteristics (age, ethnicity, gender) or starting measurements for Gingival Bleeding Index (GBI); mean=29.957 with at least 20 bleeding sites, and Modified Gingival Index (MGI); mean=2.086. All sixty-nine panelists attended each visit and completed the treatment process. The following treatment groups were compared over a 6-week period:

Test regimen: Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse. Control regimen: Crest® Cavity Protection (0.243% sodium fluoride) dentifrice and Oral-B® Indicator Soft Manual toothbrush.

Dental plaques were collected from the same panelists in the test regimen in the clinical study as described in EXAMPLE 2. A supragingival sample was taken from each panelist with a sterile curette at the tooth/gum interface, using care to avoid contact with the oral soft tissue. Plaques were sampled from all available natural teeth (upper arch only) until no plaque was visible. Following sampling, the plaque samples were placed into a pre-labeled (panelist ID, sample initials, visit, and date) Eppendorf tube with 1 ml of PBS/Glycerol buffer and about 50 of sterile 1 mm glass beads, stored on ice until all samples were collected. The samples were then transferred to a −70° C. freezer for storage until further processing. Genomic DNA was isolated from supragingival plaque samples using QIAamp® genomic DNA kits (Qiagen, Germany) following manufacturer's instruction. Metasequencing was carried out at BGI Americas Corporation (Cambridge, Mass.). All data were analyzed at Global Biotech of Procter & Gamble Company in Mason, Ohio.

Clinical measurements: Bleeding sites (GBI) were decreased in the test regimen significantly on week 1, 3 and 6 in comparison to the control regimen (FIG. 14). Similarly, Inflammation (MGI) grades also decreased in the test regimen (FIG. 14).

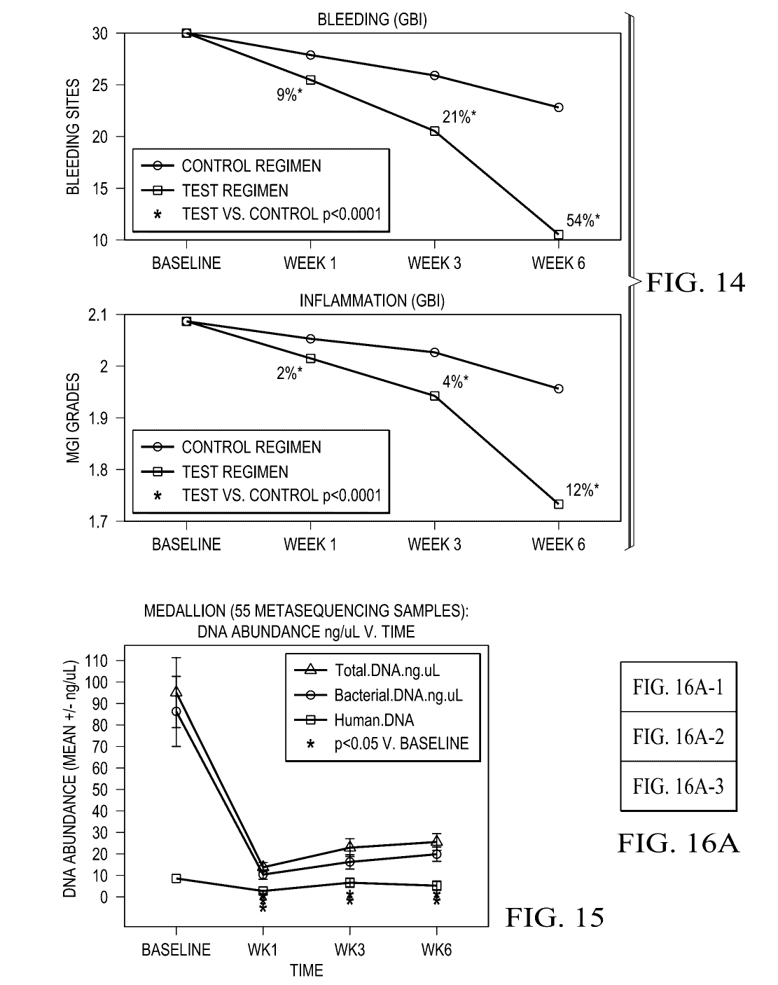

The DNA samples were isolated from margin gingival plaques which contain both human host and bacterial cells. After meta-sequencing, bacterial DNA and human DNA sequences were identified and quantitated. As shown in FIG. 15, human DNA contribution to the plaque samples was about 5-10 ng/μl from baseline to week 6 of the treatment. However, the microbial DNA was reduced dramatically from baseline to weeks 1, 3 and 6 of the treatment.

The relative abundance of bacterial genera underwent significant changes during treatment (TABLE 6A). Capnoytophaga and Campylobactor were increased during the treatment. Prevotella is a genus of Gram-negative bacteria, existing in microbiota of oral cavities, vagina and respiratory tract. This genus of bacteria can cause infections including aspiration pneumonia, lung abscess, pulmonary empyema, and chronic otitis media and sinusitis. Prevotella bacteria were abundant at baseline. Its number was reduced in weeks 1, 3 and 6.

TABLE 6A
Changes in relative abundance of bacterial genera in the
supragingival plaques from baseline to week 6 of regimen treatment.

| | Relative Abundance % | | | |
|---|---|---|---|---|
| Genera | Baseline | Week 1 | Week 3 | Week 6 |
| Streptococcus | 26.01 | 18.13 | 6.94 | 27.59 |
| Prevotella | 16.02 | 8.87 | 7.06 | 8.76 |
| Rothia | 10.04 | 12.84 | 14.75 | 15.57 |
| Veillonella | 9.93 | 5.85 | 7.64 | 7.43 |
| Actinomyces | 9.45 | 17.83 | 22.13 | 8.10 |
| Porphyromonas | 4.75 | 1.69 | 1.13 | 2.50 |
| Haemophilus | 3.97 | 4.49 | 7.24 | 7.03 |
| Gemella | 3.48 | 3.67 | 1.61 | 3.00 |
| Neisseria | 1.99 | 1.81 | 5.30 | 2.56 |
| Granulicatella | 1.87 | 2.34 | 2.23 | 2.22 |
| Atopobium | 1.26 | 1.47 | 1.18 | 0.62 |
| Megasphaera | 1.11 | 0.36 | 0.02 | 0.30 |
| Eubacterium | 1.09 | 1.14 | 0.93 | 0.99 |
| Peptostreptococcus | 1.00 | 0.42 | 0.23 | 0.55 |
| Fretibacterium | 0.88 | 1.46 | 1.20 | 0.62 |
| Solobacterium | 0.70 | 0.34 | 0.28 | 0.37 |
| Capnocytophaga | 0.55 | 2.13 | 3.27 | 1.50 |
| Alloprevotella | 0.51 | 0.45 | 1.03 | 0.38 |
| Stomatobaculum | 0.48 | 0.32 | 0.06 | 0.20 |
| Parvimonas | 0.47 | 0.62 | 0.25 | 0.53 |
| Oribacterium | 0.42 | 0.32 | 0.26 | 0.33 |
| Bifidobacterium | 0.42 | 0.10 | 0.04 | 0.18 |
| Legionella | 0.37 | 0.79 | 0.43 | 0.41 |
| Corynebacterium | 0.35 | 3.32 | 2.74 | 0.31 |
| Mogibacterium | 0.30 | 0.25 | 0.11 | 0.30 |
| Lachnoanaerobaculum | 0.26 | 0.24 | 0.10 | 0.14 |
| Abiotrophia | 0.22 | 0.31 | 0.85 | 0.57 |
| Scardovia | 0.20 | 2.01 | 1.67 | 0.10 |
| Campylobacter | 0.17 | 0.52 | 0.69 | 0.24 |
| Filifactor | 0.15 | 0.18 | 0.08 | 0.18 |
| Dialister | 0.13 | 0.82 | 1.23 | 0.26 |
| Tannerella | 0.11 | 0.14 | 0.06 | 0.12 |
| Bulleidia | 0.11 | 0.10 | 0.05 | 0.09 |
| Treponema | 0.11 | 0.50 | 0.51 | 0.12 |
| Slackia | 0.08 | 0.09 | 0.03 | 0.17 |
| Lautropia | 0.07 | 0.21 | 1.92 | 2.31 |
| Kingella | 0.07 | 0.14 | 0.41 | 0.05 |
| Alloscardovia | 0.06 | 0.00 | 0.00 | 0.04 |
| Bacteroidetes | 0.06 | 0.18 | 0.05 | 0.10 |
| Aggregatibacter | 0.06 | 0.14 | 0.22 | 0.08 |
| Selenomonas | 0.06 | 0.34 | 0.24 | 0.20 |
| Eikenella | 0.06 | 0.21 | 0.35 | 0.05 |
| Lactobacillus | 0.06 | 0.09 | 0.09 | 1.54 |
| Actinobacillus_unclassified | 0.06 | 0.18 | 0.15 | 0.04 |
| candidate_division_TM7 | 0.05 | 0.53 | 0.34 | 0.05 |
| Olsenella | 0.05 | 0.06 | 0.03 | 0.05 |
| Eggerthia_catenaformis | 0.04 | 0.04 | 0.00 | 0.02 |
| Shuttleworthia | 0.03 | 0.05 | 0.11 | 0.03 |
| Cardiobacterium | 0.03 | 0.11 | 0.24 | 0.04 |
| Catonella | 0.02 | 0.06 | 0.08 | 0.02 |
| Actinobaculum | 0.02 | 0.54 | 1.60 | 0.07 |
| Simonsiella | 0.02 | 0.01 | 0.00 | 0.00 |
| Anaerococcus | 0.01 | 0.08 | 0.11 | 0.00 |
| Propionibacterium | 0.01 | 0.12 | 0.15 | 0.20 |
| Mitsuokella | 0.01 | 0.01 | 0.00 | 0.00 |
| Riemerella_unclassified | 0.01 | 0.01 | 0.02 | 0.01 |
| Peptoniphilus | 0.01 | 0.00 | 0.00 | 0.00 |
| Pseudoramibacter | 0.00 | 0.00 | 0.00 | 0.01 |
| Desulfobulbus | 0.00 | 0.02 | 0.02 | 0.00 |
| Staphylococcus | 0.00 | 0.03 | 0.00 | 0.00 |
| Eubacteriaceae | 0.00 | 0.01 | 0.00 | 0.01 |
| Gardnerella | 0.00 | 0.01 | 0.00 | 0.00 |
| Centipeda | 0.00 | 0.00 | 0.00 | 0.00 |
| Parascardovia | 0.00 | 0.52 | 0.12 | 0.02 |
| Mycoplasma | 0.00 | 0.00 | 0.00 | 0.00 |
| Pseudomonas | 0.00 | 0.00 | 0.00 | 0.46 |
| Ruminococcus_torques | 0.00 | 0.00 | 0.00 | 0.00 |
| Xanthomonas | 0.00 | 0.00 | 0.00 | 0.00 |
| Desulfovibrio | 0.00 | 0.00 | 0.00 | 0.00 |
| Johnsonella | 0.00 | 0.03 | 0.00 | 0.00 |

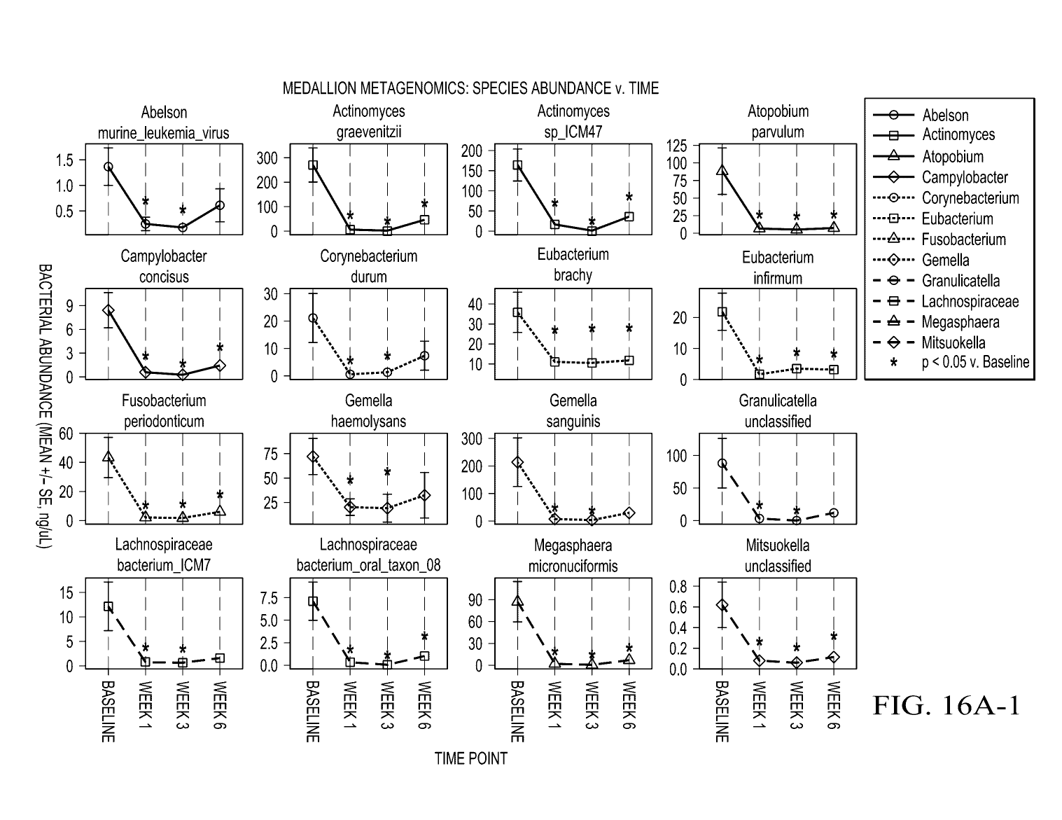

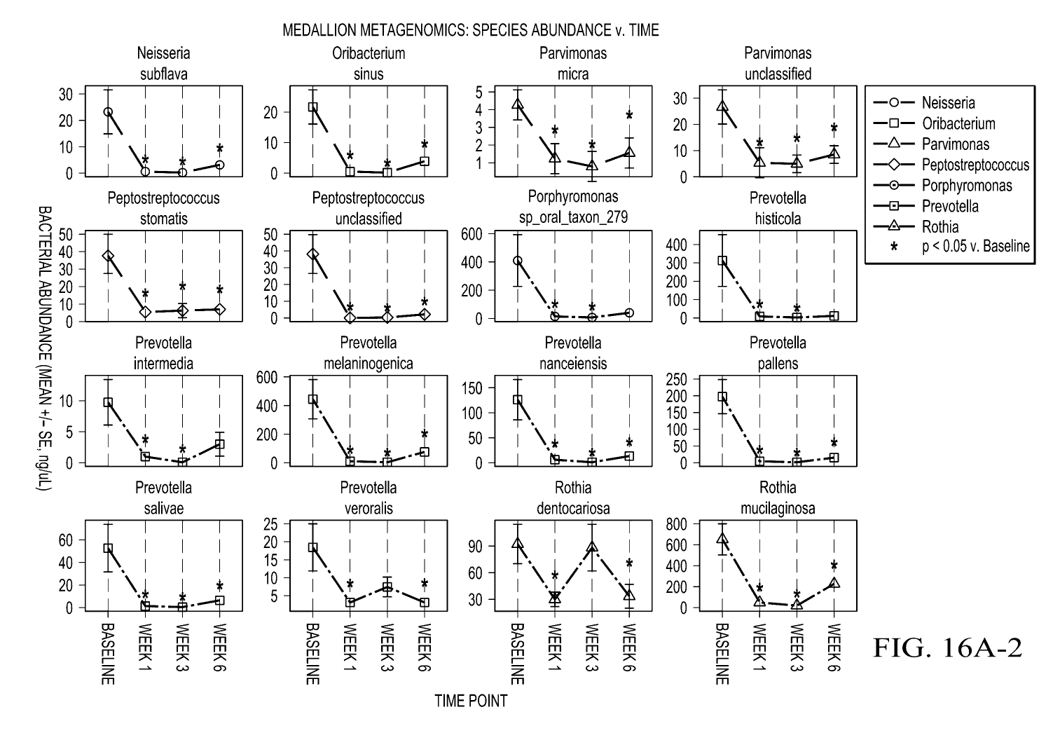

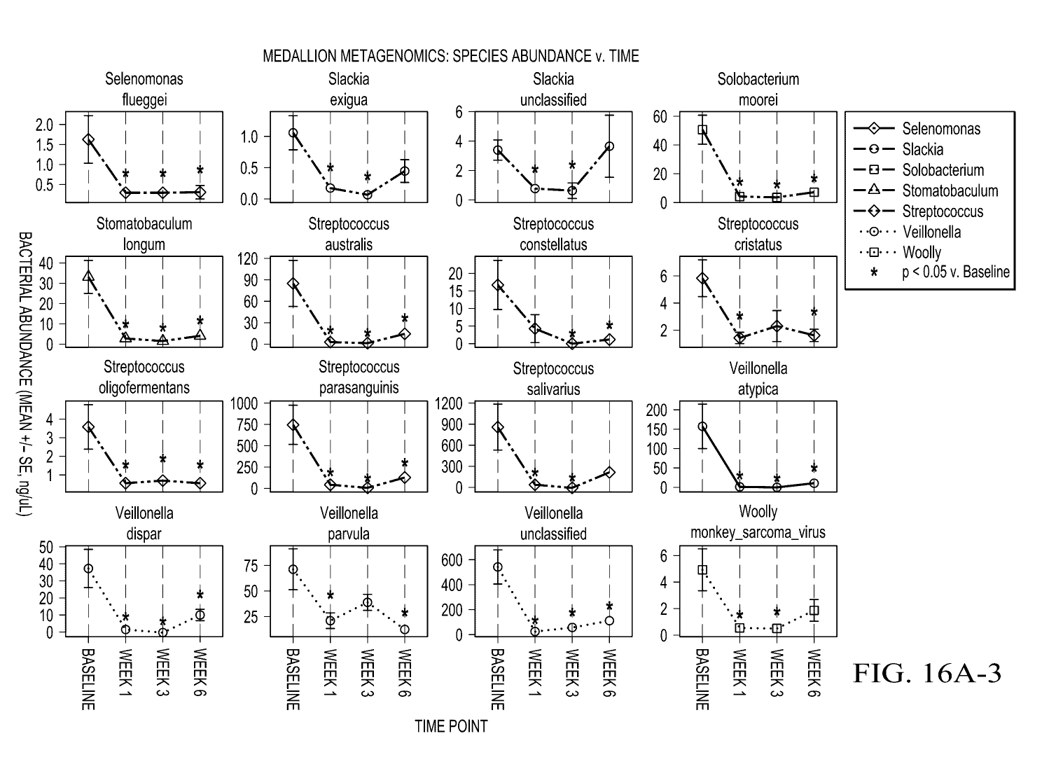

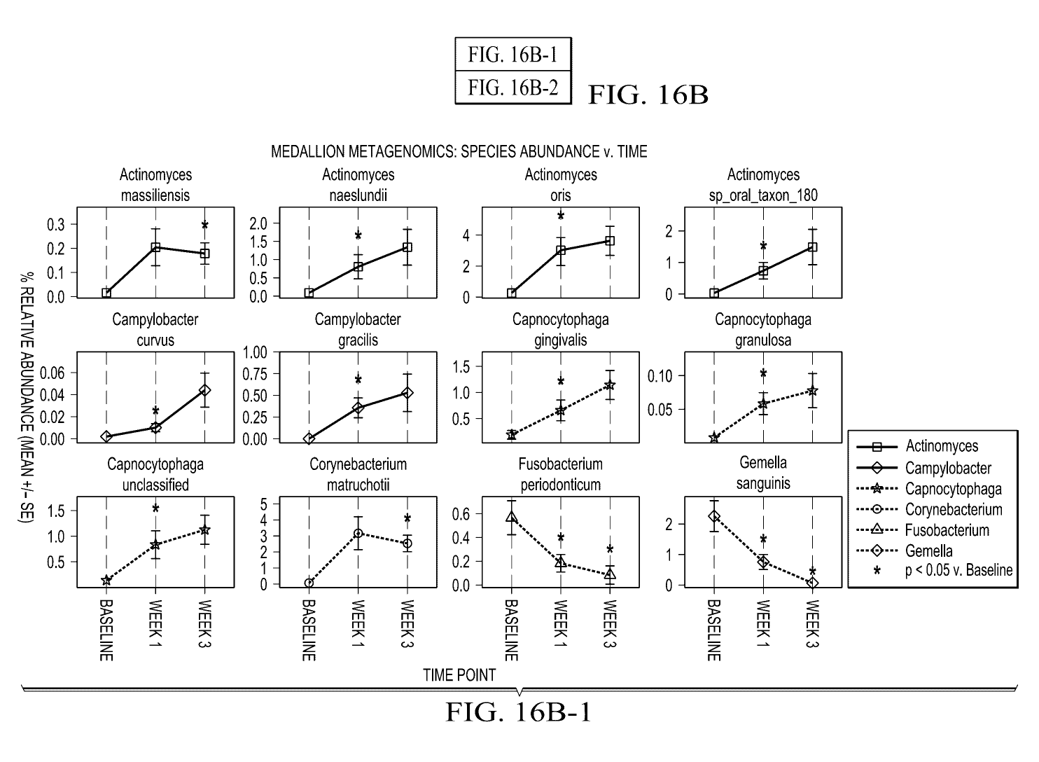

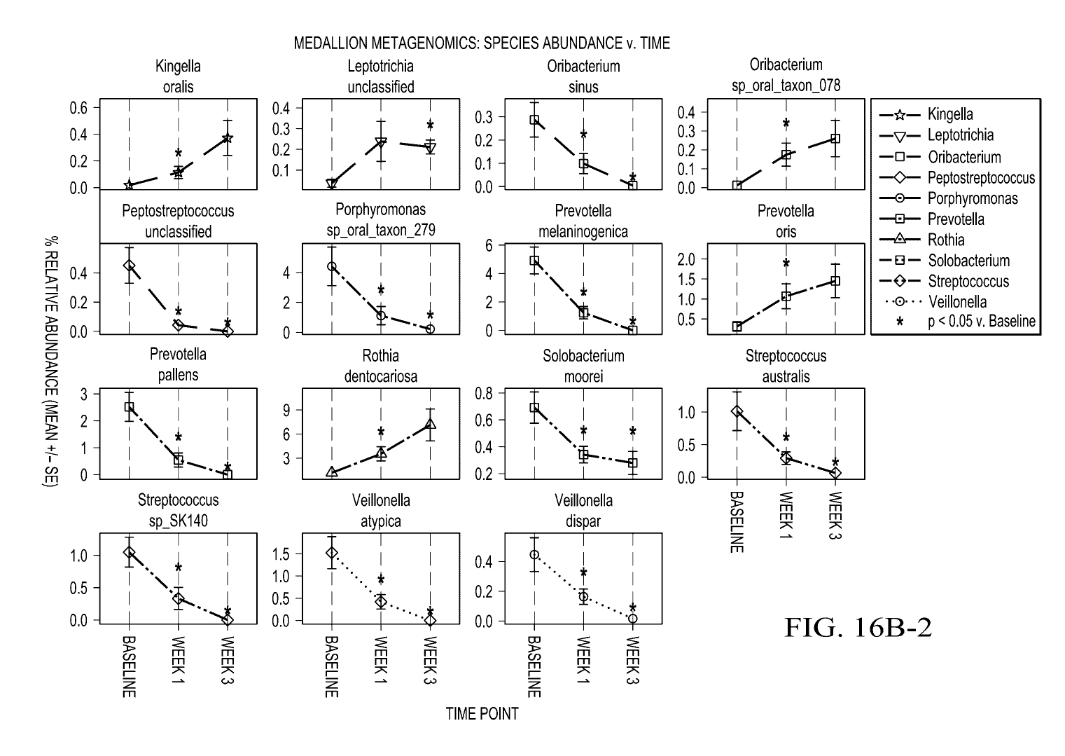

The abundance of certain bacteria in the supragingival plaques changed in the six week treatments as shown in Table 6B and FIG. 16A. Certain bacteria were decreased either in weeks 1, 3 or 6. Those microbes comprise Actinomyces_graevenitzii, Actinomyces_massiliensis, Actinomyces_sp_ICM47, Atopobium_parvulum, Campylobacter_concisus, Catonella_morbi, Eubacterium_brachy, Eubacterium_infirmum, Fusobacterium_nucleatum, Fusobacterium_periodonticum, Lachnoanaerobaculum_saburreum, Lachnospiraceae_bacterium_oral_taxon_082, Megasphaera_micronuciformis, Mitsuokella_unclassified, Neisseria_subflava, Oribacterium_sinus, Parvimonas_micra, Parvimonas_unclassified, Peptostreptococcus_stomatis, Peptostreptococcus_unclassified, Pestivirus_Giraffe_1, Porphyromonas_endodontalis, Porphyromonas_sp_oral_taxon_279, Prevotella_histicola, Prevotella_maculosa, Prevotella_melaninogenica, Prevotella_nanceiensis, Prevotella_oris, Prevotella pallens, Prevotella_salivae, Prevotella_sp_C561, Prevotella_veroralis, Rothia_dentocariosa, Rothia_mucilaginosa, Selenomonas_flueggei, Selenomonas_sputigena, Solobacterium_moorei, Stomatobaculum_longum, Streptococcus_anginosus, Streptococcus_australis, Streptococcus_constellatus, Streptococcus_cristatus, Streptococcus_oligofermentans, Streptococcus_parasanguinis, Streptococcus_sanguinis, Veillonella_atypica, Veillonella_dispar, Veillonella_parvula, Veillonella_unclassified. The amount of each bacterial species was plotted over the four time periods of the treatment. The amount of certain bacteria, such as Peptostreptococcus stomatis and Prevotella intermedia, was reduced during the six week of treatment as shown in FIG. 16A. The relative amount of each bacterial species was plotted over the three time periods of the treatment (FIG. 16B). The relative amount of certain bacteria, such as Actinomyces massiliensis, actinomyces naslundii, capnocytophaga gingivalis, and campylobacter gracilis, was increased, while the abundance of Prevotella pallens, Prevetella malaninogenica, and Porphyromonas sp_oral_taxon_279 was reduced during the three weeks of treatment.

TABLE 6B
Decrease of gingivitis-associated bacteria in supragingival plaques during the
period of 6-week regimen treatment.

| | | | | | p. value | p. value | p. value |
|---|---|---|---|---|---|---|---|
| | | | | | Week 1 v. | Week 3 v. | Week 6 v. |
| | Relative | Relative | Relative | Relative | Baseline - | Baseline - | Baseline - |
| | Abundance | Abundance | Abundance | Abundance | Relative | Relative | Relative |
| Species | Baseline | Week 1 | Week 3 | Week 6 | Abundance | Abundance | Abundance |
| Actinomyces_graevenitzii | 3.61 | 1.2 | 0.0016 | 2.18 | 0.125 | 0.0198 | 0.302 |
| Actinomyces_massiliensis | 0.0147 | 0.203 | 0.176 | 0.0365 | 0.026 | 0.00739 | 0.0885 |
| Actinomyces_sp_ICM47 | 2.69 | 1.36 | 0 | 1.82 | 0.289 | 0.00982 | 0.547 |
| Atopobium_parvulum | 1.02 | 0.738 | 0.309 | 0.379 | 0.43 | 0.0199 | 0.0375 |
| Campylobacter_concisus | 0.0966 | 0.0544 | 0.0092 | 0.0664 | 0.19 | 0.0015 | 0.404 |
| Catonella_morbi | 0.0241 | 0.0592 | 0.0755 | 0.0219 | 0.108 | 0.187 | 0.972 |
| Eubacterium_brachy | 0.619 | 0.792 | 0.592 | 0.587 | 0.604 | 0.935 | 0.904 |
| Eubacterium_infirmum | 0.298 | 0.141 | 0.267 | 0.127 | 0.122 | 0.816 | 0.112 |
| Fusobacterium_nucleatum | 0.282 | 1.21 | 1.07 | 0.31 | 0.0361 | 0.0688 | 0.785 |
| Fusobacterium_periodonticum | 0.567 | 0.186 | 0.0856 | 0.244 | 0.0268 | 0.00707 | 0.101 |
| Lachnoanaerobaculum_saburreum | 0.0116 | 0.12 | 0.0635 | 0.0106 | 0.115 | 0.118 | 0.632 |
| Lachnospiraceae_bacterium_oral_taxon_082 | 0.0974 | 0.0605 | 0.00232 | 0.0427 | 0.296 | 0.00055 | 0.0883 |
| Megasphaera_micronuciformis | 1.1 | 0.347 | 0.0134 | 0.295 | 0.0703 | 0.0108 | 0.0565 |
| Mitsuokella_unclassified | 0.00705 | 0.0071 | 0.00377 | 0.00461 | 0.988 | 0.27 | 0.382 |
| Neisseria_subflava | 0.374 | 0.0431 | 0.018 | 0.178 | 0.0615 | 0.0446 | 0.331 |
| Oribacterium_sinus | 0.29 | 0.101 | 0.00147 | 0.176 | 0.0371 | 0.00144 | 0.284 |
| Parvimonas_micra | 0.0686 | 0.107 | 0.0485 | 0.075 | 0.412 | 0.481 | 0.69 |
| Parvimonas_unclassified | 0.393 | 0.507 | 0.2 | 0.443 | 0.638 | 0.163 | 0.65 |
| Peptostreptococcus_stomatis | 0.54 | 0.38 | 0.231 | 0.43 | 0.39 | 0.101 | 0.779 |
| Peptostreptococcus_unclassified | 0.449 | 0.0396 | 0.00028 | 0.109 | 0.00428 | 0.00217 | 0.0146 |
| Pestivirus_Giraffe_1 | 0.00083 | 0.00146 | 0.00066 | 0.00221 | 0.478 | 0.814 | 0.0952 |
| Porphyromonas_endodontalis | 0.264 | 0.197 | 0.301 | 0.479 | 0.425 | 0.808 | 0.0902 |
| Porphyromonas_sp_oral_taxon_279 | 4.39 | 1.15 | 0.284 | 1.86 | 0.0276 | 0.00497 | 0.0977 |
| Prevotella_histicola | 2.77 | 0.851 | 0.00025 | 0.721 | 0.109 | 0.0119 | 0.0666 |
| Prevotella_maculosa | 0.0148 | 0.133 | 0.15 | 0.0105 | 0.0413 | 0.0912 | 0.705 |
| Prevotella_melaninogenica | 4.9 | 1.24 | 0.0236 | 4.19 | 0.00246 | 0.00017 | 0.895 |
| Prevotella_nanceiensis | 1.61 | 0.646 | 0.0228 | 0.835 | 0.0818 | 0.00129 | 0.144 |
| Prevotella_oris | 0.319 | 1.07 | 1.46 | 0.211 | 0.0358 | 0.0307 | 0.434 |
| Prevotella_pallens | 2.53 | 0.553 | 0.00042 | 0.712 | 0.00322 | 0.0003 | 0.00705 |
| Prevotella_salivae | 0.501 | 0.179 | 0.0147 | 0.312 | 0.0528 | 0.00298 | 0.332 |
| Prevotella_sp_C561 | 1.47 | 0.295 | 0 | 0.232 | 0.0792 | 0.0263 | 0.0655 |
| Prevotella_veroralis | 0.257 | 0.282 | 0.541 | 0.181 | 0.871 | 0.363 | 0.694 |
| Rothia_dentocariosa | 1.18 | 3.53 | 7.2 | 1.87 | 0.02 | 0.022 | 0.183 |
| Rothia_mucilaginosa | 8.35 | 7.28 | 1.24 | 12.7 | 0.731 | 3.8E−05 | 0.0299 |
| Selenomonas_flueggei | 0.0179 | 0.0264 | 0.0361 | 0.0107 | 0.342 | 0.362 | 0.4 |
| Selenomonas_sputigena | 0.0121 | 0.0314 | 0.0271 | 0.00853 | 0.116 | 0.145 | 0.558 |
| Solobacterium_moorei | 0.693 | 0.341 | 0.279 | 0.357 | 0.0141 | 0.01 | 0.0303 |
| Stomatobaculum_longum | 0.476 | 0.318 | 0.056 | 0.197 | 0.335 | 0.00305 | 0.0518 |
| Streptococcus_anginosus | 0.226 | 0.819 | 0.44 | 0.211 | 0.143 | 0.414 | 0.947 |
| Streptococcus_australis | 1.02 | 0.288 | 0.0591 | 0.784 | 0.031 | 0.00575 | 0.684 |
| Streptococcus_constellatus | 0.31 | 0.38 | 0.00031 | 0.0929 | 0.825 | 0.116 | 0.302 |
| Streptococcus_cristatus | 0.0873 | 0.127 | 0.122 | 0.0705 | 0.311 | 0.567 | 0.496 |
| Streptococcus_oligofermentans | 0.0518 | 0.0389 | 0.0273 | 0.018 | 0.518 | 0.194 | 0.0562 |
| Streptococcus_parasanguinis | 7.99 | 4.46 | 0.285 | 7.68 | 0.132 | 0.00117 | 0.961 |
| Streptococcus_sanguinis | 0.602 | 1.22 | 2.82 | 1.48 | 0.085 | 0.0675 | 0.0218 |
| Veillonella_atypica | 1.52 | 0.413 | 0.00603 | 0.696 | 0.0118 | 0.0009 | 0.0988 |
| Veillonella_dispar | 0.448 | 0.164 | 0.0159 | 0.418 | 0.0356 | 0.002 | 0.811 |
| Veillonella_parvula | 0.772 | 1.88 | 2.7 | 0.517 | 0.0741 | 0.0102 | 0.161 |
| Veillonella_unclassified | 6.08 | 2.69 | 4.14 | 4.76 | 0.00212 | 0.181 | 0.146 |
| | | | | | p. value | p. value | p. value |
| | Bacterial | Bacterial | Bacterial | Bacterial | Week 1 v. | Week 3 v. | Week 6 v. |
| | Abundance | Abundance | Abundance | Abundance | Baseline - | Baseline - | Baseline - |
| | (ng/uL) - | (ng/uL) - | (ng/uL) - | (ng/uL) - | Bacterial | Bacterial | Bacterial |
| Species | Baseline | Week 1 | Week 3 | Week 6 | Abundance | Abundance | Abundance |
| Actinomyces_graevenitzii | 270 | 9.95 | 0.0425 | 45.3 | 0.00266 | 0.00201 | 0.00735 |
| Actinomyces_massiliensis | 1.21 | 3.28 | 2.51 | 0.906 | 0.378 | 0.185 | 0.595 |
| Actinomyces_sp_ICM47 | 166 | 17 | 0 | 35.1 | 0.00276 | 0.00106 | 0.00669 |
| Atopobium_parvulum | 88.9 | 6.85 | 5.47 | 8.28 | 0.0261 | 0.0241 | 0.0284 |
| Campylobacter_concisus | 8.42 | 0.661 | 0.208 | 1.38 | 0.00326 | 0.00212 | 0.0066 |
| Catonella_morbi | 1.7 | 0.977 | 1.76 | 0.511 | 0.329 | 0.962 | 0.0475 |
| Eubacterium_brachy | 35.9 | 10.8 | 10.2 | 11.6 | 0.0397 | 0.0421 | 0.048 |
| Eubacterium_infirmum | 22 | 1.61 | 3.13 | 2.88 | 0.00434 | 0.00733 | 0.00671 |
| Fusobacterium_nucleatum | 21 | 20.9 | 17.8 | 6.96 | 0.993 | 0.698 | 0.0129 |
| Fusobacterium_periodonticum | 44.2 | 1.86 | 1.63 | 5.61 | 0.00702 | 0.00684 | 0.0129 |
| Lachnoanaerobaculum_saburreum | 0.952 | 2.89 | 0.966 | 0.285 | 0.422 | 0.984 | 0.0317 |
| Lachnospiraceae_bacterium_oral_taxon_082 | 7.05 | 0.394 | 0.0477 | 1.07 | 0.00804 | 0.00582 | 0.0163 |
| Megasphaera_micronuciformis | 86.2 | 2.98 | 0.305 | 6.39 | 0.00923 | 0.0076 | 0.0118 |
| Mitsuokella_unclassified | 0.621 | 0.0852 | 0.0631 | 0.118 | 0.032 | 0.0257 | 0.0434 |
| Neisseria_subflava | 23.3 | 0.57 | 0.327 | 3.09 | 0.0161 | 0.0152 | 0.0299 |
| Oribacterium_sinus | 21.7 | 1.19 | 0.0294 | 3.74 | 0.00289 | 0.00191 | 0.0074 |
| Parvimonas_micra | 4.27 | 1.25 | 0.776 | 1.55 | 0.00609 | 0.00139 | 0.0277 |
| Parvimonas_unclassified | 26.6 | 5.36 | 4.69 | 8.46 | 0.00696 | 0.00751 | 0.0233 |
| Peptostreptococcus_stomatis | 39.1 | 5.42 | 6.17 | 7.11 | 0.01 | 0.0131 | 0.0136 |
| Peptostreptococcus_unclassified | 37.4 | 0.358 | 0.00485 | 2.38 | 0.00571 | 0.00537 | 0.00817 |
| Pestivirus_Giraffe_1 | 0.0653 | 0.0158 | 0.00479 | 0.041 | 0.192 | 0.104 | 0.544 |
| Porphyromonas_endodontalis | 19.7 | 2.33 | 6.48 | 7.69 | 0.00853 | 0.0745 | 0.0748 |
| Porphyromonas_sp_oral_taxon_279 | 402 | 13.3 | 5.5 | 32.8 | 0.0497 | 0.0458 | 0.0608 |
| Prevotella_histicola | 312 | 7.36 | 0.00819 | 15.5 | 0.047 | 0.0425 | 0.0524 |
| Prevotella_maculosa | 1.3 | 2.11 | 1.82 | 0.231 | 0.525 | 0.517 | 0.0289 |
| Prevotella_melaninogenica | 447 | 10.3 | 0.488 | 76.7 | 0.00692 | 0.00601 | 0.019 |
| Prevotella_nanceiensis | 126 | 7.55 | 0.532 | 14.2 | 0.00985 | 0.00678 | 0.0136 |
| Prevotella_oris | 25.1 | 16.8 | 18.4 | 4.41 | 0.508 | 0.496 | 0.0219 |
| Prevotella_pallens | 196 | 3.94 | 0.00783 | 16.3 | 0.00169 | 0.00145 | 0.0028 |
| Prevotella_salivae | 52.7 | 1.45 | 0.117 | 6.26 | 0.0288 | 0.0254 | 0.0449 |
| Prevotella_sp_C561 | 144 | 1.17 | 0 | 6.8 | 0.0959 | 0.0935 | 0.109 |
| Prevotella_veroralis | 18.6 | 3.14 | 7.56 | 3.27 | 0.0333 | 0.133 | 0.0348 |
| Rothia_dentocariosa | 92.5 | 29.6 | 88.3 | 33.9 | 0.0162 | 0.903 | 0.0324 |
| Rothia_mucilaginosa | 651 | 53.9 | 16.5 | 219 | 0.0009 | 0.00054 | 0.0103 |
| Selenomonas_flueggei | 1.63 | 0.306 | 0.293 | 0.31 | 0.0469 | 0.0451 | 0.0495 |
| Selenomonas_sputigena | 0.848 | 0.586 | 0.437 | 0.227 | 0.571 | 0.15 | 0.022 |
| Solobacterium_moorei | 50.7 | 4.23 | 3.55 | 6.75 | 0.00028 | 0.00025 | 0.00046 |
| Stomatobaculum_longum | 33.2 | 3.03 | 1.41 | 4.29 | 0.00231 | 0.00156 | 0.00318 |
| Streptococcus_anginosus | 14.3 | 9.14 | 9.94 | 3.15 | 0.379 | 0.62 | 0.00894 |
| Streptococcus_australis | 84.8 | 2.79 | 0.983 | 14.2 | 0.0234 | 0.021 | 0.0468 |
| Streptococcus_constellatus | 16.6 | 4.39 | 0.0059 | 1.13 | 0.138 | 0.0327 | 0.0447 |
| Streptococcus_cristatus | 5.84 | 1.46 | 2.3 | 1.67 | 0.00594 | 0.0585 | 0.00838 |
| Streptococcus_oligofermentans | 3.59 | 0.514 | 0.665 | 0.506 | 0.0212 | 0.029 | 0.0211 |
| Streptococcus_parasanguinis | 748 | 41.2 | 3.81 | 129 | 0.00773 | 0.00554 | 0.0168 |
| Streptococcus_sanguinis | 43 | 15.5 | 49.5 | 37.1 | 0.0373 | 0.76 | 0.709 |
| Veillonella_atypica | 157 | 2.96 | 0.135 | 15.4 | 0.0184 | 0.0167 | 0.0283 |
| Veillonella_dispar | 37.3 | 1.75 | 0.254 | 9.91 | 0.00689 | 0.00526 | 0.0317 |
| Veillonella_parvula | 71.1 | 20.8 | 38.9 | 12.9 | 0.0306 | 0.153 | 0.0118 |
| Veillonella_unclassified | 548 | 23.4 | 56.6 | 107 | 0.00213 | 0.00345 | 0.00711 |

### Example 15—Production of Cytokines, Chemokines and Other Bioactive Proteins Decreased as Gingivitis Symptoms were Alleviated During 6 Week of Treatment

In the same clinical study as described in EXAMPLE 14, gingival-brush samples were collected from the same panelists as in EXAMPLE 14. Before sampling, panelists rinsed their mouths for 30 seconds with water. A dental hygienist then sampled the area just above the gumline using a buccal swab brush (Epicentre Biotechnologies cat.# MB100SP). The swab was immediately placed into 1 ml extraction buffer [PBS, 0.25M NaCl, 1× Halt™ Protease Inhibitor Single-Use Cocktail (Lifetechnologies, Grand Island, N.Y.)] in a 1.5 ml Eppendorf tube vortexed for 30 seconds, and immediately frozen on dry ice and stored in a −80 C freezer until analysis. The samples were taken out of the freezer, thawed and extracted by placing the samples on a tube shaker for 30 minutes at 4° C. The tubes were centrifuged at 15000 RPM for 10 min in Eppendorf Centrifuge 5417R (Eppendorf, Ontario, Canada) to pellet any debris. The extract (800 μl) was analyzed for protein concentrations using the Bio-Rad protein assay (BioRad, Hercules, Calif.).

Forty proteins were measured in the gingival samples using V-PLEX Human Biomarker 40-Plex Kit (Meso Scale Diagnostics, Rockville, Md.). The assay was performed following the manufacturer's instruction.

Among the proteins measured in the gingival samples, most proteins in the Proinflammatory Panel 1 (human), Cytokine Panel 1 (human), Chemokine Panel 1 (human), Angiogenesis Panel 1 (human), and Vascular Injury Panel 2 (human) had significant changes in their abundance during the 6-week treatment (TABLE 6C). Those include FN-γ, IL-1β, IL-2, IL-4, IL-6, IL-8, IL-10, IL-12p70, IL-13, TNF-α, GM-CSF, IL-5, IL-16, IL-7, IL-12/IL-23p40, IL-la, VEGF-A, IL-17A, IL-15, TNF-J3, IL-8 (HA), MCP-1, MCP-4, Eotaxin, IP-10, MDC, Eotaxin-3, TARC, MIP-1α, MIP-1β, VEGF-C, VEGF-D, Tie-2, Flt-1/VEGFR1, P1GF, FGF (basic), SAA, CRP, VCAM-1, and ICAM-1.

TABLE 6C
Changes in abundance of proteins in the gingival-brush samples.

| | Mean | | | | α = 0.05 | | | |
|---|---|---|---|---|---|---|---|---|
| | Baseline | Week 1 | Week 3 | Week 6 | Baseline | Week 1 | Week 3 | Week 6 |
| ICAM-1 | 16.035 | 12.209 | 10.090 | 9.767 | A | B | B, C | C |
| IL-1α | 3.554 | 2.331 | 2.181 | 1.891 | A | A, B | B, C | C |
| IL-1β | 53.666 | 35.575 | 24.295 | 24.440 | A | B | C | C |
| TNF-β | 0.0013 | 0.0010 | 0.0008 | 0.0007 | A | B | C | C |
| IL-12p70 | 0.172 | 0.148 | 0.118 | 0.127 | A | A, B | C | B, C |
| IL-13 | 0.805 | 0.762 | 0.624 | 0.648 | A | A, B | C | B, C |
| IL-4 | 0.127 | 0.115 | 0.090 | 0.096 | A | A, B | C | B, C |
| IL-5 | 0.004 | 0.003 | 0.002 | 0.003 | A | B | C | B, C |
| CRP | 15.637 | 12.743 | 12.385 | 5.809 | A | A | A | B |
| Eotaxin | 0.077 | 0.064 | 0.059 | 0.059 | A | A, B | B | B |
| GM-CSF | 0.010 | 0.008 | 0.008 | 0.008 | A | B | B | B |
| IFNγ | 0.530 | 0.446 | 0.378 | 0.386 | A | A, B | B | B |
| IL-10 | 0.875 | 0.490 | 0.423 | 0.244 | A | A, B | B | B |
| IL-15 | 0.005 | 0.003 | 0.003 | 0.003 | A | B | B | B |
| IL-16 | 0.466 | 0.345 | 0.342 | 0.295 | A | B | B | B |
| IL-6 | 0.196 | 0.192 | 0.168 | 0.150 | A | A | A, B | B |
| IL-7 | 0.004 | 0.003 | 0.003 | 0.003 | A | B | B | B |
| IL-8 | 856.276 | 652.066 | 567.361 | 572.602 | A | B | B | B |
| MCP-1 | 0.053 | 0.047 | 0.039 | 0.039 | A | A, B | B | B |
| MDC | 0.399 | 0.407 | 0.345 | 0.339 | A | A | B | B |
| SAA | 7.039 | 6.905 | 6.092 | 5.162 | A | A | A, B | B |
| Tie-2 | 0.273 | 0.239 | 0.267 | 0.221 | A | A, B | A | B |
| VCAM-1 | 4.971 | 3.706 | 3.156 | 2.892 | A | B | B | B |
| VEGF | 0.625 | 0.511 | 0.478 | 0.480 | A | B | B | B |
| VEGF 2 | 0.772 | 0.661 | 0.620 | 0.582 | A | B | B | B |
| VEGF-D | 0.057 | 0.052 | 0.051 | 0.045 | A | A, B | A, B | B |
| VEGF-C | 0.145 | 0.149 | 0.125 | 0.137 | A, B | A | B | A, B |
| TARC | 0.020 | 0.029 | 0.019 | 0.019 | A | B | A | A |
| bFGF | 0.020 | 0.015 | 0.012 | 0.013 | A | A | A | A |
| Eotaxin-3 | 0.095 | 0.108 | 0.091 | 0.094 | A | A | A | A |
| Fk-1 | 0.390 | 0.518 | 0.433 | 0.415 | A | B | A, B | A |
| IL-12p40 | 0.039 | 0.031 | 0.028 | 0.031 | A | A | A | A |
| IL-2 | 0.166 | 0.199 | 0.210 | 0.162 | A | A | A | A |
| IL-8 (HA) | 47.508 | 44.362 | 41.260 | 39.119 | A | A | A | A |
| IP-10 | 0.540 | 1.688 | 0.740 | 0.606 | A | A | A | A |
| MCP-4 | 0.023 | 0.023 | 0.020 | 0.022 | A | A | A | A |
| MIP-1α | 0.091 | 0.091 | 0.084 | 0.080 | A | A | A | A |
| MIP-1β | 0.091 | 0.100 | 0.110 | 0.094 | A | A | A | A |
| TNFα | 2.009 | 2.067 | 2.021 | 1.670 | A | A | A | A |

### Example 16—One Hundred Seventy Metabolites were Identified in Gingival Samples

The same gingival-brush samples as described in EXAMPLE 15 were used for metabonomic analyses. Fourteen panelists were selected randomly from each treatment group to determine if any metabolite concentrations were changed in gingival samples during the first 3 weeks of treatment. Both baseline and week 3 samples were sent to Metabolon, Inc. (Durham, N.C.) for metabonomic measurement. 170 metabolites were identified and quantified. As shown in TABLE 7, some metabolite concentrations were changed during the first 3 weeks of treatment. Citrulline concentrations in the gingival samples were reduced after three weeks of treatment in the treatment regimen group. Similarly, ornithine was also reduced in the treatment regimen group after three weeks of treatment. Reduction of citrulline and ornithine was likely associated with alleviation of gingivitis.

TABLE 7
Comparison of metabolites in gingival brush samples between
baseline and week 3 during gingivitis treatment.

| | Baseline | 3 week | | | | |
|---|---|---|---|---|---|---|
| Biochemical Name | mean | mean | 3 week/baseline | p-value | q-value | Mass |
| deoxycarnitine | 3.2381 | 0.6088 | 0.19 | 0.0003 | 0.0168 | 146.1 |
| proline | 1.405 | 0.99 | 0.7 | 0.0033 | 0.0546 | 116.1 |
| 2-methylbutyrylcarnitine (C5) | 1.7769 | 0.6997 | 0.39 | 0.0034 | 0.0546 | 246.1 |
| lysylleucine | 1.2009 | 0.5275 | 0.44 | 0.0036 | 0.0546 | 260.2 |
| threonylleucine | 1.1513 | 0.4931 | 0.43 | 0.0044 | 0.0594 | 231.2 |
| trehalose | 2.3563 | 0.9084 | 0.39 | 0.0054 | 0.0647 | 361.2 |
| arginylleucine | 1.4447 | 0.6819 | 0.47 | 0.0084 | 0.0777 | 288.3 |
| lysylphenylalanine | 1.1682 | 0.4563 | 0.39 | 0.0095 | 0.0777 | 294.3 |
| citrulline | 1.0147 | 0.71 | 0.7 | 0.0104 | 0.0777 | 176.1 |
| arginylphenylalanine | 0.9616 | 0.3335 | 0.35 | 0.0119 | 0.0777 | 322.2 |
| pyroglutamine* | 1.3424 | 0.7873 | 0.59 | 0.0136 | 0.0777 | 129.2 |
| EDTA | 1.5985 | 0.8384 | 0.52 | 0.0138 | 0.0777 | 291.1 |
| tyrosine | 1.383 | 1.0299 | 0.74 | 0.0161 | 0.0787 | 182.1 |
| propionylcarnitine | 1.2565 | 0.7688 | 0.61 | 0.0201 | 0.0877 | 218.2 |
| phosphate | 1.4008 | 0.8376 | 0.6 | 0.0208 | 0.0877 | 298.9 |
| maltose | 0.8727 | 1.4481 | 1.66 | 0.022 | 0.0877 | 204.1 |
| threonylphenylalanine | 1.7693 | 0.918 | 0.52 | 0.0233 | 0.0877 | 267.2 |
| leucylleucine | 0.9505 | 0.4393 | 0.46 | 0.0251 | 0.0877 | 245.1 |
| adenosine 5′-monophosphate (AMP) | 1.4092 | 0.8451 | 0.6 | 0.0295 | 0.0956 | 348.1 |
| valine | 1.1598 | 0.9271 | 0.8 | 0.0304 | 0.0956 | 118.1 |
| alanine | 0.8721 | 1.102 | 1.26 | 0.0318 | 0.0973 | 115.9 |
| ornithine | 1.2916 | 0.7069 | 0.55 | 0.0367 | 0.1035 | 141.9 |
| glutamine | 1.2459 | 0.8366 | 0.67 | 0.0374 | 0.1035 | 147.2 |
| 1- | 1.2294 | 0.8274 | 0.67 | 0.038 | 0.1035 | 500.3 |
| arachidonoylglycerophosphoethanolamine | | | | | | |
| glycerol | 1.3783 | 0.8308 | 0.6 | 0.0391 | 0.1035 | 205 |
| lauryl sulfate | 1.685 | 0.8623 | 0.51 | 0.0397 | 0.1035 | 265.2 |
| mannitol | 1.3004 | 0.7982 | 0.61 | 0.042 | 0.107 | 319.1 |
| tryptophan | 1.1518 | 0.9089 | 0.79 | 0.0487 | 0.1185 | 205.1 |
| valylvaline | 0.9347 | 0.8231 | 0.88 | 0.0508 | 0.1207 | 215.2 |
| erythritol | 1.625 | 0.8085 | 0.5 | 0.0582 | 0.1325 | 217 |
| 13-HODE + 9-HODE | 1.0877 | 0.7088 | 0.65 | 0.0601 | 0.1338 | 295.2 |
| leucine | 1.2158 | 0.9359 | 0.77 | 0.0613 | 0.1338 | 132.2 |
| spermidine | 1.1613 | 0.8678 | 0.75 | 0.0687 | 0.1465 | 146.2 |
| asparagylleucine | 0.9295 | 0.6122 | 0.66 | 0.0698 | 0.1465 | 246.2 |
| succinate | 1.2929 | 0.8113 | 0.63 | 0.0754 | 0.1548 | 247 |
| 1-oleoylglycerophosphoethanolamine | 0.7378 | 1.0747 | 1.46 | 0.0767 | 0.1548 | 478.3 |
| palatinitol | 1.4395 | 0.8272 | 0.57 | 0.0782 | 0.1549 | 204 |
| serylisoleucine* | 1.1753 | 0.7169 | 0.61 | 0.0814 | 0.1583 | 219.2 |
| fructose | 1.9933 | 1.1106 | 0.56 | 0.0847 | 0.1605 | 217 |
| maltotriose | 1.0456 | 1.8347 | 1.75 | 0.0858 | 0.1605 | 204 |
| glutathione, oxidized (GSSG) | 1.0161 | 1.4669 | 1.44 | 0.087 | 0.1605 | 613.1 |

### Example 17—Citrulline was Reduced in Regimen Treatment Over 6 Weeks

Quantitation of citrulline and ornithine from the extracts of the Gingival-brush samples was conducted using gradient hydrophilic interaction liquid chromatography with tandem mass spectrometry (HILIC/MS/MS). Gingival-brush samples were obtained from the same human panelists in the clinical study as described in EXAMPLE 14, and were placed into extraction buffer as described in EXAMPLE 15. The supernatants were subject to both HILIC/MS/MS and BCA analysis. For free citrulline and ornithine analysis, the extracts of the Gingival-brush samples were analyzed either directly (50 μl undiluted sample solution) in 50/50 acetonitrile/ultra-pure water with 0.754% formic acid or diluted fivefold. For total citrulline and ornithine analysis, the extracts of the Gingival-brush samples were first hydrolyzed using 6 N HCl (50 μL of extract with 450 μL of 6N HCl), no shaking, and placed on a hot plate at 110° C. for 16 hours. The hydrolyzed samples were then dried down under vacuum at room temperature (Savant speedvac of Lifetechnology, Grand Island, N.Y.) and then reconstituted in 1 ml of dilution solution (50/50 acetonitrile/ultra-pure water with 0.754% formic acid) for analysis. The standards and the samples were analyzed using gradient hydrophilic interaction liquid chromatography with tandem mass spectrometry (HILIC/MS/MS). Analytes and the corresponding ISTDs (stable isotope labeled internal standard) were monitored by electrospray ionization (ESI) in positive mode using the selected-reaction-monitoring schemes shown in TABLE 8. A standard curve was constructed by plotting the signal, defined here as the peak area ratio (peak area analyte/peak area ISTD), for each standard versus the mass of each analyte for the corresponding standard. The mass of each analyte in the calibration standards and Gingival-brush extract samples were then back-calculated using the generated regression equation. The concentration of protein bound citrulline or ornithine was calculated as the result of subtracting the concentration of free citrulline or ornithine from the concentration of total citrulline or ornithine, respectively. The result was reported as the concentration of citrulline or ornithine or the result was standardized by dividing by the amount of citrulline or ornithine by the amount of the total proteins that were found in the extract.

TABLE 8
Multiple Reaction Monitoring (MRM) transitions for analytes and
their corresponding stable isotope labeled internal standards.

| Analytes | MRM | Internal Standards | MRM |
|---|---|---|---|
| Citrulline | 176 → 159 | d₇-Citrulline | 181 → 164 |
| Ornithine | 133 → 70 | d₆-Ornithine | 139 → 76 |

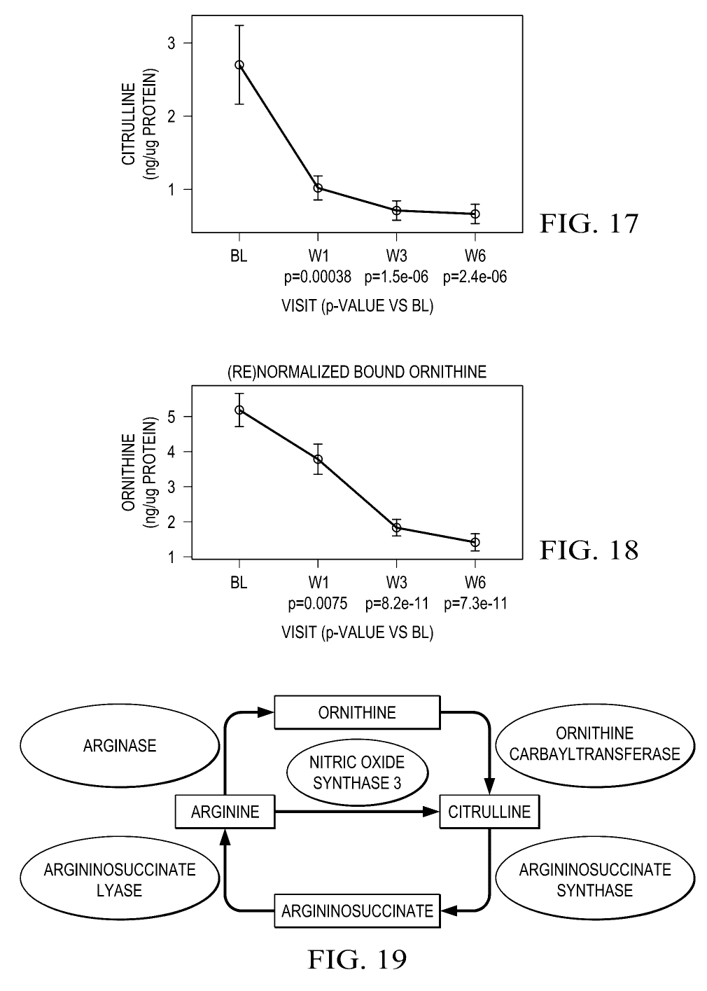

All samples from the panelists of the Test regimen [Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse] were analyzed. As shown in FIG. 17, citrulline levels reduced rapidly in the first week of treatment, and then continued to decline gradually in weeks 3 and 6 of treatment. These results are consistent with clinical observations, where gingival bleeding sites (GBI) and the gingival inflammation (MGI) were reduced over the 6-week treatment period.

### Example 18—Levels of Proteins Containing Ornithine Decreased Over 6 Week Treatment

The same samples as described in EXAMPLE 17 were analyzed using procedures as described in EXAMPLE 17. Gingivitis was treated for 6 weeks. Baseline (BL) represents diseased status. Symptoms of gingivitis were alleviated from week 1 to week 6 treatments. Protein bound ornithine (the difference between total and the free ornithine) was higher in gingivitis as shown in FIG. 18. Protein bound ornithine was reduced gradually as gingivitis was decreased in severity.

### Example 19—Expression of Enzymes in the Ornithine-Citrulline-Arginine Cycle was Changed in Gingival Samples During the 6 Week Treatment

Gingival samples were collected as described in EXAMPLES 15, from the same panelists as in EXAMPLE 15, and were used to examine the expression of genes during 6 weeks of treatments with Test regimen [Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse] and Control regimen [Crest® Cavity Protection (0.243% sodium fluoride) dentifrice and Oral-B® Indicator Soft Manual toothbrush]. After harvesting the samples, the brush was completely immersed in RNAlater solution (1 ml in in a 1.5 ml Eppendorf tube) for keeping RNA from degrading during transport and storage (Qiagen, Valencia, Calif.). The microcentrifuge tubes were vortexed/mixed for 30 seconds, immediately frozen on dry ice, stored and transferred on dry ice to the lab for biomarker analysis. RNA isolation and microarray analysis were performed as described previously in a publication (Offenbacher S, Barros S P, Paquette D W, Winston J L, Biesbrock A R, Thomason R G, Gibb R D, Fulmer A W, Tiesman J P, Juhlin K D, Wang S L, Reichling T D, Chen K S, Ho B. J Periodontol. 2009 December; 80(12):1963-82. doi: 10.1902/jop.2009.080645. Gingival transcriptome patterns during induction and resolution of experimental gingivitis in humans).

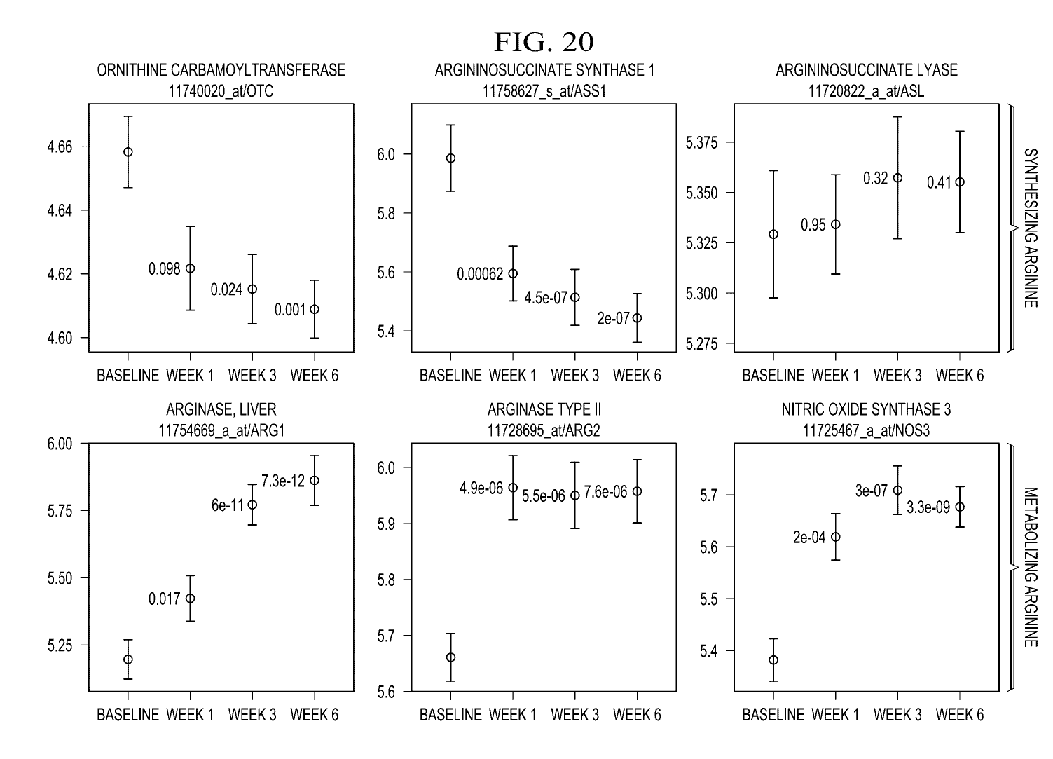

The ornithine-citrulline-arginine cycle consists of four enzymes (FIG. 19). The main feature of the cycle is that three amino acids (arginine, ornithine, and citrulline) can be converted to each other. The first enzyme is ornithine transcarbamoylase, which transfers a carbamoyl group from carbamoyl phosphate to ornithine to generate citrulline. This reaction occurs in the matrix of the mitochondria. Expression of ornithine transcarbamoylase was reduced in the treatment (FIG. 20). The second enzyme is argininosuccinate synthetase. This enzyme uses ATP to activate citrulline by forming a citrullyl-AMP intermediate, which is attacked by the amino group of an aspartate residue to generate argininosuccinate. This and subsequent two reactions occur in the cytosol. Again, expression of argininosuccinate synthetase decreased during the treatment. The third enzyme is argininosuccinate lyase, which catalyzes cleavage of argininosuccinate into fumarate and arginine. The last enzyme is argininase. Argininases cleave arginine to produce urea and ornithine. In a contrast to the decreased expression of ornithine transcarbamoylase and argininosuccinate synthetase genes, argininase I (liver) and II increased (FIG. 20).

Arginine is also a substrate for nitric oxide synthase, which oxidizes arginine to produce citrulline and nitric oxide. Expression of nitric oxide synthase gene increased too (FIG. 20).

### Example 20—Citrulline Increased in Gingival Samples in Experiment Gingivitis

Experimental gingivitis: Another clinical study was carried out to determine whether citrulline is increased in experimentally induced gingivitis in healthy human panelists. This was a case-control study enrolling 60 panelists. The study population included two groups as follows: Group 1 or high bleeders group, thirty (30) panelists with at least 20 bleeding sites, where bleeding is a GBI site score of 1 or 2 at baseline. Group 2 or low bleeders group, thirty (30) panelists with 2 or less bleeding sites, where bleeding is a GBI site score of 1 or 2.

The study consisted of two Phases: Health/Rigorous Hygiene Phase with dental prophylaxis, polishing and rigorous oral hygiene; and Induced Gingivitis Phase without oral hygiene. At the Screening visit, panelists underwent an oral soft tissue assessment and had a gingivitis evaluation (Modified Gingival Index (MGI) and Gingival Bleeding Index (GBI). At Visit 2 qualifying panelists received an oral soft tissue exam followed by a gingivitis evaluation and gingival plaques and gum swabs were collected for the qPCR, protein and RNA host biomarker analysis. Following that, all panelists received dental prophylaxis and entered the Health/Rigorous Hygiene Phase, lasting two weeks. After two weeks of rigorous hygiene, panelists entered the Induced Gingivitis Phase, lasting for three weeks. Oral soft tissue exams and gingivitis were re-evaluated and all samples (gum swabs) were collected at Baseline, WK0 and WK2.

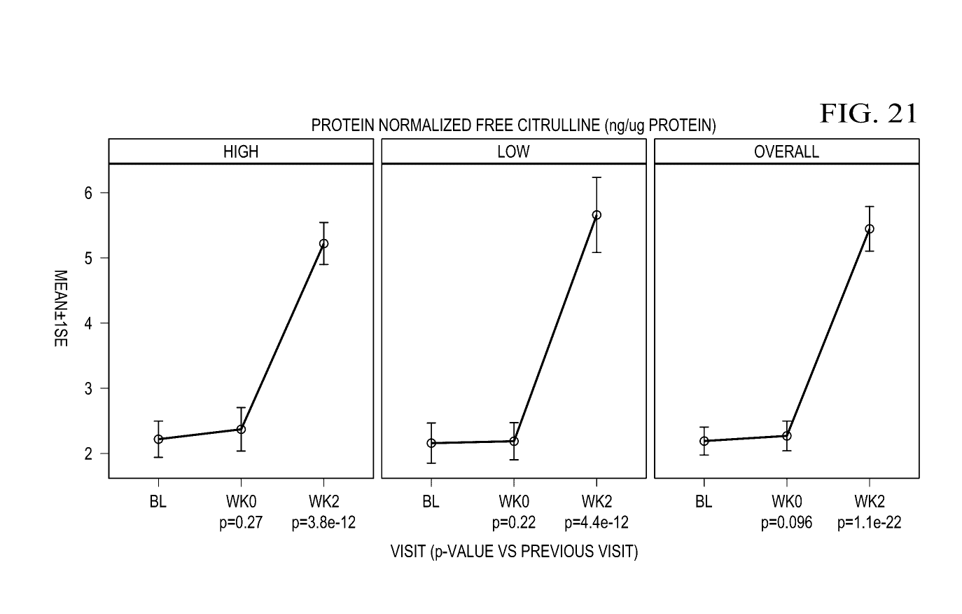

Gingival sample collection—A gum swab was collected from each side of the upper arch using the procedures as described in EXAMPLE 15. Gum swabs were collected close to the gum line from the buccal sites only (preferably from four adjacent teeth—preferably from premolar and molar areas). Panelists rinsed for 30 seconds with 15 ml of Listerine rinse to clean the surface of sampling area. After the Listerine rinse, panelists rinsed for 30 seconds with 20 ml of water. Following that, selected sites were isolated with a cotton roll and gently dried with an air syringe and two gum swabs were taken with collection brushes/swabs from the gingiva region close to the gumline of the selected teeth. The samples were placed in a pre-labeled (panelist ID, sample ID, visit, and date) 1.5 ml micro-centrifuge tube containing 800 ul DPBS (Dulbecco's phosphate-buffered saline) (Lifetechnologies, Grand Island, N.Y.) with protease inhibitors, including AEBSF (4-(2-Aminoethyl) benzenesulfonyl fluoride hydrochloride) 2 mM, aprotinin 0.3 μM, Bestatin 130 μM, EDTA (Ethylenediaminetetraacetic acid) 1 mM, E-64 1 μM, and leupeptin 1 μM. The vials were vortexed/mixed for 30 seconds, immediately frozen on dry ice, stored and transferred on dry ice to the lab for biomarker analysis. Samples from three visits were analyzed using the procedures described in EXAMPLE 17, and shown in FIG. 21. Those three visits were baseline, Week 0, (right after the Health/Rigorous Hygiene Phase and before the induced gingivitis phase) and week 2 (at the end of Induced Gingivitis Phase). Free citrulline levels were low in both the high and low bleeders groups at the baseline and week 0, but rose quickly in the induced gingivitis in both groups at week 2.

### Example 21—Levels of Proteins Containing Citrulline Decreased in Experimentally Induced Gingivitis

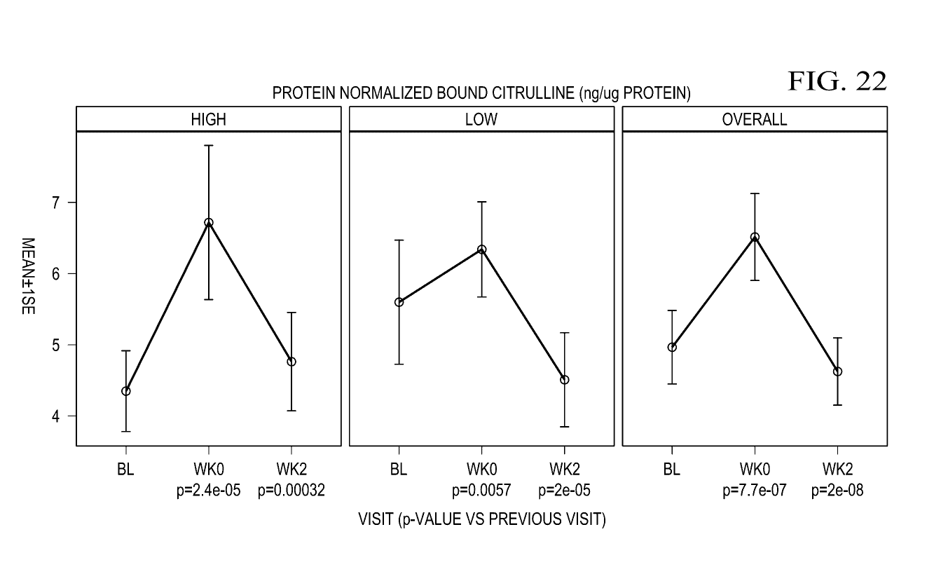

The same procedures were used as described in EXAMPLE 17. The samples were the same as described in EXAMPLE 20. Protein bound citrulline was lower at the baseline than that at week 0 in both high and low bleeders groups as shown in FIG. 22 in gingival tissue. It was low in experimental gingivitis in both groups at week 2.

### Example 22—Levels of Proteins Containing Ornithine Increased in Gingival Samples in Experiment Gingivitis

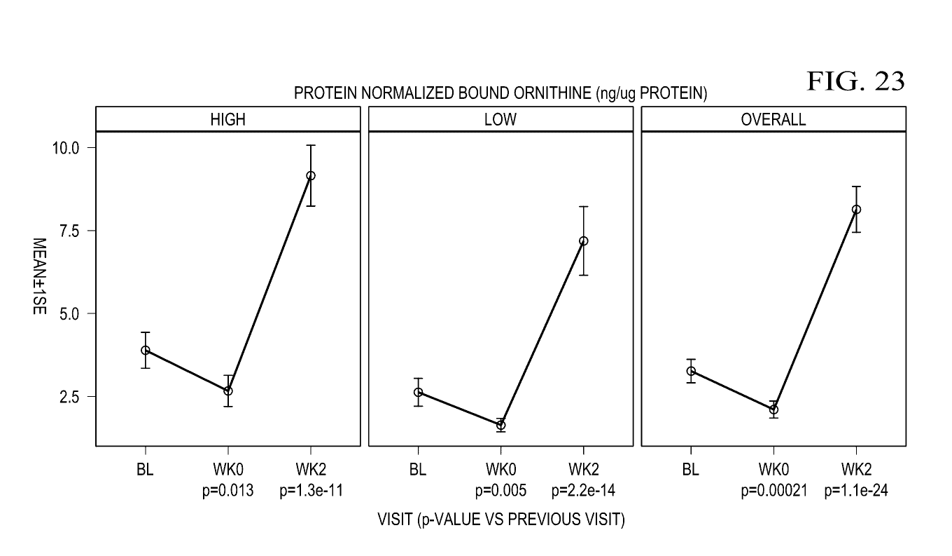

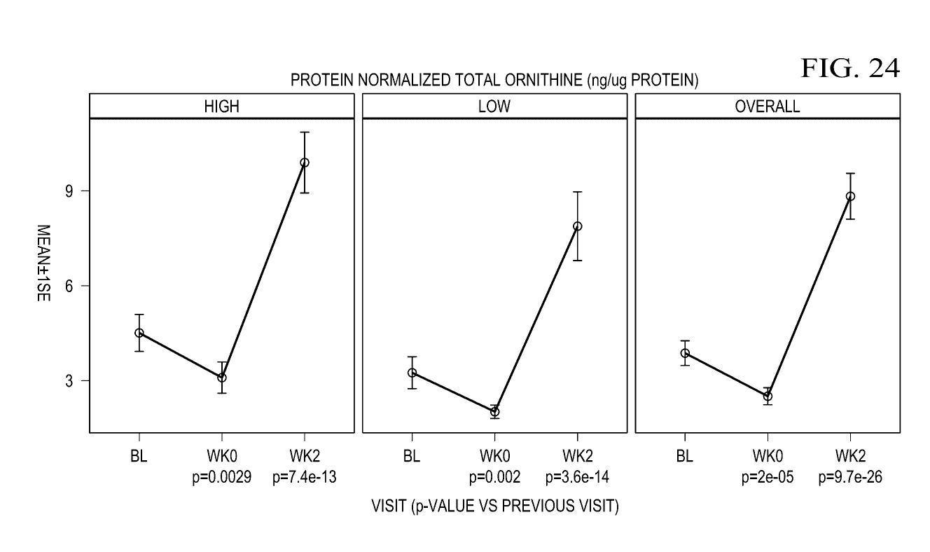

The same clinical samples from experimental gingivitis (EXAMPLE 20) were analyzed using the procedures described in EXAMPLE 17. The bound ornithine was the lowest at week 0 (FIG. 23) in both groups. Its levels at the baseline were higher than those at week 0. The bound ornithine reached peaks when gingivitis was induced in both groups at week 2. Also it is worth noting the total ornithine (Free and protein bound ornithine) was increased in the induced gingivitis (FIG. 24) in both groups.

### Example 23—Levels of Proteins Containing Arginine Decreased in Gingival Samples in Experimentally Induced Gingivitis

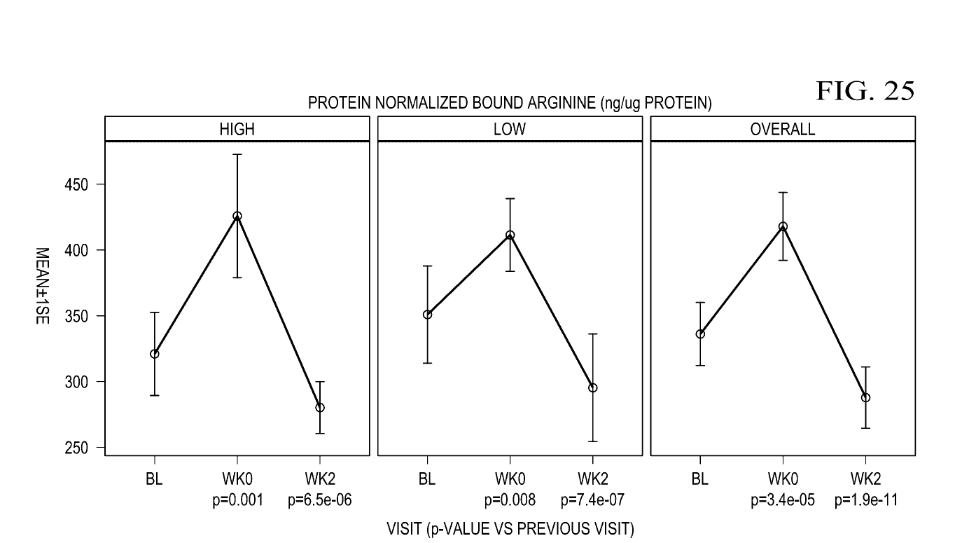

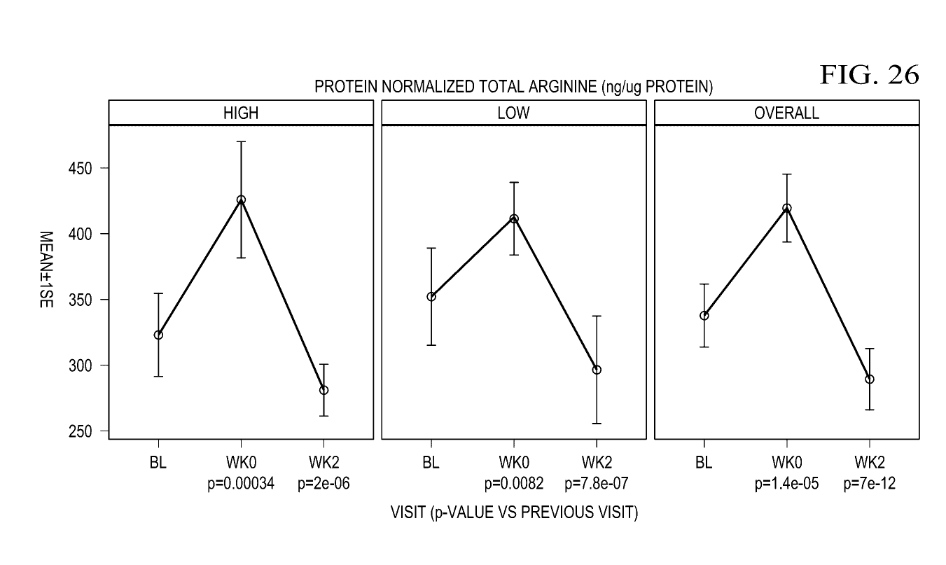

The same procedures were used as described in EXAMPLE 17. The samples were the same as described in EXAMPLE 20. The protein bound arginine was the lowest in induced gingivitis (FIG. 25) in both groups. Its levels were higher in WK0 than at Baseline in both groups. The total arginine in the gingival brush samples displayed the same patterns as the protein bound one (FIG. 26).

### Example 24—Citrulline Inhibited LPS Stimulated Production of Proinflammatory Cytokine in Human THP-1 Cells

Citrulline was purchased from Sigma-Aldrich (St. Louis, Mo.). THP1-Dual™ cells were purchased from Invivogen (San Diego, Calif.). Cells were cultured following the manufacturer's instruction, as described in EXAMPLE 11. For treatment, 0.3 mM to 9 mM of citrulline were first added to the culture medium. Then, 300 ng/ml of P. gingivalis LPS 1690 were added 60 minutes later. After 24 hours of treatment, media was collected and analyzed for cytokine production using 9-plex kit (Meso Scale Diagnostics Rockville, Md.).

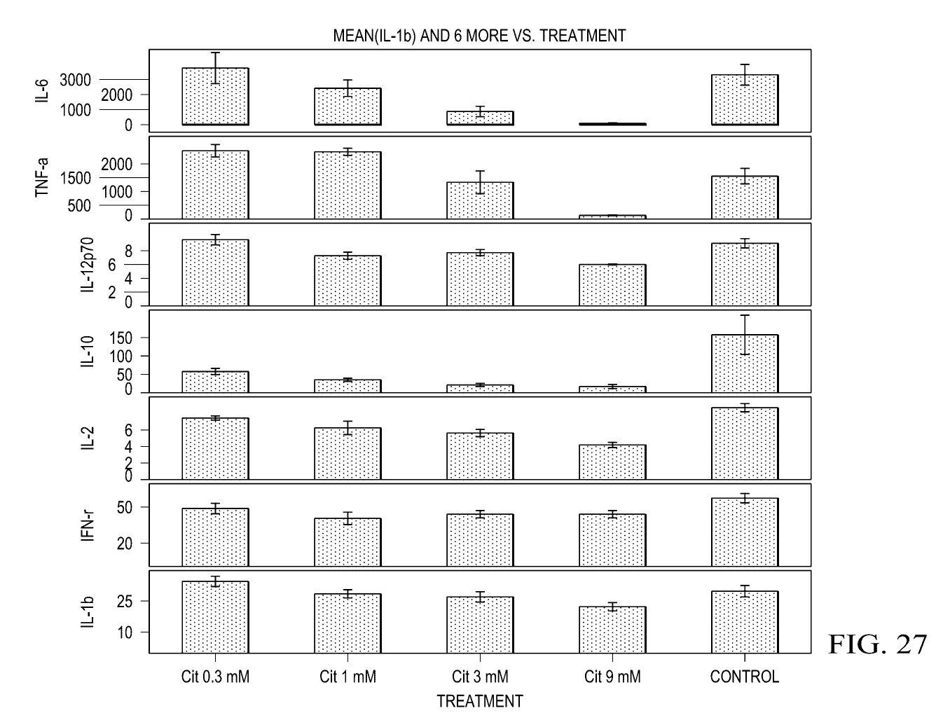

P. gingivalis LPS 1690 stimulated cytokine production, as shown in FIG. 27. Citrulline inhibited P. gingivalis LPS 1690 effects on proinflammatory cytokine production in a dose-dependent manner. Those cytokines include IL-6, TNF-α, IL-12p70, IL-10, IL-2, IFN-r and IL-1β.

### Example 25—Binding of BODIPY-TR-Cadaverine to Bacteria and their Products

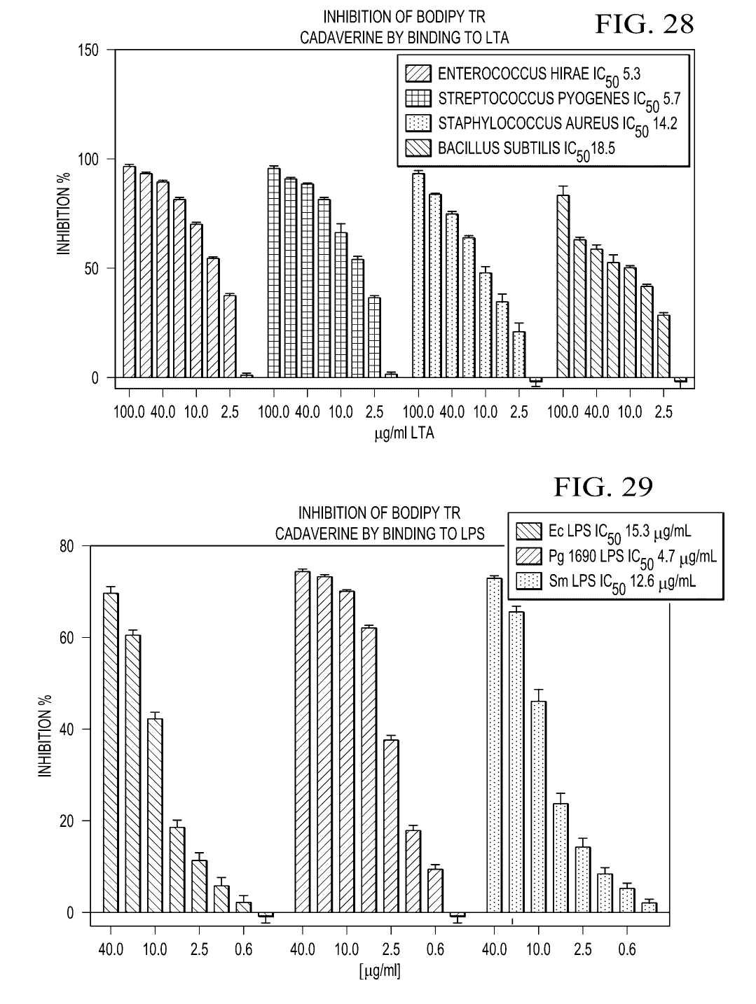

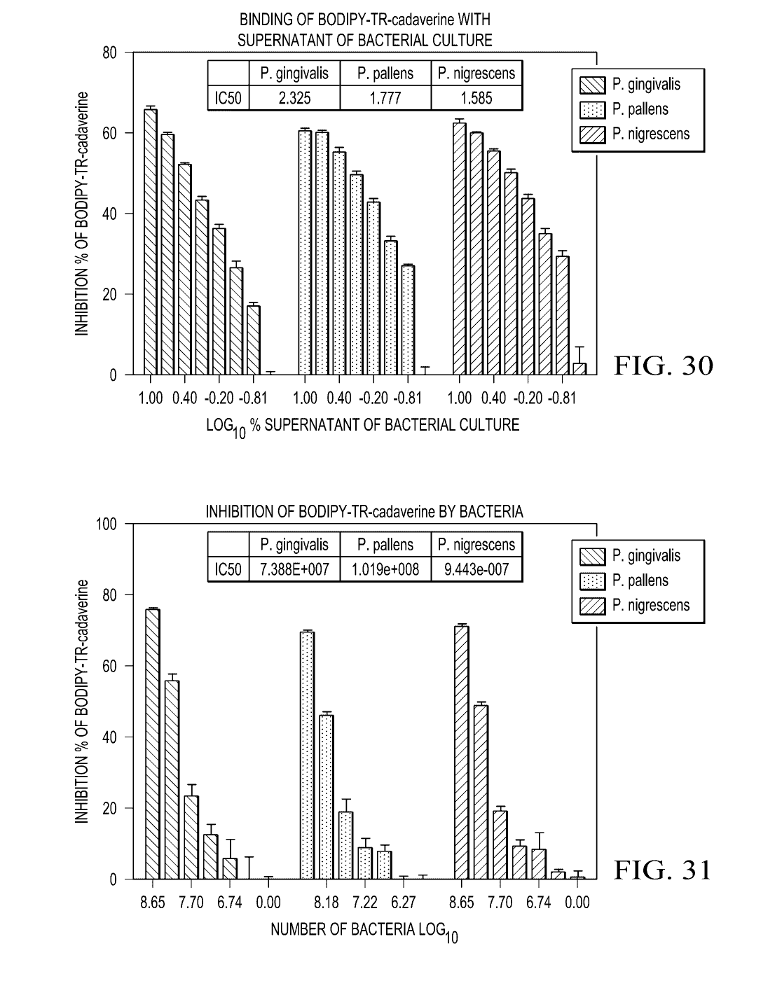

Bacteria and their products can activate TLR2 and TLR4 reporter genes as described in EXAMPLES 5 and 8. Here, experiments were carried out to determine whether different bacteria and their products have different IC50 in reducing fluorescent intensity of BODIPY-TR-cadaverine, hereinafter referred to as BC, in a procedure as described in EXAMPLE 4. Briefly, high throughput screen utilizes the fluorescent dye BODIPY-TR-cadaverine 5-(((4-(4,4-difluoro-5-(2-thienyl)-4-bora-3a,4a-diaza-s-indacene-3-yl)phenoxy)acetyl)amino)pentylamine hydrochloride (BC fluorescent dye), purchased from Life Technologies (Carlsbad, Calif.), as described previously by Wood, Miller and David (2004) (Comb Chem High Throughput Screen 2004 May; 7(3):239-49. Anti-endotoxin agents. 1. Development of a fluorescent probe displacement method optimized for the rapid identification of lipopolysaccharide-binding agents.). The experiment was carried out at room temperature. E. coli LPS (15 μg/ml) was mixed in a well of a 96-well solid black flat bottom plate (Corning Incorporated, Corning, N.Y.) with SnF₂ and cetylpyridinium chloride in 30 μl of 50 mM Tris buffer at pH 7.4 for 10 min, and then 20 μl of 60 μM BC fluorescent dye was added right before fluorescence measurement at 21° C., or room temperature. Fluorescent measurements were performed in a SpectraMax M3 automated 96 well plate reader (Molecular Device, Sunnyvale, Calif.). The excitation wavelength was 580 nM and the fluorescence emission was measured at 620 nM. The half maximal inhibitory concentration (IC₅₀) is a measure of the effectiveness of bacterial products inhibiting BC emitting fluorescence. It indicates how much of a bacterial material is needed to inhibit BC fluorescence by half using GraphPad Prism software (GraphPad Software, La Jolla, Calif.). As shown in FIGS. 28 and 29, each bacterial product decreases the fluorescent intensity of BC in a dose-dependent manner. Similarly, the whole bacterial cells and the supernatant of bacterial growth also reduced the fluorescence of BC in a dose-dependent fashion (FIGS. 30 and 31).

### Example 26—Chemistry Targeting Both LPS and LTA: Binding of Stannous Fluoride to E. coli LPS Using MALDI-TOF Mass Spectrometry

Stannous fluoride is a leading anti-gingivitis technology in P&G toothpaste products. Tests were conducted to understand whether stannous fluoride could bind LPS thus neutralizing LPS proinflammatory activities.

Analysis of E. coli LPS and Stannous Fluoride LPS Binding Using MALDI-TOF Mass Spectrometry: Matrix assisted laser desorption ionization (MALDI) is a soft ionization technique used in mass spectrometry. MALDI mass spectrometry can be used for the analysis of biomolecules such as peptides, proteins, polysaccharides and large organic molecules, such as polymers. In MALDI, the analyte is first co-crystallized with a UV absorbing matrix, such as alpha-cyano-4-hydroxycinnamic acid (CHCA), then subjected to pulse YAG laser radiation. This causes the desorption of the analyte/matrix crystals and produces ions which are transmitted into a mass analyzer for detection. In MALDI-TOF, a time-of-flight mass analyzer is used. MALDI-TOF data can be acquired in MS mode to generate molecular weight information and in MS/MS mode for structure information. Typical MALDI mass spectrum acquisition takes less than a minute so it can be used for fast screening of molecular species in samples of interest. In this study, E. coli LPS molecular weight profiles and its interaction with SnF₂ were investigated. An equal volume of the E. coli LPS solutions with or without SnF₂ was mixed with the 10 μl MALDI matrix CHCA (10 mg/ml in 80% acetonitrile/20% water). 0.7 ul of the sample solution was spotted on a MALDI plate, air-dried, and analyzed in negative ion mode using a MALDI-TOF/TOF 4800 plus system (AB-Sciex, Framingham, Mass., USA). Typical data acquisition uses mass scan range 500-3000 Da and a laser power of 4500. Data was collected in an automated fashion using random sampling over the sample spot with 250 shots per subspectrum and a total of 2500 shots per spectrum.

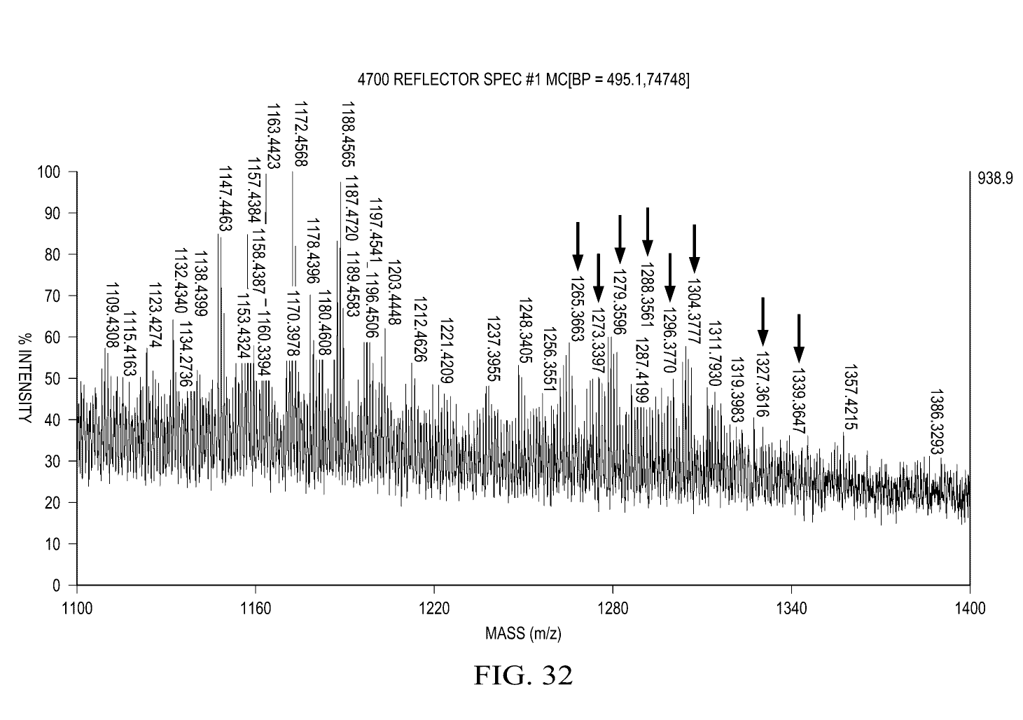

The data shown in FIG. 32 showed E. coli LPS is heterogeneous with atomic masses range from 1130 to 1220. This observation was consistent with literature reports. Some of the LPS did bind to one molecule of stannum, as marked with an arrow. Similarly, stannum is the element with the largest number of stable isotopes having atomic masses ranging from 112 to 124. The most abundant ones are 120Sn, 118Sn, and 116Sn, while the least abundant one is 115Sn. Three different isotopes were identified in the sample, with 116Sn most abundant.

### Example 27—Chemistry Targeting Both LPS and LTA: Identification of Materials that Displace Binding of LPS from Fluorescent Dye BODIPY-TR-Cadaverine

Virulence factors are toxic to host cells and tissue, causing damages, injuries, death or serious debilitation. A detoxifying agent is able to target, bind to and neutralize bacterial virulence factors, such as LPS and LTA. In certain embodiments a detoxifying agent may be present in an amount of from about 0.001% to about 2.0%, by weight of the oral care composition. To develop such LPS and LTA sequestration technologies, or detoxifying agent, a high throughput screening, as described briefly in EXAMPLE 4, was employed to identify molecules that disrupt the activation of a toll-like receptor by LPS and LTA, and other virulence factors. The high throughput screen utilizes the fluorescent dye BODIPY-TR-cadaverine 5-(((4-(4,4-difluoro-5-(2-thienyl)-4-bora-3a,4a-diaza-s-indacene-3-yl)phenoxy)acetyl)amino)pentylamine hydrochloride (BC fluorescent dye), purchased from Life Technologies (Carlsbad, Calif.), as described previously by Wood, Miller and David (2004) (Comb Chem High Throughput Screen. 2004 May; 7(3):239-49. Anti-endotoxin agents. 1. Development of a fluorescent probe displacement method optimized for the rapid identification of lipopolysaccharide-binding agents.) E. coli LPS (15 μg/ml) was mixed with test materials believed to be safe to humans cosmetically in 30 μl of 50 mM Tris buffer at pH 7.4 for 10 min, and then 20 μl of 60 μM BC fluorescent dye was added right before fluorescence measurement at 21° C. Fluorescence measurements were performed on automated 96 well plate reader SpectraMax M3 (Molecular Device, Sunnyvale, Calif.). The excitation wavelength was 580 nM and the fluorescence emission was measured at 620 nM.

To identify technologies that sequestrate LPS from binding to BODIPY TR cadaverine, internal chemical libraries were screened. Compounds that displaced LPS binding by 15% were considered as hits; with the initial hits tested again.

As shown in TABLE 9, certain materials showed strong displacement activities.

TABLE 9
Compounds replacing LPS from binding to BODIPY TR cadaverine.

| | LPS displacement % | | | |
|---|---|---|---|---|
| Chemicals | Reading 1 | Reading 2 | Reading 3 | Mean |
| Steapyrium chloride | ~100 | 100 | ~100 | ~100 |
| N-Laurylpyridinium chloride | 66 | 56 | 59 | 60 |
| N,N-Dimethyldodecylamine | 36 | 18 | 13 | 22 |
| Myristyl-.gamma.-picolinium chloride | ~100 | ~100 | ~100 | ~100 |
| Diethylaminoethyl stearamide | 77 | 62 | 58 | 66 |
| Laurylisoquinolinium bromide | ~100 | 39 | 66 | 69 |
| N-Myristoylglycine | ~100 | 39 | 74 | 76 |
| Stearylamidopropyldimethylamine | 85 | 71 | 74 | 77 |
| Diallyldimethylammonium chloride | 40 | 38 | 28 | 35 |
| 3-Dodecanamidopropyldimethylamine | 87 | 62 | 87 | 79 |
| dl-lactic acid hemizinc salt | 15 | | 24 | 19 |
| 1,3-Diphenylacetone (1,3 diphenyl-2-propanone) | 16 | | 5 | 11 |
| alpha-Amylcinnamaldehyde | 17 | | 13 | 15 |
| cis-Oleic acid | 54 | | 9 | 31 |
| Isopentyl benzoate | 22 | | 13 | 18 |
| Undecylenic aldehyde | 29 | | 15 | 22 |
| iron(iii) chloride hexahydrate | ~100 | 100 | ~100 | ~100 |
| 3-(4-Isopropylphenyl)-2-methylpropionaldehyde | 21 | 29 | 65 | 39 |
| Copper(I) iodide | 23 | 12 | 20 | 18 |
| Methyl linoleate | ~100 | 53 | ~100 | 88 |

### Example 28—Chemistry Targeting Both LPS and LTA: Identification of LPS Binding Materials Using Nearest Neighbor Similarity Searching

Compounds listed in TABLE 9 were used as starting materials for nearest neighbor similarity searching versus with test materials believed to be safe to humans cosmetically. The software used for nearest neighbor similarity searching was Pipeline Pilot software (version 8.5.0.2 from BIOVIA, San Diego, Calif.), which included chemoinformatics components.

Nearest neighbor similarity searching involves 4 basic steps—Structure generation; Fingerprint generation from the structure; Calculation of the distance between two fingerprints; and Application of a cutoff to filter results.
1. Structure generation—structures for the 6 lead compounds, as shown below (Chlorhexidine, Alexidine, Yan-673, DS-176, Cetylpyridinium chloride, DS-347), were drawn manually using ChemBioDraw Ultra v.14 and converted into SMILES strings (1D representation of structure). The target ListNet database already contained structures as SMILES strings. All lead and target structures were imported into Pipeline Pilot and the “Molecule from SMILES” component was used to convert all SMILES to 2D molecular structures.

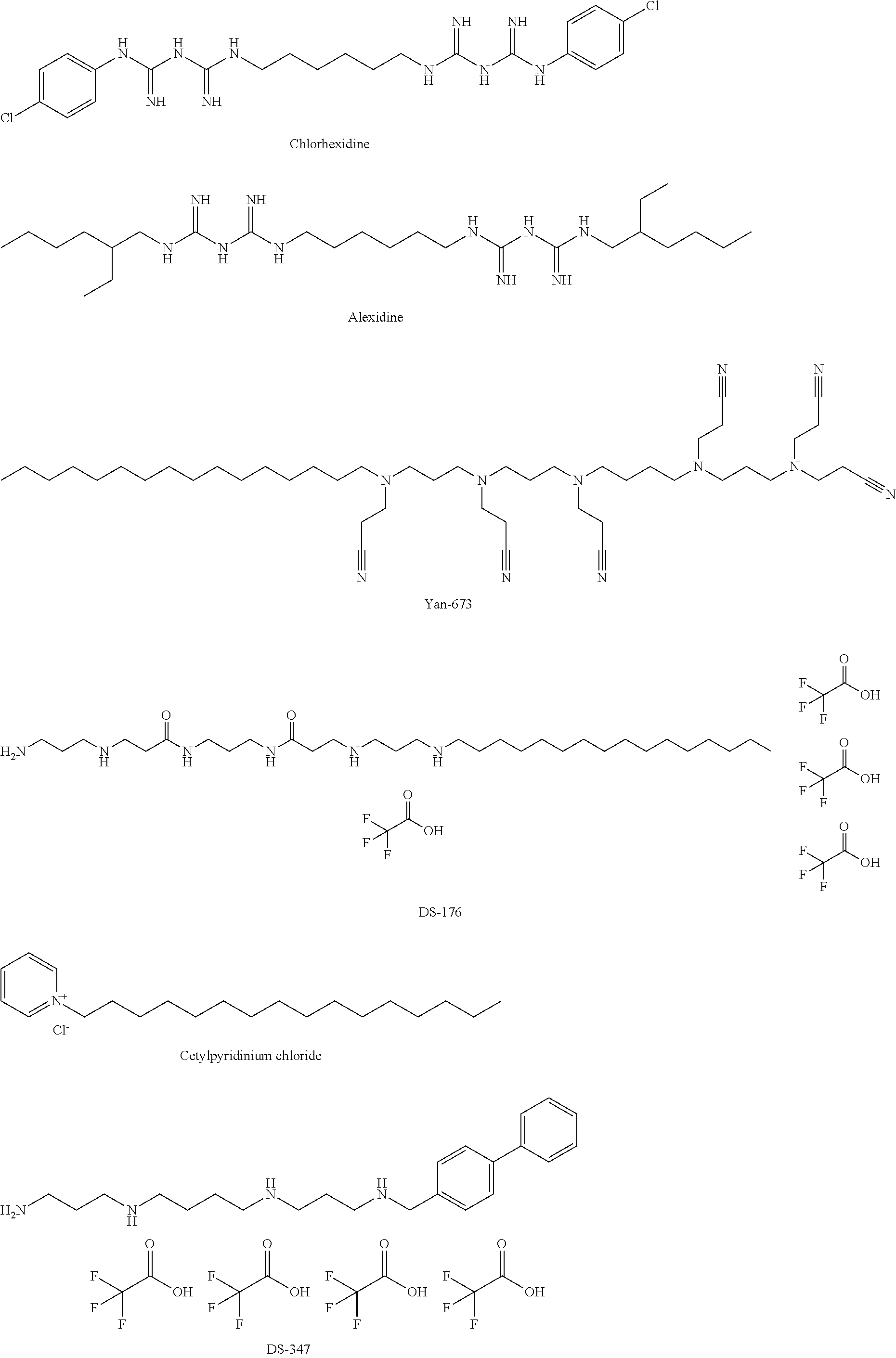

2. Fingerprint generation—The FCFP_6 fingerprint method was used to generate all molecular fingerprints.
   Functional Class Extended-Connectivity FingerPrint is a method for generating atom feature codes for all heavy (non-hydrogen) atoms in a molecule. It applies the following rapid functional-role codes to each atom: Hydrogen-bond acceptor, Hydrogen-bond donor, Positively ionized or positively ionizable, Negatively ionized or negatively ionizable, Aromatic and Halogen. The number after the underscore indicates the maximum distance for extending the connectivity. In this case, FCFP_6 indicates that the connectivity is extended up to 6 bonds in diameter.
   FCFPs were developed by Accelrys Inc. and incorporated in their Pipeline Pilot software (“Extended-Connectivity Fingerprints”, D. Rogers and M. Hahn, J. Chem. Inf Model., 2010, 50 (5), pp 742-754). The protocols were run as described in the Accelrys' Pipeline Pilot user guide (Accelrys Software Inc., Chemistry Collection: Basic Chemistry User Guide, Pipeline Pilot, San Diego: Accelrys Software Inc., 2011).
3. Calculation of distance—The Tanimoto similarity coefficient was used to calculate similarity values between each database molecule and the lead molecules encoded by the FCFP_6 molecular fingerprints described above using Pileline Pilot's “Find similar molecules fingerprint” component.
   The Tanimoto similarity coefficient is defined by the expression:

$$Tanimoto = \frac{SA}{SA + SB + SC}$$

where:
SA=Number of bits defined in both the target and the reference
SB=Number of bits defined in the target but not the reference
SC=Number of bits defined in the reference but not the target
The Tanimoto similarity ranges from zero (there are no common ‘bits’ between the reference and the target molecules) to one (the reference and the target molecules have exactly the same ‘bits’)
4. Cutoff and filtering—A cutoff distance of 0.3 was applied to filter the results. This resulted in 76 materials being selected out of 4456. The 76 compounds were manually/visually analyzed to remove self-matches, multiple salt forms and duplicates and narrowed to 29 top candidates which were recommended for lab testing. 10 of the 29 materials were confirmed hits in an LPS displacement assay.

Using nearest neighbor similarity searching, we found a list of compounds (TABLE 10) that share structural similarities to LPS binders listed in TABLE 9.

TABLE 10
Materials of sharing structural similarities to LPS binders.

| CAS | Smiles | Common Name |
|---|---|---|
| 14246-55-0 | CCCCCCCCCCCCCC(═O)NCC(═O)O | N-Myristoyl-glycine |
| 14246-53-8 | CCCCCCCC(═O)NCC(═O)O | Caprylylglycine |
| 112-63-0 | CCCCC/C═C\C/C═C\CCCCCCCC(═O)OC | Methyl Linoleate |
| 112-62-9 | CCCCCCCC/C═C\CCCCCCCC(═O)OC | Methyl Oleate |
| 1120-34-9 | CCCCCCCC/C═C\CCCCCCCCCCCC(═O)OC | Methyl cis-13-Docosenoate |
| 301-00-8 | CC/C═C\C/C═C\C/C═C\CCCCCCCC(═O)OC | Methyl linolenate |
| 22882-95-7 | CCCCC/C═C\C/C═C\CCCCCCCC(═O)OC(C)C | isopropyl linoleate |
| 544-35-4 | CCCCC/C═C\C/C═C\CCCCCCCC(═O)OCC | Ethyl Linoleate |
| 17673-59-5 | CCCCCCCC/C═C\CCCCCCCCOC(═O)CCCCCCC/C═C\C/C═C\CCCCC | oleyl linoleate |
| 106-73-0 | CCCCCCC(═O)OC | methyl heptanoate |
| 7132-64-1 | CCCCCCCCCCCCCCC(═O)OC | Methyl pentadecanoate |
| 1731-88-0 | CCCCCCCCCCCCC(═O)OC | Methyl tridecanoate |
| 124-10-7 | CCCCCCCCCCCCCC(═O)OC | Methyl Myristate |
| 1731-84-6 | CCCCCCCCC(═O)OC | methyl nonanoate |
| 1808-26-0 | CCCCC/C═C\C/C═C\C/C═C\C/C═C\CCCC(═O)OCC | Arachidonic Acid ethyl ester |
| 111-82-0 | CCCCCCCCCCCC(═O)OC | methyl laurate |
| 111-11-5 | CCCCCCCC(═O)OC | methyl octanoate |
| 112-39-0 | CCCCCCCCCCCCCCCC(═O)OC | Palmitic Acid methyl ester |
| 110-42-9 | CCCCCCCCCC(═O)OC | Methyl Decanoate |
| 112-11-8 | CCCCCCCC/C═C\CCCCCCCC(═O)OC(C)C | isopropyl oleate |
| 111-62-6 | CCCCCCCC/C═C\CCCCCCCC(═O)OCC | Oleic Acid ethyl ester |
| 56219-10-4 | CCCCCC/C═C\CCCCCCCC(═O)OCC | Palmitoleic Acid ethyl ester |
| 1191-41-9 | CC/C═C\C/C═C\C/C═C\CCCCCCCC(═O)OCC | ethyl linolenate |
| 141-24-2 | CCCCCC[C@H](C/C═C\CCCCCCCC(═O)OC)O | Methyl Ricinoleate |
| 106-70-7 | CCCCCC(═O)OC | methyl hexanoate |
| 2432-77-1 | CCCCCC(═O)SC | S-Methyl thiohexanoate |
| 140-03-4 | CCCCCC[C@H](C/C═C\CCCCCCCC(═O)OC)OC(═O)C | Methyl O-Acetylricinoleate |
| 537-40-6 | CCCCC/C═C\C/C═C\CCCCCCCC(═O)OCC(OC(═O)CCCCCCC/C═C\C/C═C\CCCCC)COC(═O)CCCCCCC/C═C\C/C═C\CCCCC | Glyceryl trilinoleate |
| 111-57-9 | CCCCCCCCCCCCCCCCCC(═O)NCCO | Stearoyl Ethanolamide |
| 142-78-9 | CCCCCCCCCCCC(═O)NCCO | Lauramide MEA |
| 13481-87-3 | CCCCC/C═C/CC(═O)OC | methyl 3-nonenoate |
| 544-31-0 | CCCCCCCCCCCCCCCC(═O)NCCO | Palmitoyl Ethanolamide |
| 2277-28-3 | CCCCC/C═C\C/C═C\CCCCCCCC(═O)OCC(CO)O | (S)-glyceryl linoleate |
| 142-77-8 | CCCCCCCC/C═C\CCCCCCCC(═O)OCCCC | Butyl oleate |
| 111-81-9 | COC(═O)CCCCCCCCC═C | methyl 10-undecenoate |
| 3687-46-5 | CCCCCCCCCCOC(═O)CCCCCCC/C═C\CCCCCCCC | Decyl Oleate |
| 22393-86-8 | CCCCCCCCCCCCCCCCOC(═O)CCCCCCC/C═C/CCCCCCCC | Palmityl Oleate |
| 36078-10-1 | CCCCCCCCCCCCOC(═O)CCCCCCC/C═C/CCCCCCCC | Dodecyl Oleate |
| 22393-93-7 | CCCCCCCCCCCCCC(═O)OCCCCCCCC/C═C\CCCCCCCC | Oleyl Myristate |
| 2441-41-0 | CCCCCCCCCCCCCCCC(═O)NCC(═O)[O−] | N-Palmitoyl Glycine |
| 56255-31-3 | CCCCCCCCCCCCCCCC(═O)N[C@@H](C)C(═O)O | N-Hexadecanoyl-alanine |
| 21394-64-9 | CCCCCCCCCCCCCCCC(═O)NC(CO)C(═O)O | N-Palmitoyl Serine |
| 4500-01-0 | CCCCCCCC/C═C\CCCCCCCC(═O)OCCO | Glycol Oleate |
| 52315-75-0 | CCCCCCCCCCCC(═O)NCCCC[C@@H](C(═O)O)N | Lauroyl Lysine |
| 111-79-5 | CCCCCC/C═C/C(═O)OC | Methyl trans-2-nonenoate |
| 30606-27-0 | CCCCC/C═C\C/C═C\CCCCCCCC(═O)OCC(CO)OC(═O)CCCCCCC/C═C\C/C═C\CCCCC | Glyceryl Dilinoleate |
| 106-79-6 | COC(═O)CCCCCCCCC(═O)OC | Dimethyl Sebacate |
| 1472-87-3 | COC(═O)CCCCCCCCCCCC(═O)OC | Dimethyl Brassylate |
| 105-62-4 | CCCCCCCC/C═C\CCCCCCCC(═O)OCC(C)OC(═O)CCCCCCC/C═C\CCCCCCCC | propylene glycol dioleate |
| 122-32-7 | CCCCCCCC/C═C\CCCCCCCC(═O)OCC(OC(═O)CCCCCCC/C═C\CCCCCCCC)COC(═O)CCCCCCC/C═C\CCCCCCCC | Triolein |
| 2752-99-0 | CCCCCCCC/C═C\CCCCCCCCCCCC(═O)OCC(OC(═O)CCCCCCCCCCC/C═C\CCCCCCCC)COC(═O)CCCCCCCCCCC/C═C\CCCCCCCC | Trierucin |
| 20246-55-3 | CCCCCC/C═C\CCCCCCCC(═O)OCC(OC(═O)CCCCCCC/C═C\CCCCCC)COC(═O)CCCCCCC/C═C\CCCCCC | Glyceryl Tripalmitoleate |
| 624-24-8 | CCCCC(═O)OC | Methyl Valerate |
| 7367-81-9 | CCCCC/C═C/C(═O)OC | methyl (E)-2-octenoate |
| 60-33-3 | CCCCC/C═C\C/C═C\CCCCCCCC(═O)O | Linoleic acid |
| 3999-01-7 | CCCCC/C═C\C/C═C\CCCCCCCC(═O)N | 9,12-Octadecadienamide |
| 76649-16-6 | CCCCC/C═C/CCC(═O)OCC | ethyl (E)-4-decenoate |
| 25151-96-6 | CCCCCCCC/C═C\CCCCCCCC(═O)OCC(COC(═O)CCCCCCC/C═C\CCCCCCCC)(CO)CO | pentaerythrityl dioleate |
| 111-03-5 | CCCCCCCC/C═C\CCCCCCCC(═O)OCC(CO)O | 1-glyceryl oleate |
| 10332-32-8 | CCCCCCCC/C═C\CCCCCCCC(═O)OCC(CO)(CO)CO | 3-hydroxy-2,2-bis(hydroxymethyl)propyl oleate |
| 56863-02-6 | CCCCC/C═C\C/C═C\CCCCCCCC(═O)N(CCO)CCO | Linoleamide DEA |
| 18465-99-1 | CC/C═C\C/C═C\C/C═C\CCCCCCCC(═O)OCC(CO)O | glyceryl linolenate |
| 627-93-0 | COC(═O)CCCCC(═O)OC | Dimethyl adipate |
| 141-23-1 | CCCCCCC(CCCCCCCCCCC(═O)OC)O | Methyl 12-Hydroxy Stearate |
| 3414-89-9 | CCCCC/C═C\C/C═C\CCCCCCCC(═O)[O−]•[K+] | potassium linoleate |
| 16889-14-8 | CCCCCCCCCCCCCCCCCC(═O)NCCN(CC)CC | stearamidoethyl diethylamine |
| 111-58-0 | CCCCCCCC/C═C\CCCCCCCC(═O)NCCO | Oleoyl Ethanolamide |
| 506-32-1 | CCCCC/C═C\C/C═C\C/C═C\C/C═C\CCCC(═O)O | |
| 506-26-3 | CCCCC/C═C\C/C═C\C/C═C\CCCCC(═O)O | Arachidonic Acid |
| 106-12-7 | CCCCCCCC/C═C\CCCCCCCC(═O)OCCOCCO | diethylene glycol monooleate |
| 38079-66-2 | CCCCCCCCCCCCCCCC(═O)NC(CCC(═O)O)C(═O)O | palmitoyl glutamic acid |
| 7651-02-7 | CCCCCCCCCCCCCCCCCC(═O)NCCCN(C)C | Stearamidopropyl dimethylamine |
| 947-04-6 | C1CCCCCC(═O)NCCCCC1 | 12-Aminododecanolactam |
| 3397-65-7 | CCCCCCCCCCCC(═O)N[C@@H](CCC(═O)O)C(═O)O | lauroyl glutamic acid |
| 111-05-7 | CCCCCCCC/C═C\CCCCCCCC(═O)NCC(C)O | N-(2-hydroxypropyl)oleamide |
| 3179-80-4 | CCCCCCCCCCCC(═O)NCCCN(C)C | lauramidopropyl dimethylamine |
| 5422-34-4 | OCCNC(═O)C(C)O | Lactamide MEA |
| 142-26-7 | CC(═O)NCCO | Acetamide MEA |
| 68140-00-1 | Mixture of structures | Cocamide MEA |
| 94109-05-4 | O═C(NCCO)CCCCCCCCCCCCCCCCCCCCC | Behenamide MEA |
| 68171-52-8 | O═C(NCCO)CCCCCCC\C═C/C\C═C/CCCCC | Linoleamide MEA |
| 242132-61-2 | NC(═O) CCCCCCCC(═O) N | Azelamide MEA |
| 106-15-0 | O═C(NCCO)CCCCCCCCCCC(O)CCCCCC | Hydroxystearamide MEA |
| 54536-43-5 | O═C(N(CCO)CCO)CCCCCCCCCCCCCCC(C)C | Isostearamide MEA |
| 996-97-4 | O═C(N(CC)CC)CCCCCCC | Octamide MEA |
| 7757-97-3 | CC(C)(CO)[C@H](C(═O)NCCC(═O)N)O | Pantothenamide MEA |
| 106-16-1 | O═C(NCCO)CCCCCCC/C═C/CC(O)CCCCCC | Ricinoleamide MEA |
| 25377-63-3 | O═C(NCCO)CCCCCCCC\C═C | Undecylenamide MEA |

The outlined methodology of using: (1) the LAL kit to quantify total LPS present and to detect technologies that inhibit the LAL assay (EXAMPLES 1-3); (2) using the toll like receptor assays to determine potency of purified virulence and oral dental plaques, and to measure the efficacy of technologies that neutralize toxicity of virulence factors (EXAMPLES 4-12); and (3) the high throughput screening and nearest neighbor similarity searching to identify new LPS and LTA mitigation technology (EXAMPLES 25-30); surprisingly lead to molecules that reduced the virulence of subgingival plaque, which led to a reduction in toxicity of the plaque. This reduction in toxicity was a result of the identified chemistry detoxifying the subgingival environment, which surprisingly led to better oral health as was manifested in a reduction in gingivitis.

TABLE 11
LPS Inhibition Via Limulus Amebocyte Lysate Assay.

| | LAL % |
|---|---|
| STRUCTURE_NAME | Inhibition |
| Hexaethylene glycol, monodecyl ether | 100.50 |
| Laureth-6 | 100.44 |
| Steapyrium chloride | 100.13 |
| N-Lauryldiethanolamine | 100.05 |
| Quaternium-17 | 100.01 |
| Laureth-9 | 99.92 |
| Bromoenol lactone | 99.69 |
| N-Lauryldiethanolamine | 99.64 |
| Oleyl amine | 99.60 |
| Cetrimonium chloride | 99.51 |
| Cetyltrimethylammonium bromide | 99.45 |
| Dimethylaminopropyllaurylamide | 99.40 |
| Cetylpyridinium chloride | 99.13 |
| (Laurylamidopropyl)dimethyl amine | 99.09 |
| potassium linoleate | 98.92 |
| Dimethyldodecylbenzylammonium chloride | 98.25 |
| Dimethyldodecylbenzylammonium bromide | 98.09 |
| .alpha.-linoleic acid | 97.88 |
| Benzalkonium chloride | 97.50 |
| Didecyldimethylammounium chloride | 97.49 |
| Diethylaminoethyl stearamide | 97.42 |
| Laurylisoquinolinium bromide | 97.41 |
| (R)-Bromoenol lactone | 97.27 |
| Laurylisoquinolinium bromide | 96.94 |
| C-8 Ceramine | 96.71 |
| N-Laurylpyridinium chloride | 96.53 |
| Stearalkonium chloride | 96.47 |
| Myristyl-.gamma.-picolinium chloride | 96.23 |
| TRIDECYL SULFONIC ACID SODIUM SALT | 95.58 |
| Dimethylbenzylcetylammonium chloride | 95.55 |
| Domiphen bromide | 95.54 |
| (S)-Bromoenol lactone | 95.15 |
| Benzethonium chloride | 94.57 |
| Tetradecylbenzyldimethylammonium chloride | 93.78 |
| Dioctyldimethylammonium chloride | 93.30 |
| Abacross Flaxseed Oil | 93.24 |
| Tea Tree Oil Fractionated (Melafresh T96) | 93.16 |
| Methylbenzethonium chloride | 92.98 |
| N-Myristoylglycine | 91.40 |
| DODECYLTRIPHENYLPHOSPHONIUM BROMIDE, 98% | 91.14 |
| fenugreek | 90.00 |
| Sodium hexadecyl sulfate | 89.44 |
| STEARYLDIETHANOLAMINE | 88.09 |
| Sandalwood Oil (West Australian) | 87.78 |
| Asian ginseng | 87.07 |
| Tin tetrachloride | 86.74 |
| Marjoram Sweet Oil | 86.64 |
| Sodium pentadecyl sulfate | 86.61 |
| Propylpyrazole Triol | 86.58 |
| Sandalwood Oil (Australian) | 86.06 |
| Sandalwood Oil | 85.98 |
| N-Palmitoyl Glycine | 85.78 |
| N-Docosanoyl Taurine | 85.15 |
| Stearylamidopropyldimethylamine | 83.77 |
| Eicosapentaenoyl PAF C-16 | 82.66 |
| 5.alpha.-hydroxy-6-keto Cholesterol | 81.72 |
| N-Nervonoyl Taurine | 79.82 |
| FTY720 | 79.08 |
| linoleamide | 75.25 |
| Abacross Pepper Black Oil | 73.67 |
| PAz-PC | 72.82 |
| Steartrimonium chloride | 71.27 |
| 1,2-Didecanoyl PC | 67.90 |
| diglycol monooleate | 67.01 |
| Frankincense Oil | 66.66 |
| C-8 Ceramide | 65.48 |
| D-erythro-Sphingosine C-18 | 63.76 |
| Oleic Acid | 61.04 |
| Methylcarbamyl PAF C-16 | 59.72 |
| Lavender Oil | 58.69 |
| Ferric chloride hexahydrate | 58.13 |
| N,N-Dimethylsphingosine | 58.11 |
| Oleyl Trifluoromethyl Ketone | 57.56 |
| Arisaema Amurense Rhizome | 56.66 |
| Curcumin | 55.88 |
| 1-Stearoyl-2-Arachidonoyl PC | 54.96 |
| .delta.-Tocotrienol | 52.44 |
| Arachidonyl Trifluoromethyl Ketone | 52.11 |
| 1,2-Dioctanoyl-sn-glycerol | 51.18 |
| N-Arachidonoyl Dopamine | 49.13 |
| .alpha.-glyceryl monooloeate | 48.98 |
| aloe Vera | 48.93 |
| POV-PC | 48.72 |
| C-6 Ceramide | 48.05 |
| AM404 | 47.57 |
| 1,2-Didecanoyl-sn-glycerol | 47.40 |
| MK 886 (sodium salt) | 47.19 |
| 9-octadecenoic acid (z)-,2-hydroxyethyl ester | 46.04 |
| Dodecanamide MEA | 44.32 |
| Lavender Eucalyptus blend | 44.25 |
| Abacross Mandarin Red Oil | 43.62 |
| C-8 Ceramide-1-phosphate | 42.66 |
| Hexadecyl Acetyl Glycerol | 41.98 |
| 1-Arachidonoyl Glycerol | 41.75 |
| Hexadecyl Methyl Glycerol | 39.97 |
| Montelukast (sodium salt) | 38.55 |
| .gamma.-Tocotrienol | 38.53 |
| methyl linoleate | 36.90 |
| 1-Oleoyl-2-acetyl-sn-glycerol | 36.17 |
| Celastrol | 35.93 |
| 2-Arachidonyl Glycerol ether | 34.98 |
| (z,z,z)-9,12,15-octadecatrienoic acid ethyl ester | 34.94 |
| Ebselen | 34.70 |
| Oleoylethanolamide | 34.14 |
| Palmityl Trifluoromethyl Ketone | 34.08 |
| Oxirane, polymer with formaldehyde and 4-(1,1,3,3- | 33.80 |
| tetramethylbutyl)phenol | |
| Hexadecanamide MEA | 32.47 |
| ethyl cis-9-hexadecenoate | 31.75 |
| Anandamide (N-arachidonylethanol amide) | 31.31 |
| Olive Oil | 30.72 |
| alpha.-Tocotrienol | 29.93 |
| ethyl arachidonate | 28.72 |
| Aluminum phenol sulfonate | 27.39 |
| BENZALKONIUM BROMIDE | 26.00 |
| .alpha.-linolenic acid methyl ester | 24.93 |
| Orange Flower Extract OS | 22.78 |
| palmitamide mea | 13.20 |
| Octoxynol-9 | 9.83 |

The data in TABLE 11 showed the rank order of Lipopolysaccharide inhibition as measured by the LAL method. Molecules with the lipopolysaccharide inhibition of >50% would provide enough lipopolysaccharide inhibition to have an effect on reducing the lipopolysaccharide in vivo treatment applications, such as when delivered from a dentifrice or toothpaste. However, some of the tested molecules may interact with the enzymes in the LAL method to give false positive readings, the BODIPY method was used to further confirm the activity of the detoxifying agents in binding to virulence factors, such as LPS and LTA.

TABLE 12
LPS Inhibition Via BODIPY Method

| | % LPS |
|---|---|
| STRUCTURE_NAME | Displacment |
| palmitamide mea | 295.10 |
| Myristyl-.gamma.-picolinium chloride | 262.64 |
| potassium linoleate | 153.94 |
| Sodium hexadecyl sulfate | 124.89 |
| Aluminum phenol sulfonate | 109.40 |
| fenugreek | 107.75 |
| Steapyrium chloride | 102.04 |
| .alpha.-glyceryl monooloeate | 92.32 |
| Tin tetrachloride | 89.26 |
| Ferric chloride hexahydrate | 82.16 |
| STEARYLDIETHANOLAMINE | 78.64 |
| Dimethylaminopropyllaurylamide | 74.49 |
| Stearylamidopropyldimethylamine | 72.47 |
| Lavender Eucalyptus blend | 70.84 |
| Laureth-9 | 70.45 |
| linoleamide | 66.62 |
| Lavender Oil | 64.00 |
| diglycol monooleate | 63.16 |
| Diethylaminoethyl stearamide | 60.23 |
| Laureth-6 | 59.27 |
| N-Laurylpyridinium chloride | 57.72 |
| N-Myristoylglycine | 56.24 |
| N-Lauryldiethanolamine | 55.07 |
| MANGANESE ACETATE | 52.70 |
| Laurylisoquinolinium bromide | 52.58 |
| Oxirane, polymer with formaldehyde and | 51.90 |
| 4-(1,1,3,3-tetramethylbutyl)phenol | |
| ethyl arachidonate | 48.38 |
| Methylbenzethonium chloride | 47.52 |
| Dimethyldodecylbenzylammonium | 47.47 |
| chloride | |
| Arisaema Amurense Rhizome | 45.85 |
| BENZALKONIUM BROMIDE | 45.31 |
| Dioctyldimethylammonium chloride | 44.16 |
| Domiphen bromide | 43.50 |
| Octoxynol-9 | 42.56 |
| Myristyl-.gamma.-picolinium chloride | 41.80 |
| Steartrimonium chloride | 40.81 |
| ethyl cis-9-hexadecenoate | 39.25 |
| Quaternium-17 | 38.09 |
| Dimethyldodecylbenzylammonium | 37.86 |
| bromide | |
| DODECYLTRIPHENYLPHOSPHONIUM | 37.20 |
| BROMIDE, 98% | |
| Abacross Pepper Black Oil | 35.88 |
| Tetradecylbenzyldimethylammonium | 35.48 |
| chloride | |
| (z,z,z)-9,12,15-octadecatrienoic acid ethyl | 34.15 |
| ester | |
| Cetrimonium chloride | 34.00 |
| Didecyldimethylammounium chloride | 32.86 |
| Sodium pentadecyl sulfate | 32.82 |
| Oleyl amine | 30.12 |
| Dimethylbenzylcetylammonium chloride | 29.94 |
| Cetyltrimethylammonium bromide | 29.07 |
| Hexaethylene glycol, monodecyl ether | 28.94 |
| .alpha.-linolenic acid methyl ester | 27.98 |
| Benzalkonium chloride | 25.75 |
| Stearalkonium chloride | 23.93 |
| Cetylpyridinium chloride | 23.42 |
| Benzethonium chloride | 22.75 |
| .alpha.-linoleic acid | 22.30 |
| N-Lauryldiethanolamine | 20.21 |
| 9-Aminoacridine hydrochloride | 19.18 |
| monohydrate | |
| TRIDECYL SULFONIC ACID SODIUM | 18.48 |
| SALT | |
| methyl linoleate | 17.85 |
| Laurylisoquinolinium bromide | 16.86 |
| .beta.-Cyclodextrin,2-hydroxypropyl | 16.01 |
| ethers | |

TABLE 12 showed molecules which inhibited LPS by using the BODIPY method. Some molecules, such as palmitamide monoethanolamine, potassium linoleate, and myristyl gamma picolinium chloride caused a greater than 100% displacement. This is likely due to them enhancing the fluorescence of the BODIPY dye. The confirmatory test is to show displacement using both BODIPY and LAL, which is shown below in TABLE 13.

TABLE 13
LPS Inhibition Via BODIPY and LAL Cross Comparision

| | LAL % | BODIPY % LPS |
|---|---|---|
| STRUCTURE_NAME | Inhibition | Displacment |
| Myristyl-.gamma.-picolinium | 95.96 | 262.64 |
| chloride | | |
| potassium linoleate | 98.92 | 153.94 |
| Sodium hexadecyl sulfate | 89.44 | 124.89 |
| fenugreek | 90.00 | 107.75 |
| Steapyrium chloride | 100.13 | 102.04 |
| .alpha.-glyceryl monooloeate | 48.98 | 92.32 |
| Tin tetrachloride | 86.74 | 89.26 |
| Ferric chloride hexahydrate | 58.13 | 82.16 |
| STEARYLDIETHANOLAMINE | 88.09 | 78.64 |
| Dimethylaminopropyllaurylamide | 99.40 | 74.49 |
| Stearylamidopropyldimethylamine | 83.77 | 72.47 |
| Lavender Eucalyptus blend | 44.25 | 70.84 |
| Laureth-9 | 99.92 | 70.45 |
| linoleamide | 75.25 | 66.62 |
| Lavender Oil | 58.69 | 64.00 |
| diglycol monooleate | 67.01 | 63.16 |
| Diethylaminoethyl stearamide | 97.42 | 60.23 |
| Laureth-6 | 100.44 | 59.27 |
| N-Laurylpyridinium chloride | 96.53 | 57.72 |
| N-Myristoylglycine | 91.40 | 56.24 |
| N-Lauryldiethanolamine | 100.05 | 55.07 |
| Laurylisoquinolinium bromide | 97.41 | 52.58 |
| Methylbenzethonium chloride | 92.98 | 47.52 |
| Dimethyldodecylbenzylammonium | 98.25 | 47.47 |
| chloride | | |
| Arisaema Amurense Rhizome | 56.66 | 45.85 |
| Dioctyldimethylammonium | 93.30 | 44.16 |
| chloride | | |
| Domiphen bromide | 95.54 | 43.50 |
| Myristyl-.gamma.-picolinium | 96.23 | 41.80 |
| chloride | | |
| Steartrimonium chloride | 71.27 | 40.81 |

The compounds shown in TABLE 13 have inhibition using the LAL method and the BODIPY method. These molecules would be ideal candidates for inhibiting LPS in a biological system, such as in a gingival pocket to prevent or reduce gingivitis or periodontitis.

### Example 29—Stannous Fluoride Binds to Bacterial Cell Wall Components

Bacteria are coated with a well-developed cell wall which is responsible for many of their unique biological structures. The primary function of the cell wall is to protect the cell from internal turgor pressure caused by the much higher concentrations of proteins and other molecules inside the cell compared to its external environment. The bacterial cell wall is composed of peptidoglycan which is located immediately outside of the cytoplasmic membrane, lipoteichoic acids (LTA), lipopolysaccharides (LPS) and others.

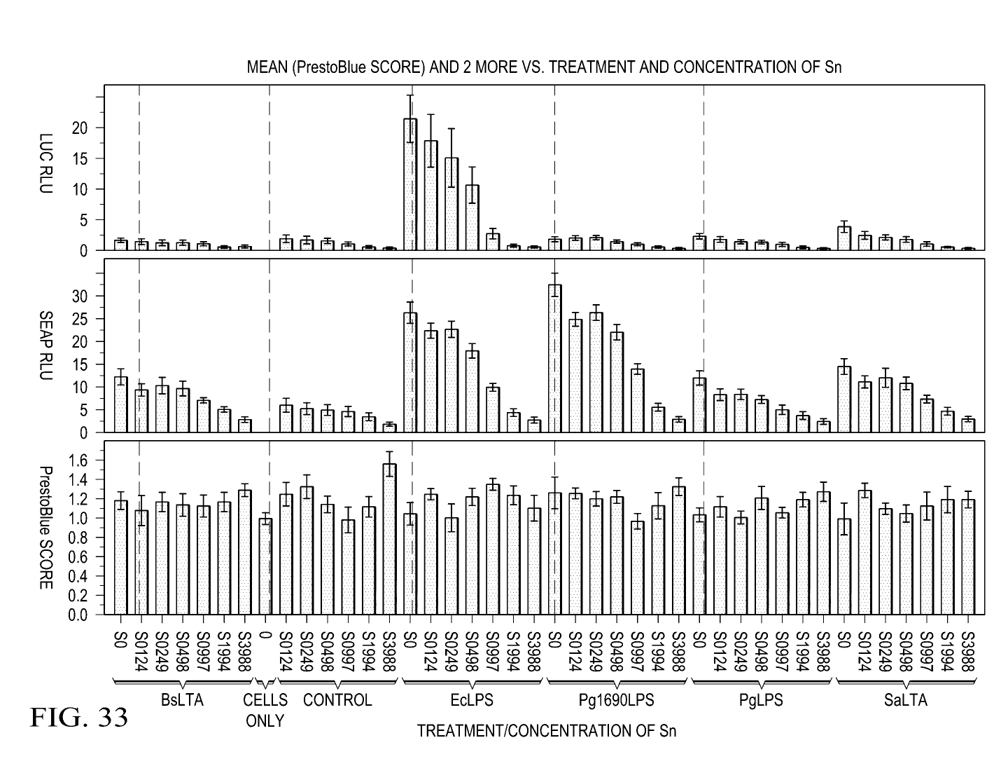

Stannous fluoride (SnF₂) was shown to bind to E. coli LPS as shown in FIG. 33. Experiments were carried out, in a procedure as described in EXAMPLE 11, to determine whether SnF₂ inhibited expression of the NFkB-SEAP and IRF-Luciferase reporter genes in THP-1 dual cells that were stimulated by LPS and LTA from different bacteria. In the experiments, various concentrations of SnF₂ were applied to the THP-1 dual reporter cells. As shown in FIG. 33, SnF₂ inhibited expression of the NFKB-SEAP reporter gene by LPS and LTA in a dose dependent manner. Only E. coli LPS promoted strong expression of the IRF-luciferase reporter gene in the THP-1 dual cells. As shown in FIG. 33, SnF₂ inhibited luciferase production in the THP-1 dual cells stimulated by E. coli LPS.

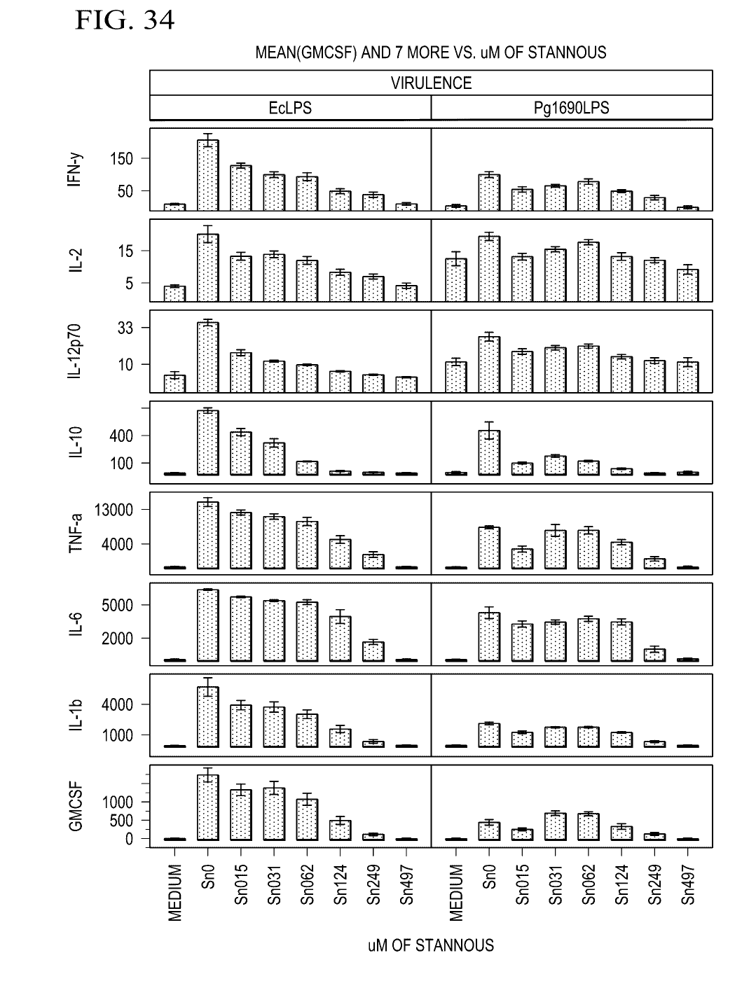

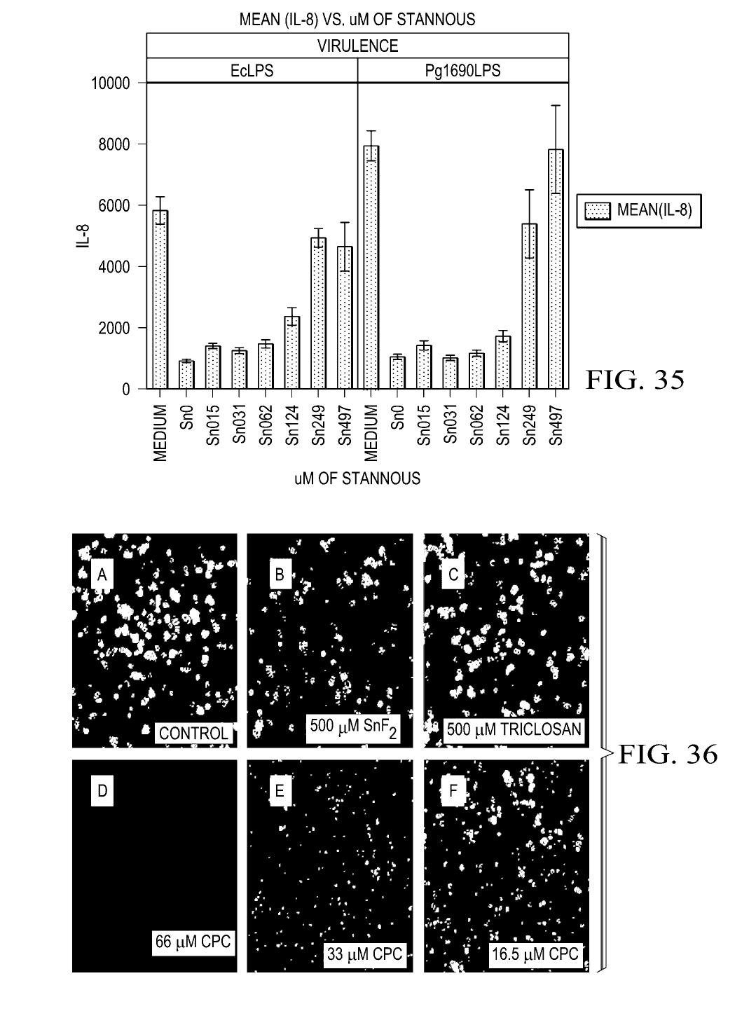

To determine whether SnF₂ can inhibit LPS-stimulated production of proinflammatory cytokines in human primary monocytes, various concentrations of SnF2 were applied to monocyte culture in the presence of 100 ng of E. coli LPS or P. gingivalis LPS 1690 for 24 hours. The primary human monocytes were purchased from Astarte Biologics (Bothell, Wash.). The cells (200,000 cells) were cultured in 200 μl of DMEM+glutaGRO supplemented with 9.1% fetal bovine serum and 1% penicillin/streptomycin, and treated with LPS and various concentrations of SnF₂. The medium was harvested at 24 hours after adding LPS and SnF2, and analyzed for proinflammatory cytokines in a kit following manufacturer's instruction (Meso Scale Diagnostics, Rockville, Md.). As shown in FIG. 34, SnF₂ inhibited cytokine production, stimulated by LPS, in a dose-dependent manner. Surprisingly, neither E. coli LPS nor P. gingivalis LPS 1690 stimulated production of IL-8 in primary human monocytes at 100 ng/ml, as shown in FIG. 35. Instead, they suppressed production of IL-8. SnF₂ reversed the suppression of LPS on IL-8 production in a dose-dependent manner, as shown in FIG. 35.

### Example 30—Stannous Fluoride Blocks Fluorescence-Labeled E. coli LPS from Binding to TLR4 Receptors

Procedures: Green-fluorescent Alexa Fluor 488 conjugated LPS was purchased from Thermo Fisher, Waltham, Mass. HEK-Blue-hTLR4 cell line was purchased from InvivoGen as described in EXAMPLE 5. Stannous fluoride, Cetylpyridium chloride (CPC) and Triclosan were purchased from Sigma-Aldrich, St. Louis, Mo. The cells were grown and maintained in the 75 cm² flasks with 15 ml growth medium at 37° C. incubator, 95% humidity and 5% CO₂. The growth medium: DMEM, with 4.5 g/l glucose, supplemented 10% fetal bovine serum, 50 U/ml penicillin and streptomycin, 100 μg/ml Normocin, and 2 mM L-glutamine, purchased from Thermo Fisher, Waltham, Mass.

### LPS-TLR4 Binding:

A. Make solution A (3× concentration of the test compound solution in the cell growth medium).
B. Make solution B (3×(3 ug/ml) LPS+0.15 ug/ml LPS binding protein in the cell growth medium).
C. Take one flask of HEK-blue-TRL4, the cells were detached directly from in growth medium by hand shaking, the suspend cells were transferred into a 50 μl tube, after 3 minutes centrifugation at low speed (800 rpm), the cells were re-suspended in 1.5-2 ml of the cell growth medium.
D. Add 100 μl of solution B, add 100 μl of solution A (the test compounds: SNF2, CPC and triclosan), add same amount of culture medium, add 100 μl of HEK-blue-TRL4 into a microfuge tube.
E. Place the microfuge tube inside an incubator at 35° C. with gently shaking for 1 hours
F. Centrifuge the microfuge tube at low speed (<1000 rpm) 1-2 min to precipitate cells
G. Remove supernatant and add 1 ml of the assay buffer (1×HBSS, 20 mM HEPES, pH 7.4) to re-suspend the cell.
H. Repeat procedures F and H once.
I. Re-suspend the cells in 310 μl of the assay buffer
J. Aliquot 100 μl of cells from the microfuge tube in procedure I into 96-well assay plate (Black wall and clear bottom).
K. Perform imaging and analysis using ImageXpress and MetaXpress software (Molecular Devices, Sunnyvale, Calif.).

Results: Strong green fluorescence was observed on the surface of HEK-Blue-hTLR4 in the control samples (without test compounds) as shown in FIG. 36, indicating labeled LPS binding to the TLR4 receptors located on the cell surface. Stannous fluoride and CPC reduced fluorescence intensity significantly (TABLE 14).

TABLE 14
Inhibition of E. coli LPS binding to
HEK-blue-TLR4 cells by SnF₂ and CPC.

| | Average fluorescence | STDEV | % of |
|---|---|---|---|
| | intensity (FI) | (FI) | control |
| Control | 1027 | 68.5 | 100 |
| 500 μM SnF2 | 662 | 54.6 | 64.5 |
| 66 μM CPC | 0 | 17.4 | 0 |
| 33 μM CPC | 368 | 25.4 | 35.8 |
| 16.5 μM CPC | 635 | 48.4 | 61.8 |
| 500 μM triclosan | 1021 | 56.7 | 99.4 |

### Example 31—Lipopolysaccharides (LPS) and Bacteria Stimulated Production of Proinflammatory Cytokines in Human Peripheral Blood Mononuclear Cells (PBMC)

Human peripheral blood mononuclear cells (PBMC) were isolated from blood obtained from Gulf Coast Regional Blood Center, Houston, Tex., USA, using Histopaque 1077 (Sigma Aldrich Co., St. Louis, Mo., USA) and Leucosep tubes (Greiner Bio-One, Monroe, N. Carolina, USA). The cells were cultured in 200 μl of RPMI 1640 medium in each well of a 90-well plate (ThermoFisher Scientific, Inc., Grand Island, N.Y., USA) containing 10% fetal bovine serum and 1% penicillin/streptomycin antibiotics at 37° C. with a 5% CO2 atmosphere. P. gingivalis LPS and E. coli LPS were purchased from Invivogen (San Diego, Calif.). P. gingivalis, P. pallens, and F. alocis were grown as described in EXAMPLE 1.

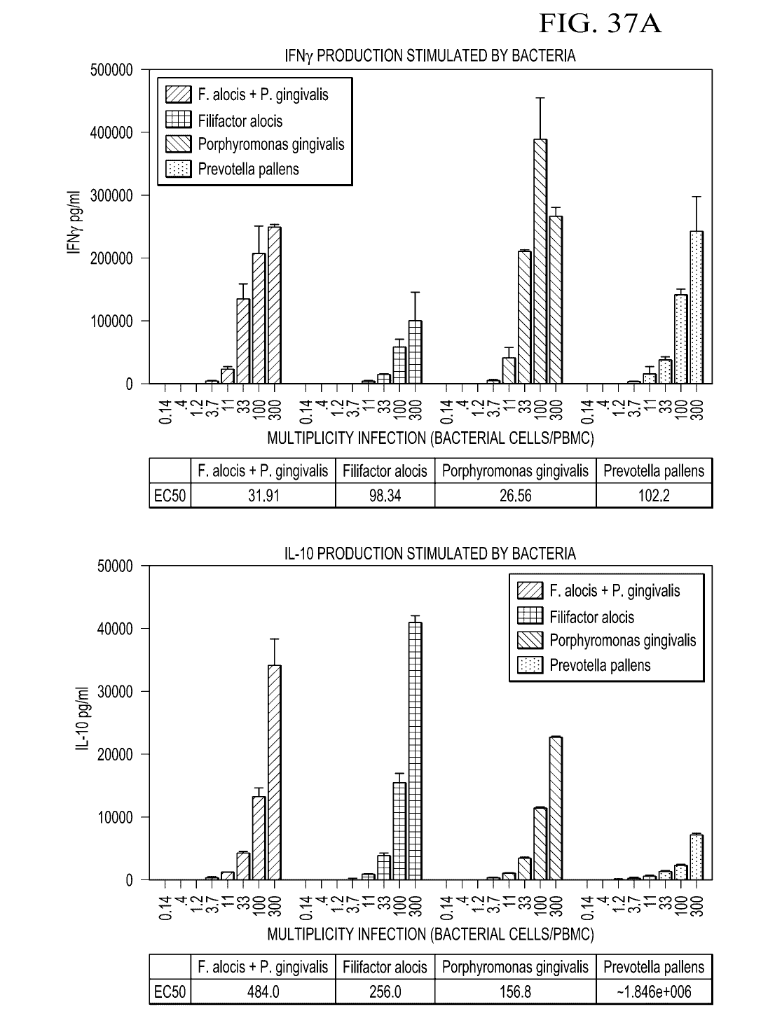

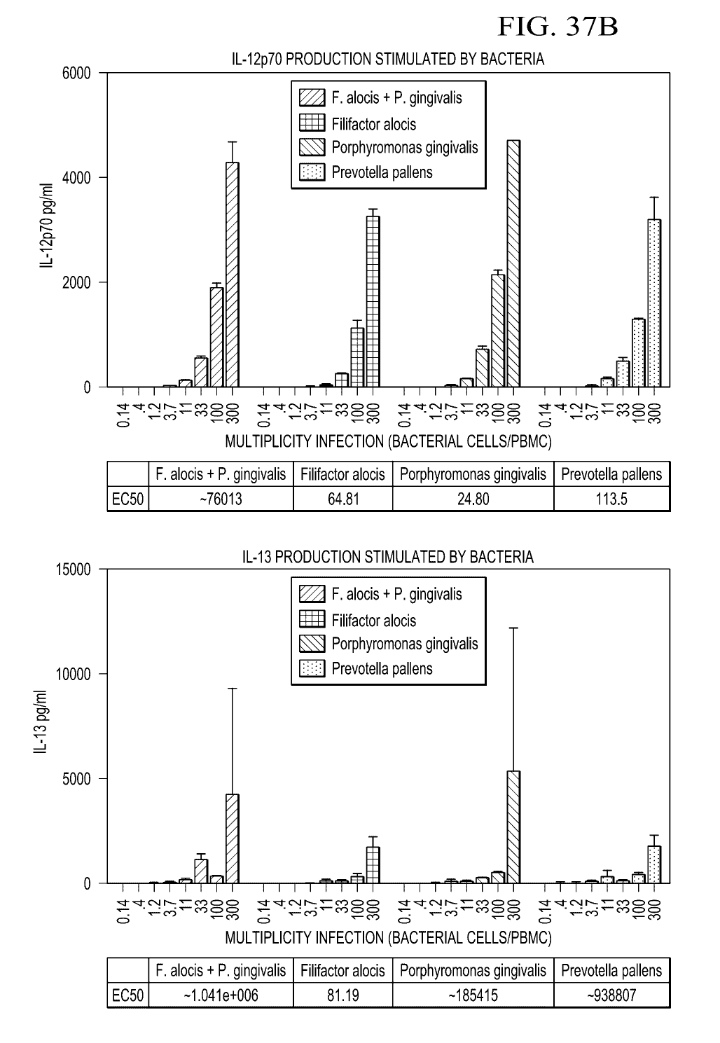

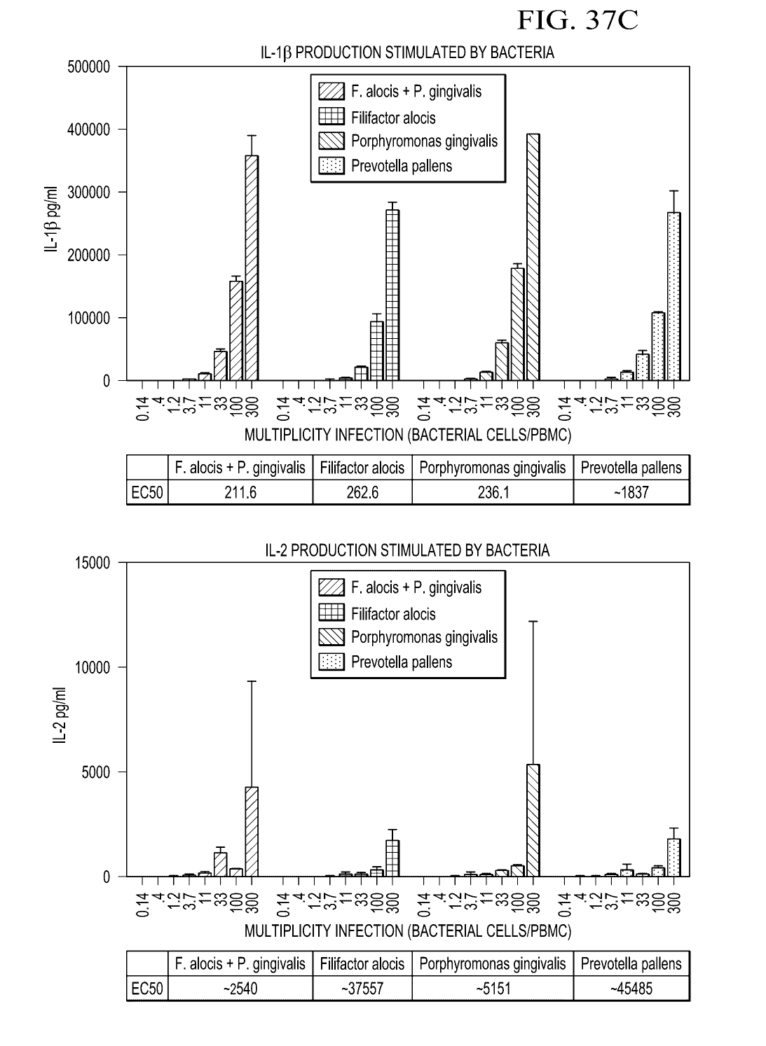

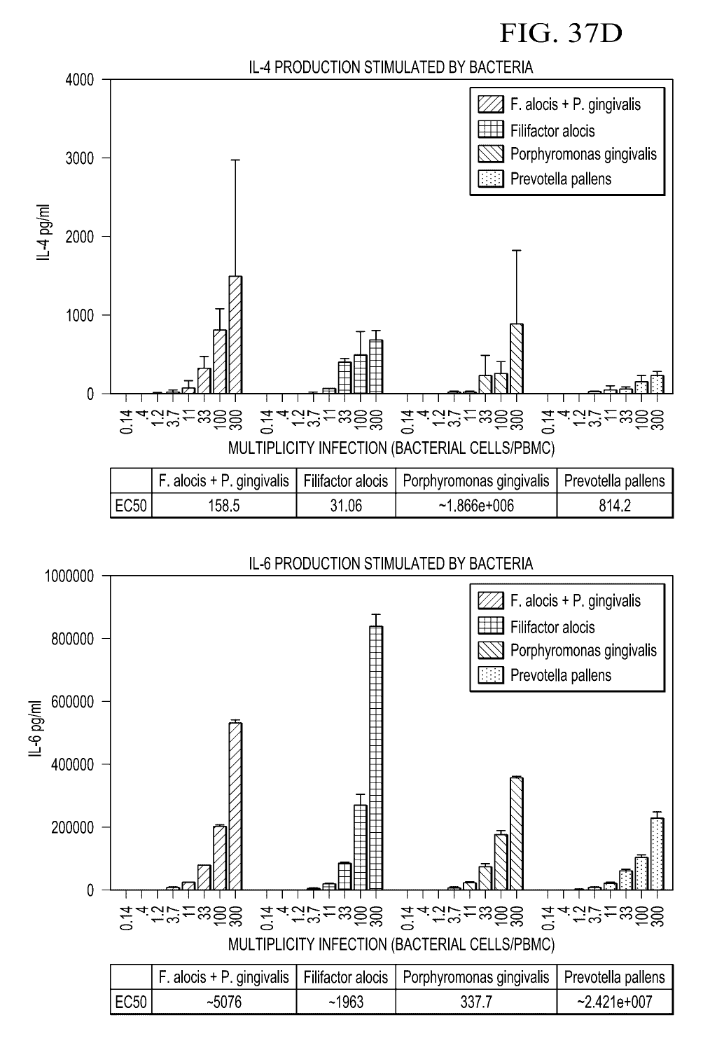

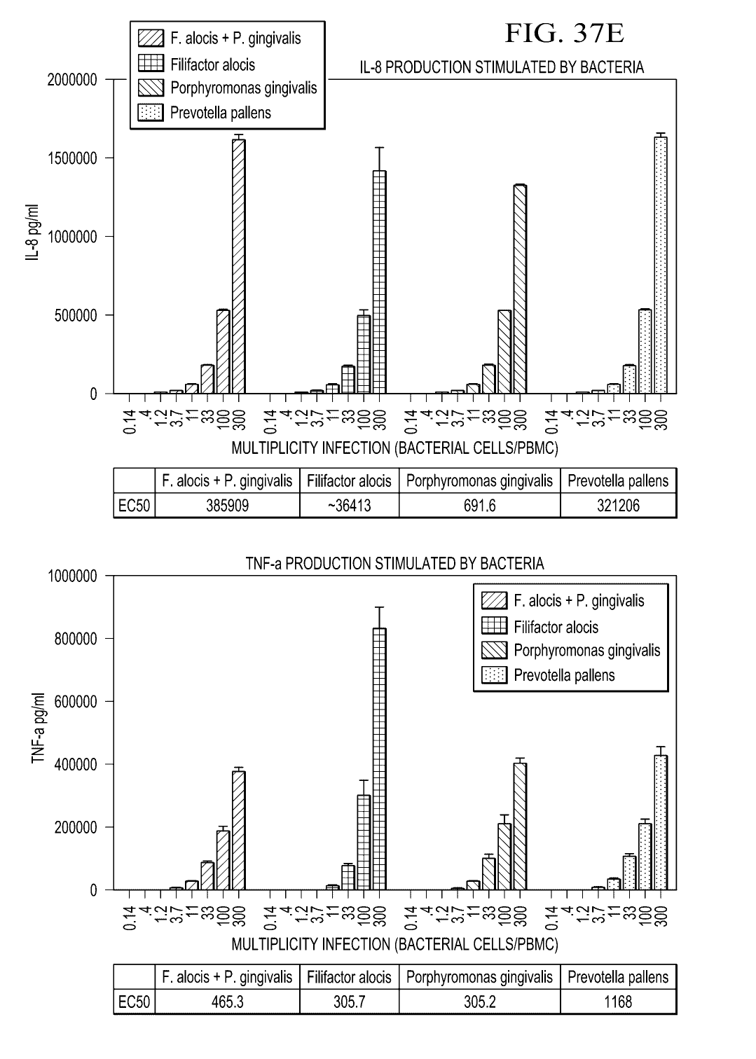

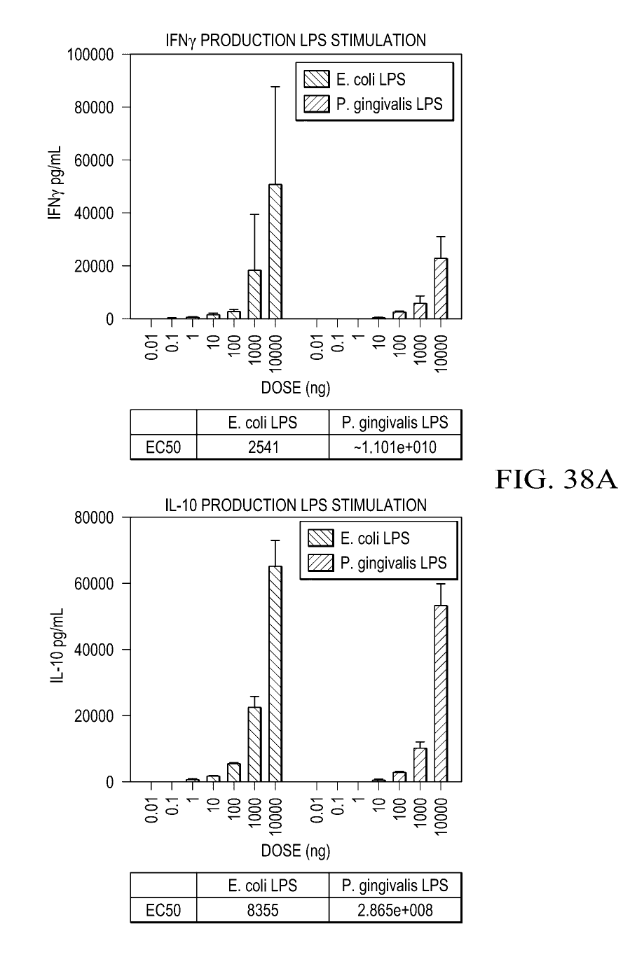

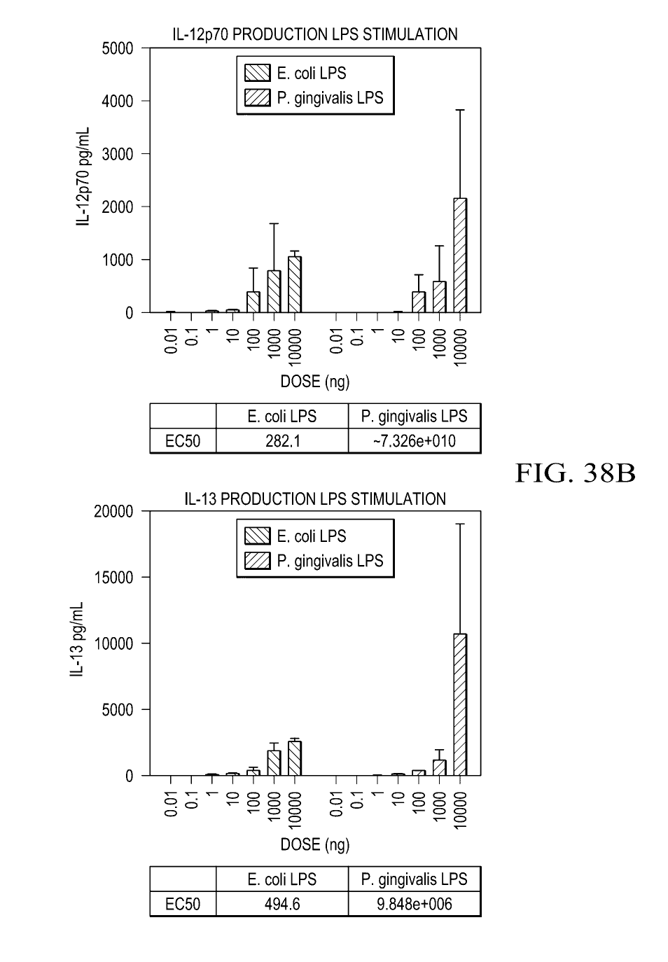

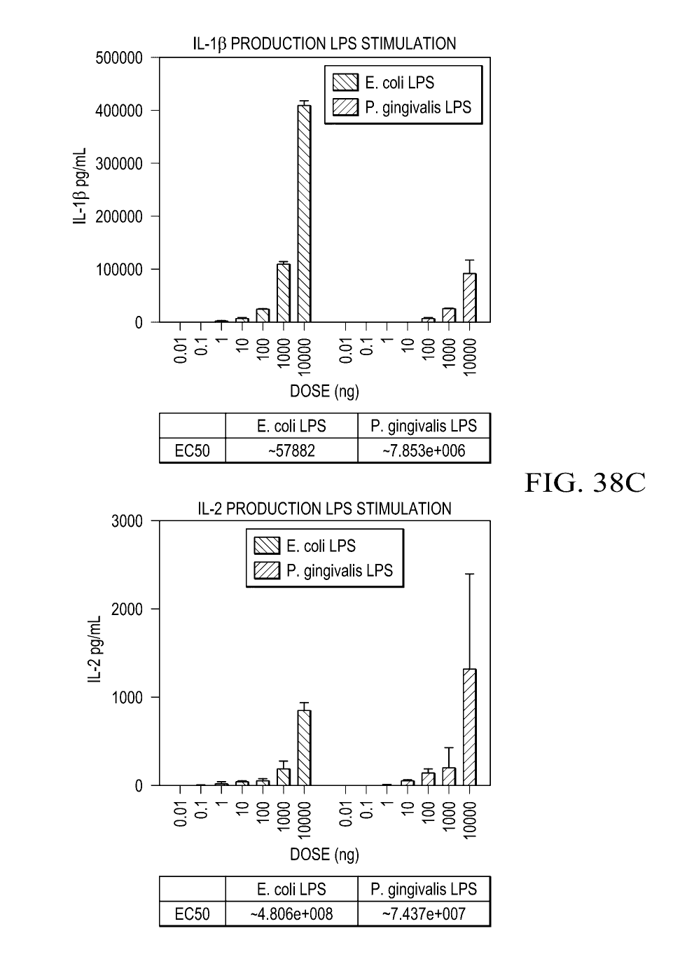

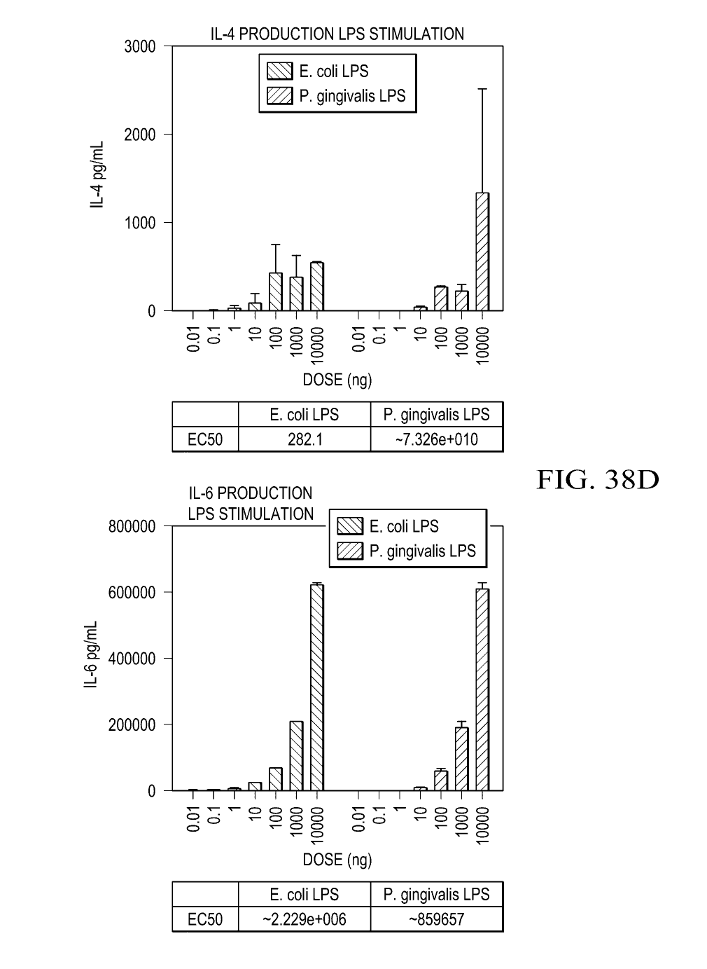

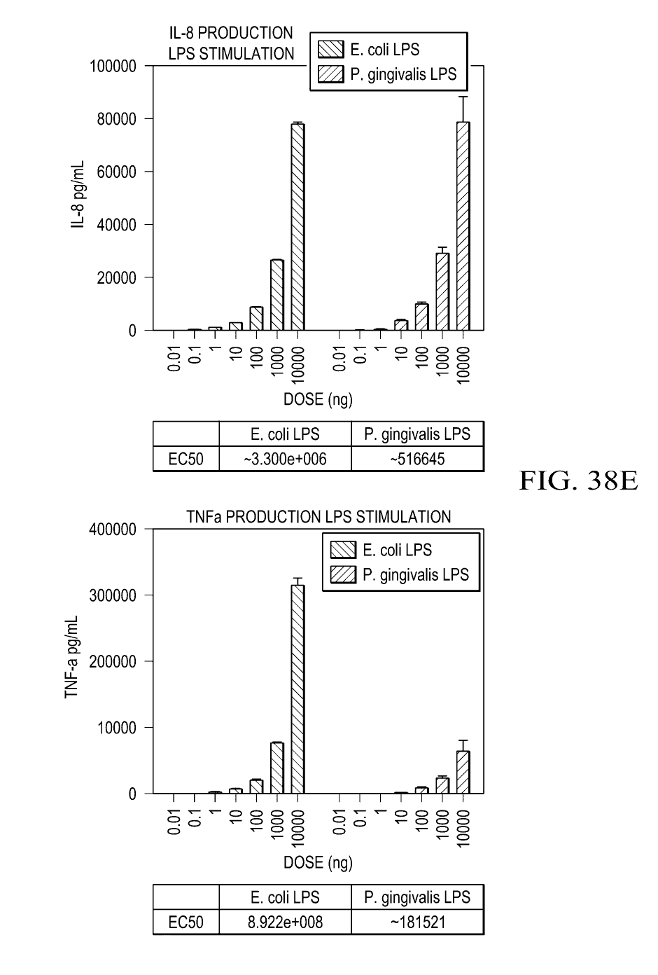

The human PBMC cells were seeded onto 96-well at 200,000 cells per well in 200 μl of RPMI 1640 medium in each well (ThermoFisher Scientific, Inc., Grand Island, N.Y., USA) containing 10% fetal bovine serum and 1% penicillin/streptomycin antibiotics, and treated with clinical bacteria and LPS. The cells were then incubated for 24 hours at 37° C. with a 5% CO₂ atmosphere. The conditioned media were harvested at the end of experiment and analyzed for proinflammatory cytokines in an ELISA kit following manufacturer's instruction (Meso Scale Diagnostics, Rockville, Md.). As shown in FIGS. 37A, B, C, D and E, all bacteria stimulated production of proinflammatory cytokines. Similarly, both E. coli LPS and P. gingivalis LPS 1690 also promoted production of proinflammatory cytokines in human PBMC (FIGS. 38A, B, C, D and E).

### Example 32—Application of Lipopolysaccharide Mitigation Chemistry to an Oral Tooth Strip

### Methods of Use.

Methods of using the desensitizing delivery system set forth herein to treat the oral cavity (or portions thereof) before or after the use of a whitening delivery system (such as a commercially available whitening strip product like CREST WHITESTRIPS) are also contemplated herein.

Examples of lipopolysaccharide inactivation compositions applied to a tooth strip according to the present invention are found below in Tables 15A, 15B, 15C, and 15D. The compositions were compounded using either a Ross double planetary mixer LDM-2 or Ross double planetary mixer DPM-40. Glycerin was weighed and added to a clean Ross mix tank. Water was weighed and added to a separate stainless steel mix tank equipped with a lightning mixer and 4″ diameter turbine mixing blade. The carboxymethyl cellulose and the CARBOPOL were weighed and added to a clean plastic polyethylene bucket. The lid was applied to the bucket and the powders were rotated by hand to blend the powders together for 10 minutes. The potassium sorbate, potassium benzoate and potassium oxalate were weighed and added to the water in the stainless steel mixing tank. The lightning mixer was then used to completely dissolve the added salts. The sodium hydroxide was weighed and added to the potassium sorbate/sodium benzoate/potassium oxalate solution in the stainless steel mix tank. The solution was then mixed until clear. The blended carboxymethyl cellulose and CARBOPOL powder mix was carefully added to the glycerin by hand adding to cover the surface of the glycerin uniformly in the Ross mix tank. The Ross mixer was started and ran at a speed setting of 5 for 30 minutes. The solution from the stainless steel tank was then added to the Ross mixer and mixing continued for 45 minutes at a speed setting of 5. After this mixing session, the composition was checked for visible lumps. If lumps were present, the composition was mixed for an additional 20 minutes at a speed setting of 5.

Once the compounding of the composition was complete, strip-type delivery systems were made by slot coating the composition onto a sheet of release liner made of fluoropolymer coated polypropylene film sold under the brand name SCOTCHPACK 9741 RELEASE LINER (commercially available from 3M (Minnesota, USA)) and then combined with a polyethylene film material, an HDPE/LDPE blend polyethylene film (commercially available from Clopay (Cincinnati, USA) as embossed polyethylene film—32 GSM Sof-flex) using a continuous lamination process. This resulting laminate was then run through rotary kiss cutting dies to cut the strip shape to 1 cm×3 cm. The polyethylene material outside of the strip perimeter was removed and the resultant release liner with 1 cm×3 cm strip spaced out on the web was cut into individual release liners measuring 3 cm×9 cm. The individual release liners with strips were placed into foil laminate pouches and sealed with heat.

The resulting delivery systems were tested for release off the release liner and gel flow and the Complex Modulus and Delta were measured and calculated pursuant to the methods set forth herein. Some products were further tested for in vivo adhesion and the results included below. To determine release off the liner, the strip was removed from the foil laminate pouch and the strip and laminated composition were grasped between two fingers and pulled from the release liner at a rate of approximately 3 cm/sec. The percentage amount of composition remaining on the release liner was visually analyzed and recorded. In vivo adhesion was determined by a wear panel.

The delivery systems with the compositions shown in Table 15 are according to the invention set forth herein. As may be seen by the data tabulated in the Table below, compositions 15a through 15E, were found to perform acceptably in all categories tested.

TABLE 15
Desensitizing Oral Care Compositions

| | 15A | 15B | 15C | 15D | 15E |
|---|---|---|---|---|---|
| Ingredient | Wt. % | Wt. % | Wt. % | Wt. % | Wt. % |
| Carboxymethyl | 5.0 | 5.0 | 4.5 | 4.5 | 4.0 |
| Cellulose 7M8SF | | | | | |
| Glycerin USP | 31.86 | 31.86 | 31.86 | 31.86 | 31.86 |
| (99.7) | | | | | |
| CARBOPOL 956 | 1.0 | 1.0 | 2.5 | 2.5 | 2.5 |
| Sodium Benzoate, | 0.50 | 0.5 | 0.5 | 0.5 | 0.5 |
| NF FCC | | | | | |
| Potassium Sorbate | 0.20 | 0.2 | 0.2 | 0.2 | 0.2 |
| Sodium Hydroxide | 0.25 | 0.25 | 1.1 | 1.05 | 1.0 |
| Solution 50 | | | | | |
| Myristyl-.gamma.- | 0.1 | | | | |
| picolinium chloride | | | | | |
| potassium linoleate | | 0.3 | | | |
| Hexaethylene | | | 0.4 | | |
| glycol, monodecyl | | | | | |
| ether | | | | | |
| Palmitamide | | | | 0.4 | |
| ethanolamine | | | | | |
| Stannous fluoride | | | | | 0.3 |
| Deionized water | QS | QS | QS | QS | QS |

Carboxymethyl Cellulose 7M8SF, 9H, and 7H are part of the AQUALON series commercially available from Hercules.
CARBOPOL 956 is commercially available from Lubrizol.
*Data point not collected for sample.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for identifying agents for detoxifying oral virulence factors comprising:
a) providing a reporter cell that expresses at least one of TLR2 or TLR4;
b) providing an oral virulence factor;
c) providing a potential detoxifying agent;
d) combining oral virulent factor and potential detoxifying agent with reporter cell;
e) determining EC50 of virulence factor.

2. The method of claim 1, wherein the virulence factor is lipopolysaccharide.

3. The method of claim 2, wherein the lipopolysaccharide is from a Gram negative bacterium.

4. The method of claim 1, wherein the virulence factor is lipoteichoic acid.

5. The method of claim 4, wherein the lipoteichoic acid is from a Gram positive bacterium.

6. The method of claim 1, wherein the potential detoxifying agent is at least one of myristyl-gamma-picolinium chloride; potassium linoleate; Sodium hexadecyl sulfate; fenugreek; steapyrium chloride; alpha.-glyceryl monooloeate; stearyldiethanolamine; Dimethylaminopropyllaurylamide; Stearylamidopropyldimethylamine; linoleamide; N-Lauryldiethanolamine; Steartrimonium chloride; hexaethylene glycol, monodecyl ether; Laureth-6; Laureth-9; Quaternium-17; Bromoenol lactone; Oleyl amine; cetrimonium chloride; or palmitamide monoethanolamine.

7. A method to identify detoxifying agents that bind to at least one of lipopolysaccharide or lipoteichoic acid comprising:
a) providing a reporter cell that expresses at least one of TLR2 or TLR4;
b) providing at least one of lipopolysaccharide or lipoteichoic acid;
c) combining at least one of lipopolysaccharide or lipoteichoic acid and potential detoxifying agent with reporter cell;
d) measuring binding of potential detoxifying agents using at least one of BODIPY method or LAL method of low EC50 lipopolysaccharide or lipoteichoic acid; and
e) determining EC50 of at least one of lipopolysaccharide or lipoteichoic acid.

8. An oral care composition comprising:
a detoxifying agent that is at least one of myristyl-gamma-picolinium chloride; potassium linoleate; Sodium hexadecyl sulfate; fenugreek; steapyrium chloride; alpha.-glyceryl monooloeate; stearyldiethanolamine; Dimethylaminopropyllaurylamide; Stearylamidopropyldimethylamine; linoleamide; N-Lauryldiethanolamine; Steartrimonium chloride; hexaethylene glycol, monodecyl ether; Laureth-6; Laureth-9; Quaternium-17; Bromoenol lactone; Oleyl amine; cetrimonium chloride; or palmitamide monoethanolamine.

9. The oral care composition of claim 8 wherein the detoxifying agent is present in an amount of from about 0.001% to about 2.0%, by weight of the oral care composition.

10. The oral care composition of claim 8, wherein the oral care composition is a strip.


SEQUENCE LISTING (9 sequences)

SEQ ID NO: 1 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 2 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 3 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 4 - protein, 798 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATS

SEQ ID NO: 5 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 6 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGPTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 7 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 8 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 9 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE